(19) United States
(12) Patent Application Publication
(10) Pub. No.: US 2023/0233674 A1
(43) Pub. Date: Jul. 27, 2023

(54) INTRANASAL VACCINES AND THERAPEUTICS FOR RESPIRATORY DISEASES

(71) Applicant: UNIVERSITY OF HOUSTON SYSTEM, Houston, TX (US)

(72) Inventors: Navin Varadarajan, Houston, TX (US); Ankita Leekha, Houston, TX (US)

(73) Assignee: UNIVERSITY OF HOUSTON SYSTEM, Houston, TX (US)

(21) Appl. No.: 18/100,456

(22) Filed: Jan. 23, 2023

Related U.S. Application Data
(60) Provisional application No. 63/301,918, filed on Jan. 21, 2022.
(60) Provisional application No. 63/329,261, filed on Apr. 8, 2022.

(51) Int. Cl. A61K 39/39; A61P 31/14; A61P 31/16; A61K 39/215

(52) U.S. Cl. CPC A61K 39/39; A61P 31/14; A61P 31/16; A61K 39/215; A61K 2039/543

(57) ABSTRACT

Described herein are compositions and methods for the reducing a risk of contracting a respiratory disease in a subject or reducing a risk of transmitting a respiratory disease from a first subject to a second subject. In some cases, a composition or method described herein can comprise a modulator (e.g., a pattern recognition receptor, such as a STING agonist, for instance cGAMP) In some cases, a composition or method described herein can comprise a liposome, which may be used to encapsulate one or more STING agonists. In some cases, a liposome of a composition or method described herein may comprise one or more antigens attached to, integrated into, or associated with a liposomal membrane of the liposome.

### CROSS-REFERENCE TO RELATED APPLICATIONS

The present application claims the benefit of U.S. Provisional Patent Application No. 63/301,918, filed on Jan. 21, 2022 and U.S. Provisional Patent Application No. 63/329,261, filed on Apr. 8, 2022. The entirety of each of the aforementioned applications is incorporated herein by reference for all purposes.

### SEQUENCE LISTING

The instant application contains a Sequence Listing which has been submitted electronically in XML format and is hereby incorporated by reference in its entirety. Said XML copy, created on Jan. 23, 2023, is named AF23853.P179WO.xml and is 52,000 bytes in size. Applicant hereby incorporates by reference the material in the Sequence Listing.

### BACKGROUND

Safe and durable preventative and therapeutic compositions are urgently needed to address various diseases, such as infections caused by respiratory pathogens. Numerous embodiments of the present disclosure address the aforementioned need.

### BRIEF DESCRIPTION OF THE DRAWINGS

FIG. 1 provides a depiction of a composition comprising a liposome, a payload, and an antigen for treating or preventing a disease, in accordance with various embodiments of the present disclosure.

FIGS. 2A and 2B illustrate methods of treating or preventing a disease in a subject.

FIG. 3A shows an overall schematic of formulations and intranasal delivery of compositions (e.g., NanoSTING compositions) to animals according to embodiments described herein. FIG. 3B illustrates signaling pathways that can be involved in (e.g., activated by) embodiments of methods and compositions (e.g., comprising NanoSTING delivery). In some cases, THP1-Lucia™ cells can be used to stably express the secreted form of luciferase under the control of a synthetic interferon responsive promoter, for example, to illustrate Type I interferon (IFN) induction (e.g., via IRF pathway activation) by embodiments of compositions and methods described herein in an in vitro setting.

FIG. 3C and FIG. 3D illustrate distributions of NanoSTING liposomal particle sizes at 25 degrees Celsius and 37 degrees Celsius, respectively, as measured by dynamic light scattering, in accordance with embodiments.

FIG. 3E illustrates distributions of NanoSTING liposomal particle sizes as measured by dynamic light scattering, in accordance with embodiments. FIG. 3F illustrates zeta potential of NanoSTING compositions, in accordance with embodiments described herein, as measured by electrophoretic light scattering (ELS).

FIG. 3G shows a table of particle sizes (DH), polydispersity index (PDI), and zeta potential for compositions (e.g., NanoSTING particles) described herein at 25 degrees Celsius and 37 degrees Celsius.

FIG. 4 illustrates kinetics of the induction of luciferase in THP1-dual cells by varying concentrations of the NanoSTING, in accordance with embodiments. RLU: relative light units.

FIG. 5A illustrates experimental design for experiments in which 3-4 Balb/c mice were treated with single doses of NanoSTING (10 μg, 20 μg, or 40 μg). Blood, nasal turbinates, and lungs were analyzed at 6 h, 12 h, 24 h, 36 h, and 48 h from euthanized animals for cGAMP from nasal turbinate by ELISA (FIG. 5B), for cGAMP from lungs by ELISA (FIG. 5C), cGAMP from serum by ELISA (FIG. 5D), and for CXCL10, IFNB1, ISG15, IRF7, Mx1, Mx2, IL-6, TNF, IL-10, and Ifit1 by qRT-PCR (from RNA extracted from nasal turbinates) (FIGS. 5E, 5F, 5G, 5H, 5-I, 5J, 5K, 5L, 5M, and 5N, respectively, showing fold change in gene expression), IFNβ by ELISA, and CXCL10 by ELISA. The results illustrate methods of treating or preventing a disease in a subject. Analysis was performed using a Mann-Whitney test. Bars and columns show median values. Each dot represents individual mouse. Mann-Whitney test: ****p<0.0001; ***p<0.001; **p<0.01; *p<0.05; ns, not significant.

FIG. 6 shows DNA sequences of primers used to obtain qRT-PCR experiment data shown in FIGS. 5E-5N.

FIGS. 7A, 7B and 7C illustrate data from experiments described in FIG. 5A for IFNβ from nasal turbinate by quantitative ELISA (FIG. 7A), for CXCL10 from nasal turbinates (FIG. 7B), and lungs (FIG. 7C) by quantitative ELISA in accordance with embodiments. FIG. 7D shows quantification of cGAMP in mouse serum after treatment with NanoSTING, in accordance with embodiments. FIG. 7E shows detection of IFNβ concentration in mouse serum using quantitative ELISA, in accordance with embodiments. FIG. 7F detection of CXCL10 levels in mouse serum using quantitative ELISA, in accordance with embodiments. For fold changes in gene expression, analysis was performed using a Mann-Whitney test. Bars and columns show median values. Mann-Whitney test: ****p<0.0001; ***p<0.001; **p<0.01; *p<0.05; ns, not significant.

FIGS. 8A and 8B show body temperature change and body weight change, respectively, in studies of NanoSTING versus control (PBS) on hamster subjects, wherein groups of animals were administered with daily doses of 60 μg of NanoSTING intranasally (n=4/group) or PBS (n=4/group) for four consecutive days. Hamsters were euthanized on Day 5 after administering the last dose on Day 4, followed by the collection of lungs. FIG. 8C shows representative images of hamster nasal turbinates, lungs, and stomach dissected analyzed in Evan's blue dye assays. Hamsters (n=4/group) were intranasally administered with 0.125% Evans blue dye in PBS (40 μL and 120 μL). FIGS. 8D and 8E show quantified distribution of Evan's blue dye after intranasal administration. In these experiments, the supernatants from homogenized lungs and stomach were treated with trichloroacetic acid and analyzed absorbance at 620 nm. Concentrations of dye were interpolated from a standard curve.

FIG. 9A shows real-time qRT-PCR for fold induction of Cxcl11, Ccl5, Ifnb1, Isg15, Irf7, Mx1, Mx2, Il6, and Il10 mRNA from 60 μg NanoSTING treated hamster lungs compared with control hamster lungs (n=4). FIG. 9B shows DNA sequences of primers used to collect data for FIG. 9A.

FIGS. 10A, 10B, and 10C show activation of IFN-dependent and IFN-independent pathways in the lungs of hamsters treated with NanoSTING using RNA-sequencing. FIG. 10A shows a heatmap of top 50 differentially expressed genes (DEGs) between NanoSTING treated lungs (marked as green) and control lungs (marked as black). FIG. 10B shows volcano plots of DEGs comparing NanoSTING treated and PBS treated control animals. FIG. 10C shows geneset enrichment analyses (GSEA) of C2 and C7 curated pathways visualized using Cytoscape. Nodes (red and blue circles) represent pathways, and the edges (green lines) represent overlapping genes among pathways. The size of nodes represents the number of genes enriched within the pathway, and the thickness of edges represents the number of overlapping genes. The color of nodes was adjusted to an FDR q value ranging from 0 to 0.25. Clusters of pathways are labeled as groups with a similar theme.

FIG. 11A shows GSEA of IFN-independent activities of STING pathway activated in the lung of NanoSTING treated animals. The schematic represents the comparison that was made between samples collected from GSE149744 dataset to generate the pathway gene set. FIG. 11B shows expression of genes in lungs associated with IFN-dependent and IFN-independent antiviral pathways between NanoSTING and control groups. FIGS. 11C and 11D show schematics representing rate constants and equations governing viral dynamics during (FIG. 11C) natural infection and (FIG. 11D) in the presence of NanoSTING treatment. FIG. 11E shows reduction in the viral area under the curve (AUC) at different NanoSTING efficacies (RIR) compared to natural infection. The treatment is initiated on day 0 with the assumption that the effects of NanoSTING treatment only last for 24 h. FIG. 11F shows a heatmap of viral AUC with varying NanoSTING efficacy and treatment initiation time. The red box represents the combination with close to 100% reduction in viral AUC. FIG. 11G shows peak natural response is independent of the initial viral load. FIG. 11H shows viral dynamics can be independent of initial viral titer upon treatment with NanoSTING. E0 is the initial number of infected cells upon viral infection, which is a surrogate for viral titer. FIGS. 11-I, 11J, and 11K show evolution of viral dynamics with different treatment initiation time and NanoSTING efficacies. FIG. 11L shows a heatmap of viral AUC with varying NanoSTING efficacy and treatment initiation time when NanoSTING effects last for 48 h after treatment initiation.

FIGS. 12A, 12B, 12C, 12D, 12E, and 12F show NanoSTING protects hamsters during the primary challenge and prevents reinfection with the pathogenic SARS-CoV-2 Delta (B.1.617.2) after treatment with NanoSTING. For viral titers, analysis was performed using a Mann-Whitney test. FIG. 12A shows an experimental design in which groups of 12 hamsters were each treated with a single dose of 120 μg NanoSTING and later challenged with 10⁴ TCID50 of SARS-CoV-2 Delta variant on day 0 by the intranasal route. Viral titers of lung and nasal tissues were determined for half of the hamsters (n=6) on day 6. The remaining six hamsters were rechallenged on day 28 and the bodyweight change was tracked until day 35. FIGS. 12B and 12C show percentage weight change as compared to the baseline at the indicated time intervals. FIG. 12F shows percent bodyweight change monitored after rechallenge starting from day 28 until day 35. FIGS. 12D and 12E show viral titers measured by plaque assay in nasal tissues and lungs post day 6 of infection. Weight data was compared via mixed-effects model for repeated measures analysis. Each dot is an individual hamster. Bars and columns show median values. Mann-Whitney test: ****p<0.0001; ***p<0.001; **p<0.01; *p<0.05; ns, not significant.

FIGS. 13A, 13B, and 13C show single-dose prophylactic administration of NanoSTING compositions, in accordance with embodiments, protects against challenge with SARS-CoV-2 delta variant (B.1.617.2). Hamsters were treated with NanoSTING (120 micrograms) at 24 hours (e.g., “−1 day”) or 72 hours (e.g., “−3 days”) before challenge with SARS-CoV-2 delta variant. The treatment reduced viral replication and prevented weight loss. FIGS. 13A, 13B, and 13C show viral titers quantified in the lung and nasal tissue by plaque assay post day 2 after the challenge. For viral titers, analysis was performed using a Mann-Whitney test. Weight data was compared via mixed-effects model for repeated measures analysis. Each dot is an individual hamster, and bars indicate median values. Mann-Whitney test: ****p<0.0001; ***p<0.001; **p<0.01; *p<0.05; ns, not significant.

FIGS. 14A, 14B, and 14C show single-dose post-exposure administration of NanoSTING compositions, in accordance with embodiments, protects against challenge with SARS-CoV-2 delta variant (B.1.617.2). Groups of 6 hamsters were each challenged with SARS-CoV2 virus (Delta variant-B.1.617) and 6 h later treated with a single dose of NanoSTING (120 μg) (FIG. 14A). Animals were euthanized post-day 2 of infection and analyzed to determine viral loads in the lungs and nasal tissue. FIGS. 14B and 14C show viral titers from the lung and nasal tissue quantified by plaque assay post day 22 after the challenge. For viral titers, analysis was performed using a Mann-Whitney test. Weight data was compared via mixed-effects model for repeated measures analysis. Each dot is an individual hamster, and bars indicate median values. Mann-Whitney test: ****p<0.0001; ***p<0.001; **p<0.01; *p<0.05; ns, not significant.

FIGS. 15A, 15B, 15C, and 15D illustrate single-dose prophylactic administration of NanoSTING compositions, in accordance with embodiments disclosed herein, can protect against SARS-CoV-2 Delta variant. Hamsters were treated with NanoSTING (120 micrograms) 24 hours before challenge with SARS-CoV-2 Delta variant. The treatment reduced viral replication and prevented weight loss in treated animals.

FIGS. 16A, 16B, 17A, 17B, 17C, and 17D illustrate protective efficacy of NanoSTING against interferon (IFN) evasive SARS-CoV-2 Alpha variant (B.1.1.7). Groups of 12 hamsters were each treated with two different doses of NanoSTING (30 μg and 120 μg) and 24 h later challenged with the SARS-CoV-2 Alpha variant (B.1.1.7) (FIG. 16A). Weight changes were monitored daily (FIG. 16B). Animals were euthanized for histopathology (FIGS. 17A and 17B) on day 5, with viral titers of lung and nasal tissues measured on day 2 (n=6) (FIGS. 17C, and 17D). Pathology score (FIG. 17B) and representative hematoxylin and eosin (H&E) images (FIG. 17A) of the lung showed histopathological changes in lungs of Syrian hamsters treated with NanoSTING; all images were acquired at 20×; scale bar, 100 μm. Viral titers were quantified in the lung and nasal tissue by plaque assay on days 2 after the challenge (FIGS. 17C, and 17D). For viral titers, analysis was performed using a Mann-Whitney test. Percent bodyweights were compared via mixed-effects model for repeated measures analysis. Each dot is an individual hamster. Bars and columns show median values. Mann-Whitney test: ****p<0.0001; ***p<0.001; **p<0.01; *p<0.05; ns, not significant.

FIGS. 18A and 18B show prophylactic administration of compositions disclosed herein (e.g., NanoSTING compositions) prior to challenge with influenza A virus. FIG. 18A shows experimental design for a comparison of pre-challenge NanoSTING (40 μg) and pre-challenge Oseltamivir (30 mg/kg/day) treatment followed by challenge with 4× lethal dose 50 (LD50) of sensitive strain (Influenza A/California/04/2009 (H1N1) virus) and monitoring for 14 days (n=10/group) as compared to placebo-treated mice. FIG. 18B shows weight change in subjects from the experiment shown in FIG. 18A. For viral titers, analysis was performed using a Mann-Whitney test. Weight data was compared via mixed-effects model for repeated measures analysis. We compared survival percentages using the Log-Rank Test (Mantel-Cox). Each dot is an individual mice. Bars and columns show median values. Mann-Whitney test: ****p<0.0001; ***p<0.001; **p<0.01; *p<0.05; ns, not significant.

FIGS. 19A, 19B, and 19C show prophylactic administration of compositions disclosed herein (e.g., NanoSTING compositions) prior to challenge with influenza A virus. FIG. 19A shows experimental design for a comparison of pre-challenge NanoSTING (40 μg) and pre-challenge Oseltamivir (30 mg/kg/day) followed by challenge with 4× lethal dose 50 (LD50) resistant strain Influenza A/Hong Kong/2369/2009 (H1N1) and monitoring for 14 days (n=10/group) as compared to unchallenged mice (n=10/group). FIG. 19B shows weight change in subjects from the experiment shown in FIG. 19A. FIG. 19C shows a Meyer Kaplan graph of subject survival during the experiment shown in FIG. 19A. For viral titers, analysis was performed using a Mann-Whitney test. Weight data was compared via mixed-effects model for repeated measures analysis. Survival percentages using the Log-Rank Test (Mantel-Cox) were compared. Each dot is an individual mice. Bars and columns show median values. Mann-Whitney test: ****p<0.0001; ***p<0.001; **p<0.01; *p<0.05; ns, not significant.

FIGS. 20A, 20B, and 20C show prophylactic administration of compositions disclosed herein (e.g., NanoSTING compositions) prior to challenge with influenza A virus. FIG. 20A shows experimental design for a comparison of pre-challenge NanoSTING (40 μg) and pre-challenge Oseltamivir (30 mg/kg/day) followed by challenge with 4× lethal dose 50 (LD50) resistant strain Influenza A/Hong Kong/2369/2009 (H1N1) and monitoring for 7 days (n=10/group) as compared to unchallenged mice (n=10/group). FIG. 20B shows weight change in subjects from the experiment shown in FIG. 20A. FIG. 20C shows infectious viral titers measured by plaque assay in lungs seven days after challenge during the experiment shown in FIG. 20A. For viral titers, analysis was performed using a Mann-Whitney test. Weight data was compared via mixed-effects model for repeated measures analysis. Survival percentages using the Log-Rank Test (Mantel-Cox) were compared. Each dot is an individual mice. Bars and columns show median values. Mann-Whitney test: ****p<0.0001; ***p<0.001; **p<0.01; *p<0.05; ns, not significant.

FIGS. 21A and 21B show prophylactic administration of compositions disclosed herein (e.g., NanoSTING compositions) prior to challenge with influenza A virus. FIG. 21A shows experimental design for a comparison of pre-challenge NanoSTING (40 μg) and pre-challenge Oseltamivir (30 mg/kg/day) followed by challenge with 4× lethal dose 50 (LD50) resistant strain Influenza A/California/04/2009 (H1N1) and monitoring for 14 days (n=10/group) as compared to unchallenged mice (placebo) (n=10/group). FIG. 21B shows weight change in subjects from the experiment shown in FIG. 21A. Weight data was compared via mixed-effects model for repeated measures analysis. Each dot is an individual hamster, and bars indicate median values. Mann-Whitney test: ****p<0.0001; ***p<0.001; **p<0.01; *p<0.05; ns, not significant.

FIGS. 22A, 22B, 22C, and 22D show intranasal administration of NanoSTING limits transmission and viral replication in the nasal passage of contact hamsters exposed to the SARS-CoV-2 Omicron (B.1.1.529) variant. FIG. 22A shows experimental design wherein groups of eight hamsters each on day 0 with 10⁴ TCID50 of SARS-CoV-2 Omicron variant (B.1.1.529) and after 24 h cohoused index hamsters in pairs with contact hamsters (n=8) for 4 days in clean cages. In group II, hamsters were prophylactically pre-treated with 120 μg of NanoSTING 24 h prior to infection, and in group III, contact hamsters were treated with NanoSTING 12 h after the cohousing period began. Contact and index hamsters were euthanized on day 4 of cohousing. Infectious viral particles in the nasal tissues at day 2 and day 5 after viral administration post-infection were measured by plaque assay (FIG. 21B and FIG. 22C). FIG. 22D shows longitudinal measurements of the bodyweight of index hamsters intranasally infected with SARS-CoV-2 Omicron variant. For viral titers, analysis was performed using a Mann-Whitney test. Bars show median values. Mann-Whitney test: ****p<0.0001; ***p<0.001; **p<0.01; *p<0.05; ns, not significant. Weight data was compared via mixed-effects model for repeated measures analysis. Each dot is an individual hamster, and bars indicate median values. Mann-Whitney test: ****p<0.0001; ***p<0.001; **p<0.01; *p<0.05; ns, not significant.

FIGS. 23A and 23B show schematics for NanoSTING-S compositions in accordance with embodiments disclosed herein and an immunogenicity data timeline in accordance with embodiments disclosed herein, respectively.

FIGS. 24A, 24B, 24C, 24D, 24E, 24F, 24G, and 24H show results of NanoSTING-S compositions used in mice, in accordance with embodiments. Lipid NanoSTING-S was used at 10 μg S protein and 20 μg NanoSTING. This leads to cross-reactive responses against multiple strains of SARS-CoV-2 including the Omicron variant.

FIGS. 25A, 25B, 25C, and 25D show the impact of the NanoSTING-S vaccine in the hamster challenge model. NanoSTING-S: 20 μg of S protein and 40 μg of NanoSTING. Weight loss and infectious viral titers in the lung and nasal tissue are shown. As shown, weight loss and infectious viral titer data imply favorable results in the use of compositions comprising NanoSTING-S to accomplish immunity in humans. In some cases, a dose in humans would be 50-200 μg of S protein and 200-500 μg of NanoSTING.

FIGS. 26A, 26B, 26C, 26D, 26E, 26F, 26G, 26H, 26-I, 26J, 26K, 26L, 26M, 26N and 26-O show the impact of the NanoSTING-N vaccine in mice. These support the use of NanoSTING-N to accomplish immunity in humans relying on both antibody responses and T-cell responses. The anticipated dose in humans would be 50-200 μg of N protein and 200-500 μg of NanoSTING.

FIGS. 26P and 26Q show IRF responses of THP-1 dual cells to poly(dA:dT)/LV and MTT assay on THP1-dual cells, respectively. Dose-response NanoSTING with triton X-100 as cytotoxicity control, in accordance with embodiments

FIGS. 27A, 27B, 27C, 27D, 27E, 27F, and 27G show NanoSTING-NS vaccine (20 μg N protein, 10 μg S protein and 20 μg NanoSTING comprising liposomal particle and cGAMP modulator) in mice. The ratio of N protein to S protein was optimized at 2:1 mass ratio. Ratios of N:S of at least 1:1 (mass:mass) or higher provide a balance of immune response against both proteins. This leads to cross-reactive responses against multiple strains of SARS-CoV-2.

FIGS. 28A, 28B, 28C, and 28D show the impact of the NanoSTING-NS (NanoSTING-NS: 30 μg of N protein, 20 μg of S protein and 40 μg of NanoSTING comprising liposomal particle and cGAMP modulator) vaccine in the hamster challenge model. Weight loss and infectious viral titers in the lung and nasal tissue are shown. These support the use of NanoSTING-NS to accomplish sterilizing immunity in humans. The anticipated dose in humans would be 50-200 μg of N protein, 50-200 μg of S protein and 200-500 μg of NanoSTING.

FIGS. 29A and 29B show the impact of the NanoSTING-N vaccine (30 μg of N protein and 40 μg of NanoSTING comprising liposomal particle and cGAMP modulator) in the hamster challenge model. Weight loss and infectious viral titers in the lung and nasal tissue are shown in FIGS. 29C and 29D.

### SUMMARY

The SARS-CoV-2 pandemic has spread at an alarming rate and has highlighted that the existing therapeutic arsenal against RNA viruses is woefully inadequate. Vaccines are the preferred means of protection against SARS-CoV-2 but they suffer from three drawbacks. First, while the current generation of vaccines was developed at remarkable speed (˜10 months), even this rate of development is still slow and vaccines need to be custom manufactured for every emerging virus. Second, the mutational plasticity of RNA viruses like SARS-CoV-2 facilitates their evolution, and newer variants with immune escape potential have emerged. This, in turn, necessitates booster shots for complete protection from disease, even as the entire human population is not yet fully vaccinated. As the human experience with influenza has illustrated, requiring additional booster shots reduces human compliance that in turn facilitates the spread of disease. Third, despite the efficacy of the current vaccines in preventing disease, they do not prevent transmission. The evolution of the SARS-CoV-2 Omicron (B.1.1.529) variant shows that viruses can quickly adapt to facilitate rapid transmission. Thus, while vaccines are necessary, they are not sufficient to fight RNA viruses, and the availability of pre- or post-exposure prophylactics that can both prevent disease and reduce transmission is an urgent and unmet clinical need. As disclosed herein, a single dose of intranasal NanoSTING can work as prophylactic against multiple respiratory viruses (and treatment-resistant variants).

Monoclonal antibodies, like vaccines, can have high efficacy against preventing disease but suffer from all of the same disadvantages of vaccines listed above. The SARS-CoV-2 Omicron variant is almost completely resistant to neutralization by antibodies. Additionally, monoclonal antibodies are expensive therapeutics and are administered in a clinical setting, further limiting their widespread use. If only efficacy is compared, prophylactic administration of antibodies (12 h before challenge) in hamsters led to protection from clinical disease (˜2-5% weight loss and ˜300-fold reduction in viral titers in the lung) with no impact on transmission. Compositions disclosed herein (e.g., NanoSTING compositions) are easy to administer (e.g., intranasally) and provide a broader window of administration (24-72 h), with comparable efficacy in reducing clinical disease while also reducing transmission.

Oral antivirals that directly inhibit one or more viral proteins have been developed against SARS-CoV-2 (e.g. Molnupiravir and Paxlovid) and tested in humans, but they are susceptible to viral evolution and resistance. Furthermore, oral antivirals are designed as post-exposure prophylactics to prevent clinical disease and have no impact on viral transmission. In contrast to these pathogen-specific antivirals, compositions disclosed herein (e.g., NanoSTING compositions) work against multiple respiratory viruses. In addition, the favorable efficacy profile comparing compositions disclosed herein to these antivirals in small animal models, and the fact that these antivirals have had efficacy in humans (30-89% in reducing clinical disease with SARS-CoV-2), argues well for the clinical translational potential of the compositions disclosed herein (e.g., NanoSTING compositions).

Immunomodulators including defective viral genome particles, cytokines, and small molecule agonists have been tested as antivirals. Defective interfering particles (DIPs) have incomplete genomes and when administered therapeutically inhibit replication of the wild-type virus. Although these particles have demonstrated efficacy for both SARS-CoV-2 and influenza in mitigating disease in small animal models, the DIPs have to be generated for each virus individually. Defective viral genomes (DVGs) based on the poliovirus induced a broad IFN-I response and were protective against multiple viruses. Limited replication was essential for the efficacy of the DVGs but their broad applicability is limited by concerns of both safety and the presence of pre-existing antibodies in vaccinated people. Lipid nanoparticles complexed with the defective genomes can mitigate these concerns and have shown efficacy against SARS-CoV-2 variants in K18-hACE2 mice. However, the generalizability of this approach in the absence of viral replication to other viruses has not been demonstrated.

Direct administration of aerosolized interferons to engage antiviral innate immunity has been tested both in animals and humans. In hamsters challenged with SARS-CoV-2, prophylactic or early administration of universal interferon reduces lung damage, provides moderate protection against weight loss (10% vs 20% for untreated animals), and reduces infectious viral particles (100-fold). In humans, post-exposure prophylaxis with nebulized IFN-α2b was associated with lower in-hospital mortality in comparison with no administration of IFN-α2b. By contrast, administration of IFN-α2b more than five days after admission delayed recovery and increased mortality, suggesting that the timing of IFN-α2b is essential for benefit. The limited impact of IFN-α for COVID19 mirrors its minor efficacy as a prophylactic against influenza in humans.

Other synthetic small molecule agonists of pattern recognition receptors (PRRs) including stem-loop RNA 14 (SLR14), a minimal RIG-I agonist; and STING agonist, diAbzl, have been tested against SARS-CoV-2 in K18-hACE2 mice. As with all small-molecule drugs their safety, off-target activity, and pharmacokinetics need to be thoroughly evaluated before translation. In some cases, compositions disclosed herein (e.g., some NanoSTING compositions disclosed herein) are comprised of naturally occurring lipids that have already been tested in humans and cGAMP, the immunotransmitter of danger signals that is conserved across mammals including humans. These compositions can lead to safe and sustained delivery and, consequently, can function as broad-spectrum antivirals.

This disclosure shows that compositions disclosed herein (e.g., NanoSTING compositions) are a safe, stable, low-cost, and effective antiviral that engages the STING pathway to initiate a broad-spectrum antiviral response effective against multiple respiratory pathogens. The advantage of using the natural immunotransmitter, cGAMP is that STING activation can lead to both interferon dependent and interferon independent activities to control viral replication. The broad spectrum of compositions of the present disclosure (e.g., NanoSTING compositions) ensures that the activity works against multiple variants including variants resistant to other antivirals. The ability to engage adaptive immunity ensures that treatment with compositions of the disclosure (e.g., NanoSTING compositions) also protects from reinfection and leads to durable immunity. The availability of broad-spectrum drugs that can be stored easily for long periods ensures preparation to tackle future respiratory viruses as they emerge.

Disclosed herein are compositions, methods, kits, and systems for the treatment of subjects in need thereof, such as subjects having pathogens capable of causing respiratory diseases. In some cases, compositions, methods, kits, and/or systems disclosed herein can be useful in preventing a subject (e.g., an uninfected subject) from becoming infected with a pathogen and/or contracting a disease that the pathogen is capable of causing, for example, by preventing infection of the subject with the pathogen or by preventing progression (e.g., spread or increase in severity) of a condition (e.g., a respiratory disease) caused by the pathogen in the subject. In some cases, compositions, methods, kits, and/or systems disclosed herein can be useful in preventing reinfection of a subject with a pathogen (e.g., wherein the pathogen is capable of causing a respiratory disease). In some cases, compositions, methods, kits, and/or systems disclosed herein can be useful in preventing transmission of a pathogen (e.g., a pathogen capable of causing a respiratory disease) from a first (e.g., infected) subject to a second (e.g., uninfected) subject. In some cases, the pathogen is a virus. In some cases, the pathogen is a coronavirus. In some cases, the pathogen is an influenza virus.

In various embodiments, a composition comprising: a lipid-based particle and a modulator, wherein the modulator is a pattern recognition receptor agonist, an activator of the immune system, or a combination thereof. In various embodiments, a composition for use in reducing a risk of contracting a respiratory disease in a subject after exposure of the subject to a pathogen capable of causing the respiratory disease, comprises: a lipid-based particle and a modulator, wherein the modulator is a pattern recognition receptor agonist, an activator of the immune system, or a combination thereof. In various embodiments, a composition for use in reducing a risk of contracting a respiratory disease in a subject before exposure of the subject to a pathogen capable of causing the respiratory disease comprises a lipid-based particle and a modulator, wherein the modulator is a pattern recognition receptor agonist, an activator of the immune system, or a combination thereof. In various embodiments composition for use in treating a respiratory disease in a subject comprises: a lipid-based particle and a modulator, wherein the modulator is a pattern recognition receptor agonist, an activator of the immune system, or a combination thereof. In various embodiments, a composition for use in reducing a rate of progression of a respiratory disease in a subject having the respiratory disease comprises: a lipid-based particle and a modulator, wherein the modulator is a pattern recognition receptor agonist, an activator of the immune system, or a combination thereof. In various embodiments, a composition for use in reducing a risk of transmission of a respiratory disease from a subject having the respiratory disease to a subject not having the respiratory disease comprises: a lipid-based particle and a modulator, wherein the modulator is a pattern recognition receptor agonist, an activator of the immune system, or a combination thereof. In some cases, the modulator is encapsulated within the lipid-based particle. In some cases, the subject has previously contracted the respiratory disease. In some cases, the lipid-based particle comprises an antigen. In some cases, the lipid-based particle comprises a first antigen and a second antigen. In some cases, the first antigen is a spike protein molecule or portion thereof. In some cases, the second antigen is a nucleocapsid protein molecule or portion thereof. In some cases, the lipid-based particle comprises a greater quantity of nucleocapsid protein molecules than spike protein molecules. In some cases, the ratio of nucleocapsid protein molecules to spike protein molecules is at least 1:1. In some cases, the composition is formulated for intranasal delivery. In some cases, the modulator is an agonist of the STING pathway. In some cases, the lipid-based particle comprises DPPC, DPPG, cholesterol, and DPPE-PEG2000 in a 10:1:1:1 ratio. In some cases, the antigen is associated with an outer surface of the lipid-based particle. In some cases, the disease comprises an infection caused by a pathogen. In some cases, the pathogen is a respiratory pathogen. In some cases, the respiratory pathogen is a virus. In some cases, the virus is selected from an influenza virus, a parainfluenza virus, an adenovirus, an enterovirus, a coronavirus, a respiratory syncytial virus, a rhinovirus, a DNA virus, an RNA virus, a variant thereof, or a combination thereof. In some cases, the virus is an influenza virus. In some cases, the influenza virus comprises an oseltamivir-sensitive strain, a treatment resistant strain, or a combination thereof. In some cases, the virus is a coronavirus. In some cases, the coronavirus is a SARS-CoV-2 virus, an alpha variant thereof, a delta variant thereof, an omicron variant thereof, or combinations thereof. In some cases, the composition is lyophilized. In some cases, the composition is in liquid form. In some cases, the modulator is selected from the group consisting of bis-(3′,5′)-cyclic dimeric guanosine monophosphate (c-di-GMP), cyclic guanosine monophosphate-adenosine monophosphate (cGAMP), amidobenzimidazole, derivatives of amidobenzimidazole, nucleotide modulators, plasmid DNA modulators, divalent cations, CF501, SHR1032, or combinations thereof. In some cases, the modulator comprises cyclic guanosine monophosphate-adenosine monophosphate (cGAMP). In various aspects, a kit comprises a composition of any one of the embodiments or aspects disclosed herein, including instructions for use. In various aspects, a composition of any of the embodiments or aspects disclosed herein is used in the manufacture of a medicament for treating a respiratory disease.

In various aspects, a method comprises: administering a composition according to any of the embodiments or aspects disclosed herein to the subject, wherein the subject does not exhibit symptoms of the respiratory disease. In some cases, a sample obtained from the subject has a detectable level of a pathogen associated with the respiratory disease. In some cases, a sample obtained from the subject does not have a detectable level of a pathogen associated with the respiratory disease. In various aspects, a method comprises: administering a compound according to any of the embodiments or aspects disclosed herein to the subject, wherein the subject exhibits symptoms of the respiratory disease. In some cases, a sample obtained from the subject has a detectable level of a pathogen associated with the respiratory disease. In some cases, the composition is administered in at least one dose. In some cases, the composition is administered in one dose. In some cases, the composition is administered in two doses. In some cases, the composition is administered to the subject before the subject is exposed to a pathogen associated with the respiratory disease. In some cases, the composition is administered to the subject at least one day before the subject is exposed to a pathogen associated with the respiratory disease. In some cases, the composition is administered to the subject at least three days before the subject is exposed to the pathogen associated with the respiratory disease. In some cases, the composition is administered through intranasal administration. In some cases, the composition is administered through inhalational administration. In some cases, the method is used to prevent the establishment of the disease in the subject. In some cases, the method is used to prevent progression of the disease in the subject. In some cases, the method is used to prevent the transmission of disease to a second subject. In some cases, the method initiates an innate immune response that leads to associated adaptive immunity.

### DETAILED DESCRIPTION

Therapeutics and vaccines for respiratory diseases have numerous limitations. For instance, vaccines provide short-term relief from SARS-CoV2. However, rapid evolution of resistant viral variants necessitates additional supportive strategies, including broad-spectrum antiviral agents with prophylactic and therapeutic properties.

SARS-CoV2 and influenza are one of the leading infectious diseases causing mortality and morbidity worldwide. Drug resistance and viral mutations are the leading challenges in influenza prevention and treatment. The development of off-the-shelf, effective, safe, and low-cost drugs for prophylaxis against respiratory viral infections is a major unmet medical need. Disclosed herein are compositions and methods for the prevention and/or treatment of a disease or pathological condition of a subject (e.g., an animal, such as a mammal), such as a respiratory disease. In some embodiments, preventing a disease or pathological condition comprises preventing the establishment of the disease or the pathological condition in a subject. In some embodiments, preventing a disease or pathological condition comprises preventing the establishment of the disease or the pathological condition in a subject (e.g., after exposure of the subject to a pathogen). In some embodiments, preventing a disease or pathological condition comprises preventing the development (e.g., progression) of the disease or the pathological condition (e.g., from a first state to a second, more severe or progressed state) in a subject (e.g., after establishment of a disease in the subject, for example, via infection from a pathogen). In some embodiments, preventing a disease or pathological condition comprises preventing the transmission of the disease or the pathological condition from a first subject to a second subject.

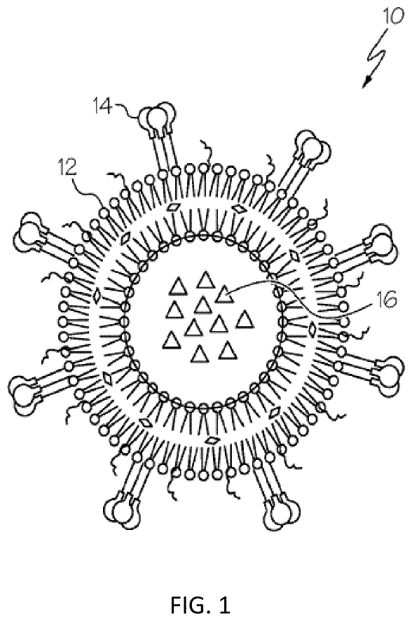

A composition 10 useful for preventing or treating a disease or pathological condition of a subject can comprise a particle 12 (e.g., a liposome described herein), a modulator 16 (e.g., a STING agonist, such as cGAMP), and/or an antigen 14 (e.g., as shown in FIG. 1). In some cases, a composition described herein can comprise a modulator (e.g., a STING agonist, such as cGAMP) without a particle or antigen. In some cases, a composition described herein can comprise a modulator (e.g., a STING agonist, such as cGAMP) encapsulated within, in contact with, bound to, and/or not in contact with but delivered (e.g., intranasally with) a particle described herein. In some cases, encapsulating one or more modulators within a particle (or, in some cases, binding the one or more modulators to or incorporating the one or more modulators into the particle) as described herein can increase uptake efficiency, tissue targeting, stability, and/or efficacy of the composition or a method of its use, e.g., by controlling (e.g., maintaining) spatial concentration of the one or more modulators and/or by presenting a recognition signal (e.g., an antigen) to a target cell. In some cases, compositions comprising one or more modulators without a particle or antigen (or comprising one or more modulators outside of and unassociated with a particle of the composition) can elicit a more immediate response from a target tissue or cell and/or decrease the cost and/or complexity of manufacture of the composition. In some cases, a composition described herein can comprise one or more modulators (e.g., one or more STING agonist molecules, such as one or more cGAMP molecules) encapsulated within a particle (e.g., liposome) and one or more modulators (e.g., one or more STING agonist molecules, such as one or more cGAMP molecules) delivered to a subject with but not encapsulated by, bound to, or incorporated in the particle. In some cases, a composition comprising one or more modulators (e.g., one or more STING agonist molecules, such as one or more cGAMP molecules) encapsulated within a particle (e.g., liposome) and one or more modulators (e.g., one or more STING agonist molecules, such as one or more cGAMP molecules) delivered to a subject with but not encapsulated by, bound to, or incorporated in the particle can provide benefits of both “naked” modulators (e.g., more immediate response and/or different target cells or tissues than encapsulated modulators) and modulator(s) encapsulated, bound to, or incorporated within a particle (e.g., liposome) described herein. In some cases, compositions described herein can comprise a lipid-based liposome particle, modulator (such as a pattern recognition receptor agonist, e.g., a STING agonist), and an application-specific antigen (such as a coronavirus spike protein (“S-protein”), a nucleocapsid protein (“N-protein”), or a chimeric antigen protein) associated with the liposome particle. In some embodiments, association of an application-specific antigen with (e.g., an outer surface of) a liposome particle encapsulating a modulator payload can significantly increase the targeting and/or delivery of the modulator payload to a target tissue of interest (e.g., an intranasal compartment and/or a lung compartment of a subject). In some embodiments, association (e.g., direct association, such as through incorporation into the liposomal membrane or through coupling to the liposomal membrane surface) of an application-specific antigen with a liposome particle may increase the efficacy of the composition in preventing and/or treating a disease or pathological condition in a subject, for example, by spatially concentrating an antigen and a modulator of the composition (e.g., at a target tissue). Disclosed herein are, compositions (e.g., NanoSTING compositions, which can be liposomally encapsulated immunotransmitters) that can rapidly activate the body's innate immune system to facilitate a broad-spectrum antiviral response against influenza and SARS-CoV-2 variants in mice and hamsters. In some cases, a single intranasal dose of NanoSTING can: (1) treat infections throughout the respiratory tree and minimize symptoms, (2) reduce transmission by decreasing infectious virus in the nasal passage, (3) protect against oseltamivir-resistant influenza and highly infectious strains of SARS-CoV-2 (alpha and delta), and/or (4) provide durable protection against reinfection by stimulating adaptive immunity. In some cases, administration of NanoSTING can upregulate interferon-stimulated pathways and/or antiviral pathways, e.g., in nasal turbinates and/or lungs. In some embodiments, NanoSTING compositions can be stable, low-cost, broad-spectrum antiviral therapeutics for prophylactic or early post-exposure treatment.

In some cases, compositions and methods disclosed herein (e.g., comprising NanoSTING, which can comprise a nanoparticle formulation of cGAMP) can enable sustained release of cGAMP to both the nasal compartment and the lung over a period of 48 hours or more following administration to a subject. Compositions disclosed herein (e.g., comprising NanoSTING formulations) can be comprised of lipids and a STING activator like cGAMP but can also include one or more divalent metal ions, such as Mg²⁺ or Mn²⁺. The cGAMP (e.g., delivered to a subject in accordance with compositions and or methods disclosed herein) can activate multiple antiviral pathways and facilitates type-I interferon (IFN-I) mediated response. Quantitative modeling using SARS-CoV-2 infections in humans can be used to confirm that pre-exposure or early post-exposure prophylaxis with even small amounts of compositions disclosed herein (e.g., NanoSTING compositions) can yield clinically observable benefit. Compositions disclosed herein (e.g., NanoSTING) can be used to prevent clinical disease (e.g., SARS-CoV-2 and/or multiple variants of influenza A, including oseltamivir resistant influenza A), reduce viral loads (e.g., by several orders of magnitude), reduce pathogen and/or disease transmission, and/or engage adaptive immunity to confer durable protection from reinfection. The stability and ease of administration, and the comprehensive nature of the immune response elicited make compositions disclosed herein (e.g., NanoSTING) provide ideal non-invasive and broad-spectrum antiviral therapeutic compositions against respiratory viruses.

### Compositions

A composition (e.g., a therapeutic composition) for preventing or treating a disease or pathological condition in a subject can comprise a delivery vehicle (e.g., a particle). A delivery vehicle of a composition useful in preventing or treating a disease or pathological condition in a subject can be a particle, such as a lipid-based particle. For example, a composition useful in preventing or treating a disease or pathological condition in a subject can comprise a lipid-based particle, such as a liposome (e.g., having a membrane with an outer surface and an interior space). In some embodiments, the composition comprises one or more modulators (e.g., one or more types of modulators). In some embodiments, a modulator can comprise a small molecule, a protein or fragment thereof, and/or a polynucleotide or fragment thereof. In some embodiments, a modulator (e.g., a STING agonist) or combination of modulators can be selected for use in a composition described herein for its ability to induce activation or inhibition of a signaling pathway and/or a systemic response pathway (e.g., the stimulator of interferon genes (STING) pathway) in a subject. A composition useful in preventing or treating a disease or pathological condition in a subject (or transmission thereof to a second subject) can comprise one or more antigens. A composition useful in preventing or treating a disease or pathological condition in a subject (or transmission thereof to a second subject) can comprise a plurality of antigens. In some embodiments, an antigen of a composition described herein can elicit an immune response in a subject, which may be useful in preventing or abrogating a disease (e.g., a respiratory disease) or pathological condition (e.g., cancer) in the subject. In some embodiments, a composition for preventing or treating a disease or a pathological condition in a subject can comprise a modulator (e.g., a STING agonist, such as cGAMP) encapsulated within a particle (e.g., a liposome) wherein an antigen is associated with the liposome or a portion thereof (e.g., via incorporation into the liposomal membrane).

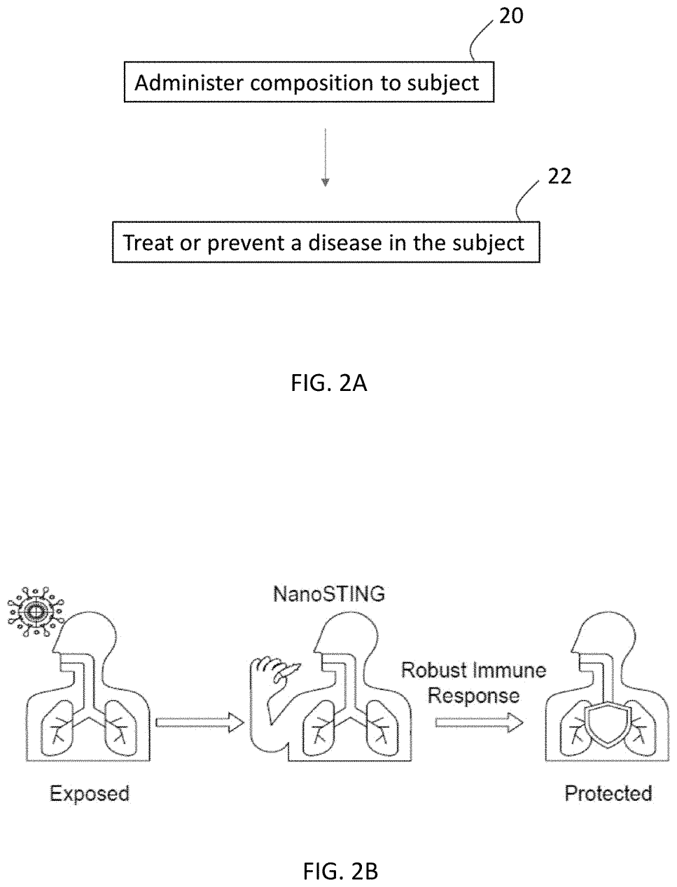

In some embodiments, a composition described herein can be used in a method for treating or preventing a disease or pathological condition in a subject (e.g., a mammal, such as a human). In some embodiments, a composition described herein can be administered to a subject before exposure to a pathogen (e.g., a pathogen causing a respiratory disease or cancer), for example, to prevent the subject from acquiring a disease or condition associated with exposure to (e.g., infection by) the pathogen (e.g., as illustrated in FIG. 2A). In some embodiments, a composition disclosed herein can be administered in the form of a vaccine. In some embodiments, a composition described herein can be administered to a subject after exposure to a pathogen (e.g., a pathogen causing a respiratory disease or cancer), for example, to treat (e.g., ameliorate or, In some embodiments, cure) a disease or condition associated with exposure to (e.g., infection by) the pathogen, for example, after the subject has acquired a disease or condition associated with the pathogen from exposure to the pathogen (e.g., as illustrated in FIG. 2B).

### Particles

A composition of the present disclosure can comprise a delivery vehicle, such as a particle. A particle can comprise a means of conveying a payload (e.g., a modulator payload) and/or one or more antigens (e.g., an antigen associated with a portion of the particle, such as a membrane or outer surface). In some embodiments, a particle can comprise a membrane or wall. In some embodiments, a membrane or wall of a particle can define an interior space. In some embodiments, an interior space of a particle can comprise one or more modulators. In some embodiments, one or more antigens can be associated with a membrane or wall of the particle (e.g., an outer surface of a membrane or wall of the particle). A particle can comprise a lipid-based particle, a carbon-based particle, a metal-based particle, or combinations thereof.

A particle of a composition described herein can be a lipid-based particle. In some embodiments, the lipid-based particle can be a liposome. A composition described herein can comprise a pulmonary surfactant-biomimetic particle. A particle of a composition disclosed herein (e.g., a lipid-based particle) can comprise a plurality of molecules, for example, assembled into a membrane. In some embodiments, a particle (e.g., a lipid-based particle) or a portion thereof can be anionic (e.g., can comprise an anionic membrane or one or more anionic lipids). For example, a particle (e.g., a lipid-based particle) can comprise an anionic lipid (e.g., DPPE, DPPE-PEG2000, DPPC, DPPG) or a neutral lipid (e.g., cholesterol). In some embodiments, a particle of a composition described herein (e.g., NanoSTING compositions comprising a lipid-based nanoparticle) may comprise no cationic lipids. In some embodiments, a particle (e.g., a lipid-based particle) or a portion thereof can be cationic. In some cases, a particle (e.g., a lipid-based particle) can comprise a cationic (e.g., positively charged) lipid. For example, a particle (e.g., a lipid-based particle) can comprise a lipid selected from 1,2-dipalmitoyl-3-trimethylammonium-propane chloride (DPTAP) or 1,2-dioleoyl-3-trimethylammonium propane (DOTAP). In some embodiments, a particle (e.g., a lipid-based particle) can be zwitterionic. In some embodiments, a particle (e.g., a lipid-based particle) or a portion thereof can have a net zero charge. In some embodiments, a particle (e.g., a lipid-based particle) or a portion thereof can be uncharged. In some embodiments, a particle (e.g., liposome) of a composition described herein can comprise dipalmitoylphosphatidylcholine, dipalymitoylphosphatidylglycerol, 1,2-bis(diphenylphosphino)ethane (DPPE), cholesterol, or a combination thereof. In some embodiments, a particle (e.g., liposome) of a composition described herein can comprise a poly(ethyleneglycol)-lipid (e.g., a PEG-lipid). In some embodiments, a particle (e.g., liposome) of a composition described herein can comprise DPPC (1,2-dipalmitoyl-sn-glycero-3-phosphocholine), DPPG (1,2-dipalmitoyl-sn-glycero-3-phospho-(1′-rac-glycerol)), DPPE-PEG2000 (1,2-dipalmitoyl-sn-glycero-3-phosphoethanolamine-N-[methoxy(polyethylene glycol)-2000]), cholesterol, 1,2-dipalmitoyl-3-trimethylammonium-propane chloride (DPTAP), 1,2-dioleoyl-3-trimethylammonium propane (DOTAP), or a combination thereof. In some embodiments, a particle can comprise a combination of DPPC and DPPG, for example, at a molar ratio of about 10:1. In some embodiments, a particle can comprise a combination of DPPC and cholesterol, for example, at a molar ratio of about 10:1. In some embodiments, a particle can comprise a combination of DPPC and DPPE-2000, for example, at a molar ratio of about 10:1. In some embodiments, a particle can comprise a combination of DPPG and cholesterol, for example, at a molar ratio of about 1:1. In some embodiments, a particle can comprise a combination of cholesterol and DPPE-PEG2000, for example, at a molar ratio of about 1:1. In some embodiments, a particle can comprise a combination of DPPG and DPPE-PEG2000, for example, at a molar ratio of about 1:1. In some embodiments, a particle can comprise DPPC, DPPG, cholesterol, and DPPE-PEG2000. In some embodiments, a particle can be composed of a molar ratio of 10:1:1:1 of DPPC, DPPG, cholesterol, and DPPE-PEG2000, respectively. In some embodiments, a particle can be composed of a molar ratio of 20:1:1:1 of DPPC, DPPG, cholesterol, and DPPE-PEG2000, respectively. In some embodiments, a particle can be composed of a molar ratio of 5:1:1:1 of DPPC, DPPG, cholesterol, and DPPE-PEG2000, respectively. In some embodiments, a particle can be composed of a molar ratio of 10:2:1:1 of DPPC, DPPG, cholesterol, and DPPE-PEG2000, respectively. In some embodiments, a particle can be composed of a molar ratio of 10:1:2:1 of DPPC, DPPG, cholesterol, and DPPE-PEG2000, respectively. In some embodiments, a particle can be composed of a molar ratio of 10:1:1:2 of DPPC, DPPG, cholesterol, and DPPE-PEG2000, respectively. In some embodiments, a particle can be composed of a molar ratio of 10:2:2:1 of DPPC, DPPG, cholesterol, and DPPE-PEG2000, respectively. In some embodiments, a particle can be composed of a molar ratio of 10:1:2:2 of DPPC, DPPG, cholesterol, and DPPE-PEG2000, respectively. In some embodiments, a particle can be composed of a molar ratio of 10:2:1:2 of DPPC, DPPG, cholesterol, and DPPE-PEG2000, respectively. In some embodiments, a molar ratio of a first molecule comprising a particle (e.g., a lipid-based particle) to a second molecule comprising the particle can be from 1:100 to 1:1, from 1:50 to 1:2, from 1:25 to 1:3, from 1:10 to 1:5, from 1:50 to 1:1, from 1:25 to 1:1, from 1:10 to 1:1, from 1:5 to 1:1, from 1:3 to 1:1, from 1:25 to 1:10, from 1:50 to 1:25, from 1:100 to 1:50, or greater than 1:100. In some embodiments, a molar ratio of a first molecule comprising a particle (e.g., a lipid-based particle) of the present disclosure to a second molecule comprising the particle can be 1:1, 1:2, 1:3, 1:4, 1:5, 1:10, 1:15, 1:20, 1:25, 1:50, 1:100, or any range therebetween. In some embodiments, a molar ratio of a first molecule comprising a particle (e.g., a lipid-based particle) can be 1:5. In some embodiments, a molar ratio of a first molecule comprising a particle (e.g., a lipid-based particle) can be 1:10. In some embodiments, a molar ratio of a first molecule comprising a particle (e.g., a lipid-based particle) can be 1:20. In some embodiments, a molar ratio of a first molecule comprising a particle (e.g., a lipid-based particle) can be 1:50. In some embodiments, a first molecule comprising a particle (e.g., a lipid-based particle) can be 1:100. In some embodiments, a first molecule comprising a particle (e.g., a lipid-based particle) of a composition described herein can be dipalmitoylphosphatidylcholine, dipalymitoylphosphatidylglycerol, 1,2-bis(diphenylphosphino)ethane (DPPE), cholesterol, or a combination thereof. In some embodiments, a second molecule comprising a particle (e.g., lipid-based particle) of a composition described herein can be DPPC (1,2-dipalmitoyl-sn-glycero-3-phosphocholine), DPPG (1,2-dipalmitoyl-sn-glycero-3-phospho-(1′-rac-glycerol)), DPPE-PEG2000 (1,2-dipalmitoyl-sn-glycero-3-phosphoethanolamine-N-[methoxy(polyethylene glycol)-2000]), or cholesterol.

A particle of a composition described herein can be a nanoparticle. Maintaining a small particle diameter (e.g., mean hydrodynamic particle diameter less than 300 nanometers (nm), less than 200 nm, less than 150 nm, less than 120 nm, less than 115 nm, less than 111 nm, less than 110 nm, less than 105 nm, less than 100 nm, less than 95 nm, less than 90 nm, less than 85 nm, or less than 80 nm) and/or low polydispersity (e.g., polydispersity index less than 0.25, less than 0.24, less than 0.23, less than 0.22, less than 0.21, or less than 0.20) can improve particle stability and/or efficiency of delivery. In some embodiments, a particle can have an outer diameter of 1 nanometer to 500 nanometers, 1 nanometer to 750 nanometers, or 1 nanometer to 1,000 nanometers. In some embodiments, a particle can have an outer diameter of 1 nanometer to 10 nanometers, 1 nanometer to 15 nanometers, 1 nanometer to 20 nanometers, 1 nanometer to 30 nanometers, 1 nanometer to 50 nanometers, 1 nanometer to 75 nanometers, 1 nanometer to 100 nanometers, 1 nanometer to 150 nanometers, 1 nanometer to 200 nanometers, 1 nanometer to 250 nanometers, 1 nanometer to 300 nanometers, 1 nanometer to 400 nanometers, 1 nanometer to 500 nanometers, 10 nanometers to 15 nanometers, 10 nanometers to 20 nanometers, 10 nanometers to 30 nanometers, 10 nanometers to 50 nanometers, 10 nanometers to 75 nanometers, 10 nanometers to 100 nanometers, 10 nanometers to 150 nanometers, 10 nanometers to 200 nanometers, 10 nanometers to 250 nanometers, 10 nanometers to 300 nanometers, 15 nanometers to 20 nanometers, 15 nanometers to 30 nanometers, 15 nanometers to 50 nanometers, 15 nanometers to 75 nanometers, 15 nanometers to 100 nanometers, 15 nanometers to 150 nanometers, 15 nanometers to 200 nanometers, 15 nanometers to 250 nanometers, 15 nanometers to 300 nanometers, 20 nanometers to 30 nanometers, 20 nanometers to 50 nanometers, 20 nanometers to 75 nanometers, 20 nanometers to 100 nanometers, 20 nanometers to 150 nanometers, 20 nanometers to 200 nanometers, 20 nanometers to 250 nanometers, 20 nanometers to 300 nanometers, 30 nanometers to 50 nanometers, 30 nanometers to 75 nanometers, 30 nanometers to 100 nanometers, 30 nanometers to 150 nanometers, 30 nanometers to 200 nanometers, 30 nanometers to 250 nanometers, 30 nanometers to 300 nanometers, 50 nanometers to 75 nanometers, 50 nanometers to 100 nanometers, 50 nanometers to 150 nanometers, 50 nanometers to 200 nanometers, 50 nanometers to 250 nanometers, 50 nanometers to 300 nanometers, 75 nanometers to 100 nanometers, 75 nanometers to 150 nanometers, 75 nanometers to 200 nanometers, 75 nanometers to 250 nanometers, 75 nanometers to 300 nanometers, 100 nanometers to 150 nanometers, 100 nanometers to 200 nanometers, 100 nanometers to 250 nanometers, 100 nanometers to 300 nanometers, 150 nanometers to 200 nanometers, 150 nanometers to 250 nanometers, 150 nanometers to 300 nanometers, 200 nanometers to 250 nanometers, 200 nanometers to 300 nanometers, or 250 nanometers to 300 nanometers. In some embodiments, a particle can have an outer diameter of 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, or 500 nanometers. In some embodiments, a particle can have an outer diameter of at least 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, or 500 nanometers. In some embodiments, a particle can have an outer diameter of at most 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, 500 nanometers, 750 nanometers, or 1,000 nanometers.

In some embodiments, a population of particles can have an average (e.g., outer) diameter of 1 nanometer to 1,000 nanometers, for example, for 1 day (e.g., up to 24 hours), 2 days (e.g., up to 48 hours), 3 days (e.g., up to 72 hours), up to 96 hours, from 4 to 7 days, more than 7 days, from 7 to 30 days, or more than 30 days at 25 degrees Celsius (° C.), which can be room temperature. In some embodiments, a population of particles can have an average (e.g., outer) diameter of 1 nanometer to 100 nanometers, for example, for 1 day (e.g., up to 24 hours), 2 days (e.g., up to 48 hours), 3 days (e.g., up to 72 hours), up to 96 hours, from 4 to 7 days, more than 7 days, from 7 to 30 days, or more than 30 days at 25 degrees Celsius (° C.). In some embodiments, a population of particles can have an average (e.g., outer) diameter of greater than 100 nanometers to up to 200 nanometers, for example, for 1 day (e.g., up to 24 hours), 2 days (e.g., up to 48 hours), 3 days (e.g., up to 72 hours), up to 96 hours, from 4 to 7 days, more than 7 days, from 7 to 30 days, or more than 30 days at 25 degrees Celsius (° C.). In some embodiments, a population of particles can have an average (e.g., outer) diameter of greater than 200 nanometers to up to 300 nanometers, for example, 1 day (e.g., up to 24 hours), 2 days (e.g., up to 48 hours), 3 days (e.g., up to 72 hours), up to 96 hours, from 4 to 7 days, more than 7 days, from 7 to 30 days, or more than 30 days at 25 degrees Celsius (° C.). In some embodiments, a population of particles can have an average (e.g., outer) diameter of 1 nanometer to 20 nanometers, 1 nanometer to 30 nanometers, 1 nanometer to 50 nanometers, 1 nanometer to 75 nanometers, 1 nanometer to 100 nanometers, 1 nanometer to 125 nanometers, 1 nanometer to 150 nanometers, 1 nanometer to 175 nanometers, 1 nanometer to 200 nanometers, 1 nanometer to 300 nanometers, 1 nanometer to 1,000 nanometers, 20 nanometers to 30 nanometers, 20 nanometers to 50 nanometers, 20 nanometers to 75 nanometers, 20 nanometers to 100 nanometers, 20 nanometers to 125 nanometers, 20 nanometers to 150 nanometers, 20 nanometers to 175 nanometers, 20 nanometers to 200 nanometers, 20 nanometers to 300 nanometers, 20 nanometers to 1,000 nanometers, 30 nanometers to 50 nanometers, 30 nanometers to 75 nanometers, 30 nanometers to 100 nanometers, 30 nanometers to 125 nanometers, 30 nanometers to 150 nanometers, 30 nanometers to 175 nanometers, 30 nanometers to 200 nanometers, 30 nanometers to 300 nanometers, 30 nanometers to 1,000 nanometers, 50 nanometers to 75 nanometers, 50 nanometers to 100 nanometers, 50 nanometers to 125 nanometers, 50 nanometers to 150 nanometers, 50 nanometers to 175 nanometers, 50 nanometers to 200 nanometers, 50 nanometers to 300 nanometers, 50 nanometers to 1,000 nanometers, 75 nanometers to 100 nanometers, 75 nanometers to 125 nanometers, 75 nanometers to 150 nanometers, 75 nanometers to 175 nanometers, 75 nanometers to 200 nanometers, 75 nanometers to 300 nanometers, 75 nanometers to 1,000 nanometers, 100 nanometers to 125 nanometers, 100 nanometers to 150 nanometers, 100 nanometers to 175 nanometers, 100 nanometers to 200 nanometers, 100 nanometers to 300 nanometers, 100 nanometers to 1,000 nanometers, 125 nanometers to 150 nanometers, 125 nanometers to 175 nanometers, 125 nanometers to 200 nanometers, 125 nanometers to 300 nanometers, 125 nanometers to 1,000 nanometers, 150 nanometers to 175 nanometers, 150 nanometers to 200 nanometers, 150 nanometers to 300 nanometers, 150 nanometers to 1,000 nanometers, 175 nanometers to 200 nanometers, 175 nanometers to 300 nanometers, 175 nanometers to 1,000 nanometers, 200 nanometers to 300 nanometers, 200 nanometers to 1,000 nanometers, or 300 nanometers to 1,000 nanometers, for example, up to 30 days at 25 degrees Celsius (° C.). In some embodiments, a population of particles can have an average (e.g., outer) diameter of 1 nanometer, 20 nanometers, 30 nanometers, 50 nanometers, 75 nanometers, 100 nanometers, 125 nanometers, 150 nanometers, 175 nanometers, 200 nanometers, 300 nanometers, or 1,000 nanometers, for example, up to 30 days at 25 degrees Celsius (° C.). In some embodiments, a population of particles can have an average (e.g., outer) diameter of at least 1 nanometer, 20 nanometers, 30 nanometers, 50 nanometers, 75 nanometers, 100 nanometers, 125 nanometers, 150 nanometers, 175 nanometers, 200 nanometers, 300 nanometers, or 1,000 nanometers, for example, up to 30 days at 25 degrees Celsius (° C.). In some embodiments, a population of particles can have an average (e.g., outer) diameter of at most 1 nanometer, 20 nanometers, 30 nanometers, 50 nanometers, 75 nanometers, 100 nanometers, 125 nanometers, 150 nanometers, 175 nanometers, 200 nanometers, 300 nanometers, or 1,000 nanometers, for example, up to 30 days at 25 degrees Celsius (° C.). In some cases, an average diameter can be a mean hydrodynamic particle diameter.

In some embodiments, a population of particles can have an average (e.g., outer) diameter of 1 nanometer to 1,000 nanometers, for example, for 1 day (e.g., up to 24 hours), 2 days (e.g., up to 48 hours), 3 days (e.g., up to 72 hours), up to 96 hours, from 4 to 7 days, more than 7 days, from 7 to 30 days, or more than 30 days at 37 degrees Celsius (° C.), which can be body temperature. In some embodiments, a population of particles can have an average (e.g., outer) diameter of 1 nanometer to 100 nanometers, for example, for 1 day (e.g., up to 24 hours), 2 days (e.g., up to 48 hours), 3 days (e.g., up to 72 hours), up to 96 hours, from 4 to 7 days, more than 7 days, from 7 to 30 days, or more than 30 days at 37 degrees Celsius (° C.). In some embodiments, a population of particles can have an average (e.g., outer) diameter of greater than 100 nanometers to up to 200 nanometers, for example, for 1 day (e.g., up to 24 hours), 2 days (e.g., up to 48 hours), 3 days (e.g., up to 72 hours), up to 96 hours, from 4 to 7 days, more than 7 days, from 7 to 30 days, or more than 30 days at 37 degrees Celsius (° C.). In some embodiments, a population of particles can have an average (e.g., outer) diameter of greater than 200 nanometers to up to 300 nanometers, for example, for 1 day (e.g., up to 24 hours), 2 days (e.g., up to 48 hours), 3 days (e.g., up to 72 hours), up to 96 hours, from 4 to 7 days, more than 7 days, from 7 to 30 days, or more than 30 days at 37 degrees Celsius (° C.). In some embodiments, a population of particles can have an average (e.g., outer) diameter of 1 nanometer to 20 nanometers, 1 nanometer to 30 nanometers, 1 nanometer to 50 nanometers, 1 nanometer to 75 nanometers, 1 nanometer to 100 nanometers, 1 nanometer to 125 nanometers, 1 nanometer to 150 nanometers, 1 nanometer to 175 nanometers, 1 nanometer to 200 nanometers, 1 nanometer to 300 nanometers, 1 nanometer to 1,000 nanometers, 20 nanometers to 30 nanometers, 20 nanometers to 50 nanometers, 20 nanometers to 75 nanometers, 20 nanometers to 100 nanometers, 20 nanometers to 125 nanometers, 20 nanometers to 150 nanometers, 20 nanometers to 175 nanometers, 20 nanometers to 200 nanometers, 20 nanometers to 300 nanometers, 20 nanometers to 1,000 nanometers, 30 nanometers to 50 nanometers, 30 nanometers to 75 nanometers, 30 nanometers to 100 nanometers, 30 nanometers to 125 nanometers, 30 nanometers to 150 nanometers, 30 nanometers to 175 nanometers, 30 nanometers to 200 nanometers, 30 nanometers to 300 nanometers, 30 nanometers to 1,000 nanometers, 50 nanometers to 75 nanometers, 50 nanometers to 100 nanometers, 50 nanometers to 125 nanometers, 50 nanometers to 150 nanometers, 50 nanometers to 175 nanometers, 50 nanometers to 200 nanometers, 50 nanometers to 300 nanometers, 50 nanometers to 1,000 nanometers, 75 nanometers to 100 nanometers, 75 nanometers to 125 nanometers, 75 nanometers to 150 nanometers, 75 nanometers to 175 nanometers, 75 nanometers to 200 nanometers, 75 nanometers to 300 nanometers, 75 nanometers to 1,000 nanometers, 100 nanometers to 125 nanometers, 100 nanometers to 150 nanometers, 100 nanometers to 175 nanometers, 100 nanometers to 200 nanometers, 100 nanometers to 300 nanometers, 100 nanometers to 1,000 nanometers, 125 nanometers to 150 nanometers, 125 nanometers to 175 nanometers, 125 nanometers to 200 nanometers, 125 nanometers to 300 nanometers, 125 nanometers to 1,000 nanometers, 150 nanometers to 175 nanometers, 150 nanometers to 200 nanometers, 150 nanometers to 300 nanometers, 150 nanometers to 1,000 nanometers, 175 nanometers to 200 nanometers, 175 nanometers to 300 nanometers, 175 nanometers to 1,000 nanometers, 200 nanometers to 300 nanometers, 200 nanometers to 1,000 nanometers, or 300 nanometers to 1,000 nanometers, for example, up to 30 days at 37 degrees Celsius (° C.). In some embodiments, a population of particles can have an average (e.g., outer) diameter of 1 nanometer, 20 nanometers, 30 nanometers, 50 nanometers, 75 nanometers, 100 nanometers, 125 nanometers, 150 nanometers, 175 nanometers, 200 nanometers, 300 nanometers, or 1,000 nanometers, for example, up to 30 days at 37 degrees Celsius (° C.). In some embodiments, a population of particles can have an average (e.g., outer) diameter of at least 1 nanometer, 20 nanometers, 30 nanometers, 50 nanometers, 75 nanometers, 100 nanometers, 125 nanometers, 150 nanometers, 175 nanometers, 200 nanometers, 300 nanometers, or 1,000 nanometers, for example, up to 30 days at 37 degrees Celsius (° C.). In some embodiments, a population of particles can have an average (e.g., outer) diameter of at most 1 nanometer, 20 nanometers, 30 nanometers, 50 nanometers, 75 nanometers, 100 nanometers, 125 nanometers, 150 nanometers, 175 nanometers, 200 nanometers, 300 nanometers, or 1,000 nanometers, for example, up to 30 days at 37 degrees Celsius (° C.). In some cases, an average diameter can be a mean hydrodynamic particle diameter.

A modulator and/or an antigen of the present disclosure may be associated with the particles of the present disclosure. For instance, in some embodiments, the antigens and STING modulators of the present disclosure can be positioned on different regions of the particles of the present disclosure. In some embodiments, a modulator (e.g., STING modulator) of the present disclosure can be encapsulated in a particle (e.g., STING modulators 16 encapsulated in particle 12, as illustrated in FIG. 1). In some embodiments, all or a portion of an antigen of the present disclosure can be associated with (e.g., attached to, adhered to, adsorbed onto, electrostatically interacted with, covalently bound to, noncovalently bound to, integrated into, or formulated onto) a surface of the particle (e.g., antigens 14 on surface of particle 12, as illustrated in FIG. 1). In some embodiments, all or a portion of an antigen is encapsulated within the lipid-based particle. In some embodiments, the association (e.g., adsorption) of an antigen with a surface of a particle can increase stability of the particle and/or increase delivery efficiency (e.g., to a target tissue) after administration.

In some embodiments, one or more antigens of the present disclosure can be encapsulated within a (e.g., lipid-based) particle of the present disclosure. In some embodiments, one or more modulators (e.g., including a STING modulator) of the present disclosure can be associated with an outer surface of the particle. In some embodiments, one or more antigens of the present disclosure can be encapsulated in a (e.g., lipid-based) particle while one or more modulator(s) (e.g., including a STING modulator) of the present disclosure are associated with an outer surface of the particle. In some embodiments, one or more antigens of the present disclosure can be associated with an outer surface of a (e.g., lipid-based) particle of this disclosure. In some embodiments, one or more modulators (e.g., including a STING modulator) of the present disclosure can be encapsulated within the particle. In some embodiments, one or more antigens of the present disclosure can be associated with an outer surface of a (e.g., lipid-based) particle of this disclosure while one or more modulators (e.g., including a STING modulator) of the present disclosure are encapsulated within the particle. In some embodiments, one or more antigens and one or more modulators (e.g., including a STING modulator) of the present disclosure can both be encapsulated within a (e.g., lipid-based) particle. In some embodiments, one or more antigens and one or more modulators (e.g., including a STING modulator) of the present disclosure may both be associated with a surface of a (e.g., lipid-based) particle of the present disclosure. In some embodiments, one or more antigens of the present disclosure can be integrated into a membrane of a (e.g., lipid-based) particle of the present disclosure. In some embodiments, one or more modulators (e.g., including a STING modulators) of the present disclosure can be integrated into a membrane of a (e.g., lipid-based) particle of the present disclosure. In some embodiments, one or more antigens of the present disclosure can be encapsulated within a (e.g., lipid-based) particle of the present disclosure while one or more modulators (e.g., including a STING modulator) are integrated into a membrane of a (e.g., lipid-based) particle of the present disclosure. In some embodiments, one or more antigens of the present disclosure can be associated with an outer surface of a (lipid-based) particle while one or more modulators (e.g., including a STING modulator) are integrated into a membrane of a (e.g., lipid-based) particle of the present disclosure. In some embodiments, one or more modulators (e.g., including a STING modulator) can be encapsulated within a (e.g., lipid-based) particle of the present disclosure while one or more antigens of the present disclosure are integrated into a membrane of the particle. In some embodiments, one or more modulators (e.g., including a STING modulator) can be associated with an outer surface of a (e.g., lipid-based) particle of the present disclosure while one or more antigens of the present disclosure are integrated into a membrane of the particle. In some embodiments, the incorporation of both an antigen and a modulator (e.g., a STING modulator) of the present disclosure within a (e.g., lipid-based) particle can facilitate coordinated cytosolic delivery.

In some embodiments, a composition described herein can comprise a divalent ion, such as a divalent cation. In some embodiments, a composition comprises a divalent cation encapsulated within, adsorbed onto, covalently coupled to, electrostatically interacted with, formulated onto a membrane (e.g., a membrane surface) of a particle (e.g., a lipid-based nanoparticle) described herein. In some embodiments, a composition can comprise a divalent cation selected from Mn²⁺, Mg²⁺, Ca²⁺, and Zn²⁺. For example, a composition can comprise a modulator (e.g., a STING agonist), a particle (e.g., a lipid-based nanoparticle), and a divalent cation, such as Mn²⁺, Mg²⁺, Ca²⁺, or Zn²⁺.

### Antigens

A composition useful in preventing or treating a respiratory disease or pathological condition in a subject can comprise an antigen. An antigen (or a portion thereof) of the present disclosure can be associated with (e.g., attached to, adhered to, adsorbed onto, electrostatically interacted with, covalently bound to, noncovalently bound to, integrated into, or formulated onto) a surface of the particle (e.g., a lipid-based particle, such as a liposome). In some embodiments, the antigen can be suitable for developing immunity in the subject against a disease. For instance, in some embodiments, one or more antigens in a composition of the present disclosure can be capable of eliciting an immune response in a subject against a disease.

An antigen of a composition described herein can include an attenuated or killed version of a pathogen, or portion thereof, that causes a disease (e.g., one or more of the pathogens described herein). In some embodiments, the antigen can comprise a peptide or a protein, or portion thereof, associated with the pathogen. In some embodiments, the antigen can comprise a surface protein (e.g., receptor protein), or portion thereof, of a pathogen. In some embodiments, the antigen may be in the form of a polynucleotide, for example, which may be used to express a second antigen (e.g., a plasmid DNA molecule expressing a second antigen).

An antigen of a composition described herein can comprise a spike protein or a portion thereof. In some embodiments, an antigen of a composition described herein can comprise at least one component of a coronavirus spike protein (S-protein). In some embodiments, an antigen of a composition described herein can comprise a monomeric form of a coronavirus spike protein (S-protein). In some embodiments, an antigen of a composition described herein can comprise a multimeric form of a coronavirus spike protein (S-protein).

In some embodiments, an antigen of the present disclosure can comprise a monomeric form of the SARS-CoV2 spike protein (S), a monomeric form of the receptor binding domain (RBD) of the SARS-CoV2 spike protein (S), a multimeric form of the SARS-CoV2 spike protein (S), a multimeric form of the receptor binding domain (RBD) of the SARS-CoV2 spike protein (S), a dimeric form of the SARS-CoV2 spike protein (S), a dimeric form of the receptor binding domain (RBD) of the SARS-CoV2 spike protein (S), a trimeric form of the SARS-CoV2 spike protein (S), a trimeric form of the receptor binding domain (RBD) of the SARS-CoV2 spike protein (S), or combinations thereof. In some embodiments, an antigen of a composition can comprise a chimeric protein (e.g., a chimeric spike protein).

In some embodiments, an antigen can comprise a monomeric or multimeric form of the SARS-CoV2 spike protein (S) containing the D614G mutation, A222V mutation, S477N mutation, D80Y mutation, S98F mutation, or a combination thereof.

In some embodiments, the antigen can be a mixture of SARS-CoV2 spike proteins harboring different mutations. In some embodiments, the antigen can comprise the monomeric or multimeric form of a nucleocapsid protein. For example, an antigen of the present disclosure can comprise a monomeric or multimeric form of the SARS-CoV2 nucleocapsid (N) protein. In some embodiments, the antigen can be the monomeric or multimeric form of the SARS-CoV2 nucleocapsid (N) protein containing the A220V mutation.

In some embodiments, an antigen can comprise an influenza virus antigen or portion thereof, an influenza A virus antigen or portion thereof, an influenza B virus antigen or portion thereof, a parainfluenza virus antigen or portion thereof, an adenovirus antigen or portion thereof, an enterovirus antigen or portion thereof, a coronavirus antigen or portion thereof, a respiratory syncytial virus (RSV) antigen or portion thereof, a rhinovirus antigen or portion thereof, a DNA virus antigen or portion thereof, an RNA virus antigen or portion thereof, or a combination thereof. In some embodiments, an antigen can comprise an antigen of a severe acute respiratory syndrome coronavirus (SARS-CoV), a severe acute respiratory syndrome-related coronavirus (SARSr-CoV), a human coronavirus 229E (HCoV-229E), a human coronavirus NL63 (HCoV-NL63), a human coronavirus 0C43 (HCoV-0C43), a human coronavirus HKU1 (HCoV-HKU1), a Middle East respiratory syndrome-related coronavirus (MERS-CoV), a severe acute respiratory syndrome-related coronavirus 2 (SARS-CoV-2), a variant of SARS-CoV-2 (e.g., 20A.EU1, or spike variant D614G), or a combination thereof. In some embodiments, an antigen can comprise an (e.g., coronavirus) alpha variant spike protein monomer, an (e.g., coronavirus) alpha variant spike protein trimer, a (e.g., coronavirus) beta variant spike protein monomer, a (e.g., coronavirus) beta variant spike protein trimer, a (e.g., coronavirus) gamma variant spike protein monomer, a (e.g., coronavirus) gamma variant spike protein trimer, a (e.g., coronavirus) delta variant spike protein monomer, a (e.g., coronavirus) delta variant spike protein trimer, a receptor binding domain (RBD) portion of an (e.g., coronavirus) alpha variant spike protein, an RBD portion of a (e.g., coronavirus) beta variant spike protein, an RBD portion of a (e.g., coronavirus) gamma variant spike protein, and/or an RBD portion of a (e.g., coronavirus) delta variant spike protein.

In some embodiments, an antigen (e.g., an antigen comprising a nucleocapsid (N) protein) of a composition or method described herein can have an amino acid sequence of according to SEQ ID NO: 1. In some embodiments, an antigen (e.g., an antigen comprising a nucleocapsid protein) of a composition or method described herein can have an amino acid sequence that is at least 70%, at least 75%, at least 80%, at least 85%, at least 90%, at least 95%, at least 97%, at least 99%, or 100% identical to SEQ ID NO: 1. In some embodiments, an antigen (e.g., an antigen comprising a nucleocapsid protein) can be encoded by a nucleotide sequence according to SEQ ID NO: 2. In some embodiments, an antigen can be encoded by a nucleotide sequence that is at least 80%, at least 85%, at least 90%, at least 95%, at least 97%, at least 98%, at least 99%, greater than 99%, or 100% sequence identical to SEQ ID NO: 2. In some embodiments, an antigen (e.g., an antigen comprising a spike (S) protein) of a composition or method described herein can have an amino acid sequence of SEQ ID NO: 3. In some embodiments, an antigen (e.g., an antigen comprising a spike (S) protein) of a composition or method described herein can have an amino acid sequence with at least 70%, at least 75%, at least 80%, at least 85%, at least 90%, at least 95%, at least 97%, at least 99%, or 100% identity with SEQ ID NO: 3. In some embodiments, an antigen (e.g., an antigen comprising a spike (S) protein) can be encoded by a nucleotide sequence according to SEQ ID NO: 4. In some embodiments, an antigen (e.g., an antigen comprising a spike (S) protein) can be encoded by a nucleotide sequence having at least 80%, at least 85%, at least 90%, at least 95%, at least 97%, at least 98%, at least 99%, greater than 99%, or 100% sequence identity with SEQ ID NO: 4.

In some embodiments, it can be advantageous for a composition described herein (e.g., wherein the composition is utilized in a method described herein) to comprise both an N-protein and an S-protein. For example, a composition described herein can comprise an N-protein having an amino acid sequence that is at least 90% identical to SEQ ID NO: 1 and an S-protein having an amino acid sequence that is at least 75%, at least 80%, at least 85%, at least 90%, at least 95%, at least 99%, or 100% identical to SEQ ID NO: 3. For example, a composition described herein can comprise an N-protein having an amino acid sequence that is at least 95% identical to SEQ ID NO: 1 and an S-protein having an amino acid sequence that is at least 75%, at least 80%, at least 85%, at least 90%, at least 95%, at least 99%, or 100% identical to SEQ ID NO: 3. For example, a composition described herein can comprise an N-protein having an amino acid sequence that is 100% identical to SEQ ID NO: 1 and an S-protein having an amino acid sequence that is at least 75%, at least 80%, at least 85%, at least 90%, at least 95%, at least 99%, or 100% identical to SEQ ID NO: 3.

In some embodiments, it can be beneficial for a composition described herein to comprise equal amounts of N-protein and S-protein. In some embodiments, a composition or method described herein can benefit from adjusting the ratio of N-proteins to S-proteins such that the composition comprises unequal amounts of N-protein and S-protein. For example, a composition described herein can comprise more N-protein than S-protein. In some embodiments, it can be beneficial for a composition described herein to comprise more S-protein than N-protein. In some embodiments, a composition described herein can comprise a mass ratio of N-protein to S-protein (e.g., mass ratio of N-protein:S-protein) of less than 1:1,000, from 1:1,000 to 1:500, from 1:500 to 1:200, from 1:200 to 1:100, from 1:100 to 1:50, from 1:50 to 1:25, from 1:25 to 1:20, from 1:20 to 1:10, from 1:10 to 1:5, from 1:5 to 1:4, from 1:4 to 1:3, from 1:3 to 1:2, from 1:2 to 2:3, from 2:3 to 3:4, from 3:4 to 4:5, from 4:5 to 1:1, from 1:1 to 5:4, from 5:4 to 4:3, from 4:3 to 3:2, from 3:2 to 2:1, from 2:1 to 3:1, from 3:1 to 4:1, from 4:1 to 5:1, from 5:1 to 10:1, from 10:1 to 20:1, from 20:1 to 25:1, from 25:1 to 50:1, from 50:1 to 100:1, from 100:1 to 200:1, from 200:1 to 500:1, from 500:1 to 1,000:1, or greater than 1,000:1. In some embodiments, the mass ratio of a spike protein of a composition described herein to a nucleocapsid protein of the composition is 1:2. In some embodiments, a composition described herein can comprise a molar ratio of N-protein to S-protein (e.g., molar ratio of N-protein:S-protein) of less than 1:1,000, from 1:1,000 to 1:500, from 1:500 to 1:200, from 1:200 to 1:100, from 1:100 to 1:50, from 1:50 to 1:25, from 1:25 to 1:20, from 1:20 to 1:10, from 1:10 to 1:5, from 1:5 to 1:4, from 1:4 to 1:3, from 1:3 to 1:2, from 1:2 to 2:3, from 2:3 to 3:4, from 3:4 to 4:5, from 4:5 to 1:1, from 1:1 to 5:4, from 5:4 to 4:3, from 4:3 to 3:2, from 3:2 to 2:1, from 2:1 to 3:1, from 3:1 to 4:1, from 4:1 to 5:1, from 5:1 to 10:1, from 10:1 to 20:1, from 20:1 to 25:1, from 25:1 to 50:1, from 50:1 to 100:1, from 100:1 to 200:1, from 200:1 to 500:1, from 500:1 to 1,000:1, or greater than 1,000:1.

In some embodiments, a composition or method described herein can comprise an N-protein that is associated with (e.g., embedded in, covalently coupled to, or non-covalently coupled to) a membrane of a (e.g., lipid-based) nanoparticle and an S-protein that is not associated with a membrane of the nanoparticle (e.g., contained within the nanoparticle or outside of and uncoupled to the nanoparticle (e.g., in co-treatment)). In some embodiments, a composition or method described herein can comprise an S-protein that is associated with (e.g., embedded in, covalently coupled to, or non-covalently coupled to) a membrane of a (e.g., lipid-based) nanoparticle and an N-protein that is not associated with a membrane of the nanoparticle (e.g., contained within the nanoparticle or outside of and uncoupled to the nanoparticle (e.g., in co-treatment)). In some embodiments, a composition or method described herein can comprise an N-protein that is associated (e.g., embedded in, covalently coupled to, or non-covalently coupled to) with the membrane of a (e.g., lipid-based) nanoparticle and an S-protein that is associated with the membrane of the nanoparticle. In some embodiments, a composition or method described herein can comprise an N-protein that is unassociated (e.g., contained within the nanoparticle or outside of and uncoupled to the nanoparticle (e.g., in co-treatment)) with the membrane of a (e.g., lipid-based) nanoparticle and an S-protein that is unassociated with the membrane of the nanoparticle.

In some embodiments, the antigen can be a mixture of SARS-CoV2 spike protein and nucleocapsid protein. In some embodiments, the antigen can be a mixture of SARS-CoV2 spike proteins harboring different mutations and a nucleocapsid protein.

In some embodiments, an antigen can be an influenza virus antigen or portion thereof, an influenza A virus antigen or portion thereof, an influenza B virus antigen or portion thereof, a parainfluenza virus antigen or portion thereof, an adenovirus antigen or portion thereof, an enterovirus antigen or portion thereof, a coronavirus antigen or portion thereof, a respiratory syncytial virus (RSV) antigen or portion thereof, a rhinovirus antigen or portion thereof, a DNA virus antigen or portion thereof, an RNA virus antigen or portion thereof, or a combination thereof.

In some embodiments, a composition of the present disclosure is suitable for developing immunity in the subject against a cancer. In some embodiments, an antigen of a composition of the present disclosure is suitable for developing immunity in the subject against a cancer. For instance, in some embodiments, the antigens in the therapeutic compositions of the present disclosure are capable of eliciting an immune response in a subject against a cancer (e.g., cancers described previously).

In some embodiments, an antigen of a composition of the present disclosure can be an attenuated or killed version of a tumor cell (or portion thereof) associated with a cancer. In some embodiments, the antigen can comprise a peptide or a protein associated with a cancer. In some embodiments, the antigen can comprise a surface protein (e.g., a receptor protein) of a cancer cell.

In some embodiments, the antigen can comprise a mutated protein of a cancer cell. In some embodiments, the antigen can be a synthetic long peptide targeting cancer mutations.

An antigen of a composition of the present disclosure can be in various forms. For instance, in some embodiments, the antigens can be recombinant peptides or proteins, or peptide epitopes recognized by T-cells. In some embodiments, antigens of a composition disclosed herein can comprise tandem minigenes. In some embodiments, the antigen can be in the form of a nucleotide expressing the antigen (e.g., a plasmid DNA molecule expressing the antigen).

### Modulators

A composition described herein for preventing or treating a disease in a subject (e.g., comprising a particle, such as a lipid-based particle, and, optionally, an antigen) can comprise a modulator. A modulator of a composition described herein can be a pattern recognition receptor modulator, such as a STING modulator (e.g., STING pathway modulator). In some embodiments, a modulator of a composition described herein can be a pattern recognition receptor agonist. In some embodiments, a modulator of a composition described herein can be a pattern recognition receptor antagonist. A composition of the present disclosure may include various types of STING modulators. For instance, in some embodiments, the STING modulator is an antagonist of the STING pathway. In some embodiments, the STING modulator is an agonist of the STING pathway. In some embodiments, a modulator of a composition described herein is capable of activating the STING pathway in a subject (e.g., to whom the composition is administered). In some embodiments, a modulator of a composition described herein is capable of inhibiting the STING pathway in a subject (e.g., to whom the composition is administered).

A STING modulator of a composition described herein can comprise bis-(3′,5′)-cyclic dimeric guanosine monophosphate (c-di-GMP), cyclic guanosine monophosphate-adenosine monophosphate (cGAMP), amidobenzimidazole, derivatives of amidobenzimidazole, nucleotide modulators, plasmid DNA modulators, nucleic acid modulators, CF501, SHR1032, or a combination thereof.

In some embodiments, the STING modulator can be an antagonist of the STING pathway. In some embodiments, a STING antagonist may be especially effective in treating and/or prevention of diseases, e.g., where a pathogen causing the disease (e.g., RNA viruses such as rhinoviruses) utilizes the STING pathway to promote viral replication. In some embodiments, a STING antagonist can be utilized to inhibit viral replication by reducing viral access to the STING pathway. In some embodiments, the STING antagonists include, without limitation, C-178, H-151, and combinations thereof.

In some embodiments, a modulator of a composition described herein can be disposed within an interior space of a particle of the composition. For instance, a modulator can be encapsulated within a particle (e.g., a lipid-based particle, such as a liposome) of a composition described herein. In some embodiments, a modulator of a composition described herein can be associated with a surface (e.g., an outer surface of a membrane) of a particle of the composition. In some embodiments, a modulator associated with a surface of a particle is associated by being covalently or non-covalently bound to the surface of the particle. In some embodiments, a modulator associated with the surface of the particle is associated by being integrated into the surface (e.g., membrane) of the particle.

In some embodiments, a nucleic acid sequence of an antigen of a composition described herein and a nucleic acid sequence of a modulator of the composition can be at least 85%, at least 90%, at least 95%, or 100% identical. In some embodiments, a portion of a nucleic acid sequence of an antigen of a composition described herein can be at least 85%, at least 90%, at least 95%, or 100% identical to a nucleic acid sequence of a modulator of the composition. In some embodiments, an antigen comprising a nucleic acid sequence that is at least 85%, at least 90%, at least 95%, or 100% identical to a nucleic acid sequence of a modulator described herein can be encapsulated within a particle (e.g., a lipid-based particle) of a composition described herein (e.g., wherein the modulator is also encapsulated within the lipid-based particle). In some embodiments, an antigen and a modulator described herein (e.g., wherein the antigen and the modulator comprise nucleic acid sequences that is at least 85%, at least 90%, at least 95%, or 100% identical) are both encapsulated within a lipid-based particle described herein. In some embodiments, a composition comprising an antigen and modulator comprising sequences that are at least 85%, at least 90%, at least 95%, or 100% identical can be delivered intranasally.

In some cases, it can be advantageous to formulate compositions described herein within a desired pH range. For example, formulation of a composition described herein (e.g., for intranasal delivery) between pH 4.0 and pH 7.5 (e.g., pH 4.5 to pH 6.5 or pH 5.5 to pH 6.5) can avoid irritation and/or histological damage to the intranasal tissue, which may occur if extremely acidic or basic formulations are used for intranasal delivery (e.g., because nasal cavity pH can be from about 5.5 to 6.5). In some cases, a composition described herein can have a pH of 4.0 to 7.5. In some cases, a composition described herein can have a pH of 4.0 to 4.5, 4.0 to 4.8, 4.0 to 5.0, 4.0 to 5.2, 4.0 to 5.5, 4.0 to 5.8, 4.0 to 6, 4.0 to 6.2, 4.0 to 6.5, 4.0 to 7, 4.0 to 7.5, 4.5 to 4.8, 4.5 to 5.0, 4.5 to 5.2, 4.5 to 5.5, 4.5 to 5.8, 4.5 to 6.0, 4.5 to 6.2, 4.5 to 6.5, 4.5 to 7.0, 4.5 to 7.5, 4.8 to 5.0, 4.8 to 5.2, 4.8 to 5.5, 4.8 to 5.8, 4.8 to 6.0, 4.8 to 6.2, 4.8 to 6.5, 4.8 to 7.0, 4.8 to 7.5, 5.0 to 5.2, 5.0 to 5.5, 5.0 to 5.8, 5.0 to 6.0, 5.0 to 6.2, 5.0 to 6.5, 5.0 to 7.0, 5 to 7.5, 5.2 to 5.5, 5.2 to 5.8, 5.2 to 6.0, 5.2 to 6.2, 5.2 to 6.5, 5.2 to 7.0, 5.2 to 7.5, 5.5 to 5.8, 5.5 to 6.0, 5.5 to 6.2, 5.5 to 6.5, 5.5 to 7.0, 5.5 to 7.5, 5.8 to 6.0, 5.8 to 6.2, 5.8 to 6.5, 5.8 to 7.0, 5.8 to 7.5, 6.0 to 6.2, 6.0 to 6.5, 6.0 to 7, 6.0 to 7.5, 6.2 to 6.5, 6.2 to 7, 6.2 to 7.5, 6.5 to 7.0, 6.5 to 7.5, or 7.0 to 7.5. In some cases, a composition described herein can have a pH of 4.0, 4.5, 4.8, 5.0, 5.2, 5.5, 5.8, 6.0, 6.2, 6.5, 7.0, or 7.5. In some cases, a composition described herein can have a pH of at least 4.0, 4.5, 4.8, 5.0, 5.2, 5.5, 5.8, 6.0, 6.2, 6.5, 7.0, or 7.5. In some cases, a composition described herein can have a pH of at most 4.0, 4.5, 4.8, 5.0, 5.2, 5.5, 5.8, 6.0, 6.2, 6.5, 7.0, or 7.5.

In some cases, it can be advantageous to formulate compositions described herein within a desired osmolarity range. For example, formulation of a composition described herein (e.g., for intranasal delivery) having an osmolarity from 50 to 900 mOsm/kg can improve absorption while avoiding potential epithelial damage (e.g., which may occur from compositions with very low osmolarities) and avoiding increases to mucosal secretions (e.g., which may occur from compositions with very high osmolarities) (e.g., because nasal cavity osmolarity can be about 280 mOsm/kg). In some cases, a composition described herein can have an osmolarity of 50 mOsm/kg to 900 mOsm/kg. In some cases, a composition described herein can have an osmolarity of 50 mOsm/kg to 150 mOsm/kg, 50 mOsm/kg to 250 mOsm/kg, 50 mOsm/kg to 300 mOsm/kg, 50 mOsm/kg to 350 mOsm/kg, 50 mOsm/kg to 450 mOsm/kg, 50 mOsm/kg to 550 mOsm/kg, 50 mOsm/kg to 650 mOsm/kg, 50 mOsm/kg to 750 mOsm/kg, 50 mOsm/kg to 850 mOsm/kg, 50 mOsm/kg to 900 mOsm/kg, 150 mOsm/kg to 250 mOsm/kg, 150 mOsm/kg to 300 mOsm/kg, 150 mOsm/kg to 350 mOsm/kg, 150 mOsm/kg to 450 mOsm/kg, 150 mOsm/kg to 550 mOsm/kg, 150 mOsm/kg to 650 mOsm/kg, 150 mOsm/kg to 750 mOsm/kg, 150 mOsm/kg to 850 mOsm/kg, 150 mOsm/kg to 900 mOsm/kg, 250 mOsm/kg to 300 mOsm/kg, 250 mOsm/kg to 350 mOsm/kg, 250 mOsm/kg to 450 mOsm/kg, 250 mOsm/kg to 550 mOsm/kg, 250 mOsm/kg to 650 mOsm/kg, 250 mOsm/kg to 750 mOsm/kg, 250 mOsm/kg to 850 mOsm/kg, 250 mOsm/kg to 900 mOsm/kg, 300 mOsm/kg to 350 mOsm/kg, 300 mOsm/kg to 450 mOsm/kg, 300 mOsm/kg to 550 mOsm/kg, 300 mOsm/kg to 650 mOsm/kg, 300 mOsm/kg to 750 mOsm/kg, 300 mOsm/kg to 850 mOsm/kg, 300 mOsm/kg to 900 mOsm/kg, 350 mOsm/kg to 450 mOsm/kg, 350 mOsm/kg to 550 mOsm/kg, 350 mOsm/kg to 650 mOsm/kg, 350 mOsm/kg to 750 mOsm/kg, 350 mOsm/kg to 850 mOsm/kg, 350 mOsm/kg to 900 mOsm/kg, 450 mOsm/kg to 550 mOsm/kg, 450 mOsm/kg to 650 mOsm/kg, 450 mOsm/kg to 750 mOsm/kg, 450 mOsm/kg to 850 mOsm/kg, 450 mOsm/kg to 900 mOsm/kg, 550 mOsm/kg to 650 mOsm/kg, 550 mOsm/kg to 750 mOsm/kg, 550 mOsm/kg to 850 mOsm/kg, 550 mOsm/kg to 900 mOsm/kg, 650 mOsm/kg to 750 mOsm/kg, 650 mOsm/kg to 850 mOsm/kg, 650 mOsm/kg to 900 mOsm/kg, 750 mOsm/kg to 850 mOsm/kg, 750 mOsm/kg to 900 mOsm/kg, or 850 mOsm/kg to 900 mOsm/kg. In some cases, a composition described herein can have an osmolarity of 50 mOsm/kg, 150 mOsm/kg, 250 mOsm/kg, 300 mOsm/kg, 350 mOsm/kg, 450 mOsm/kg, 550 mOsm/kg, 650 mOsm/kg, 750 mOsm/kg, 850 mOsm/kg, or 900 mOsm/kg. In some cases, a composition described herein can have an osmolarity of at least 50 mOsm/kg, 150 mOsm/kg, 250 mOsm/kg, 300 mOsm/kg, 350 mOsm/kg, 450 mOsm/kg, 550 mOsm/kg, 650 mOsm/kg, 750 mOsm/kg, 850 mOsm/kg, or 900 mOsm/kg. In some cases, a composition described herein can have an osmolarity of at most 50 mOsm/kg, 150 mOsm/kg, 250 mOsm/kg, 300 mOsm/kg, 350 mOsm/kg, 450 mOsm/kg, 550 mOsm/kg, 650 mOsm/kg, 750 mOsm/kg, 850 mOsm/kg, or 900 mOsm/kg.

In some cases, it can be advantageous to formulate compositions described herein within a desired viscosity range. For example, formulation of a composition described herein (e.g., for intranasal delivery) having a viscosity, for example, from 1.1 cP (centipoise) to 50 cP (e.g., 1.5 cP to 50 cP) can increase residence time in the nasal cavity while adversely affecting droplet size (e.g., which can affect spray pattern and/or distribution within the nasal cavity). In some cases, a composition described herein can have a viscosity of 1 cP (centipoise) to 100 cP. In some cases, a composition described herein can have a viscosity of 1 cP to 1.1 cP, 1 cP to 1.5 cP, 1 cP to 2 cP, 1 cP to 5 cP, 1 cP to 7 cP, 1 cP to 10 cP, 1 cP to 15 cP, 1 cP to 25 cP, 1 cP to 50 cP, 1 cP to 100 cP, 1.1 cP to 1.5 cP, 1.1 cP to 2 cP, 1.1 cP to 5 cP, 1.1 cP to 7 cP, 1.1 cP to 10 cP, 1.1 cP to 15 cP, 1.1 cP to 25 cP, 1.1 cP to 50 cP, 1.1 cP to 100 cP, 1.5 cP to 2 cP, 1.5 cP to 5 cP, 1.5 cP to 7 cP, 1.5 cP to 10 cP, 1.5 cP to 15 cP, 1.5 cP to 25 cP, 1.5 cP to 50 cP, 1.5 cP to 100 cP, 2 cP to 5 cP, 2 cP to 7 cP, 2 cP to 10 cP, 2 cP to 15 cP, 2 cP to 25 cP, 2 cP to 50 cP, 2 cP to 100 cP, 5 cP to 7 cP, 5 cP to 10 cP, 5 cP to 15 cP, 5 cP to 25 cP, 5 cP to 50 cP, 5 cP to 100 cP, 7 cP to 10 cP, 7 cP to 15 cP, 7 cP to 25 cP, 7 cP to 50 cP, 7 cP to 100 cP, 10 cP to 15 cP, 10 cP to 25 cP, 10 cP to 50 cP, 10 cP to 100 cP, 15 cP to 25 cP, 15 cP to 50 cP, 15 cP to 100 cP, 25 cP to 50 cP, 25 cP to 100 cP, or 50 cP to 100 cP. In some cases, a composition described herein can have a viscosity of 1 cP, 1.1 cP, 1.5 cP, 2 cP, 5 cP, 7 cP, 10 cP, 15 cP, 25 cP, 50 cP, or 100 cP. In some cases, a composition described herein can have a viscosity of at least 1 cP, 1.1 cP, 1.5 cP, 2 cP, 5 cP, 7 cP, 10 cP, 15 cP, 25 cP, 50 cP, or 100 cP. In some cases, a composition described herein can have a viscosity of at most 1.0 cP, 1.1 cP, 1.5 cP, 2 cP, 5 cP, 7 cP, 10 cP, 15 cP, 25 cP, 50 cP, or 100 cP.

### Methods of Use

In additional embodiments, the present disclosure pertains to methods of treating or preventing a disease in a subject by administering the therapeutic compositions of the present disclosure to the subject. In more specific embodiments illustrated in FIG. 2A, the methods of the present disclosure include a step of administering a therapeutic composition to the subject (step 20) in order to treat or prevent a disease in the subject (step 22).

As set forth in more detail herein, the therapeutic compositions and methods of the present disclosure can have numerous embodiments. For instance, the therapeutic compositions of the present disclosure can include various types of STING modulators and antigens. Moreover, the methods of the present disclosure can be utilized to administer the therapeutic compositions of the present disclosure to numerous subjects in order to treat or prevent various diseases in various manners.

### Applications

In some embodiments, compositions and/or methods described herein may be useful in preventing or treating a respiratory disease or condition, such as a respiratory disease or condition related to influenza, coronavirus, respiratory syncytial viruses, rhinoviruses, or combinations thereof. In some embodiments, compositions and/or methods described herein can be useful in preventing or treating severe acute respiratory syndrome (SARS), acute respiratory distress syndrome (ARDS), and/or hypercytokinemia (e.g., cytokine storm). In some embodiments, compositions and/or methods described herein can be useful in preventing and/or treating a cancer, such as a lung cancer.

There is an urgent need for a safe and efficacious vaccine against respiratory diseases and cancer. In some embodiments, a composition described herein can comprise a vaccine or therapeutic treatment (e.g., delivered intranasally) containing a liposome containing agonists of the stimulator of interferon genes (STING) pathway to enable humoral immunity, T-cell immunity, systemic immunity and/or mucosal immunity.

Innate immunity is the first line of defense against invading pathogens. Innate immunity (unlike adaptive immunity, which can be customized for each pathogen) can be triggered by pattern recognition receptors (PRR) on the host cells that recognize conserved pathogen-associated molecular patterns (PAMPs)1. This mode of recognition can ensure that there is an immediate response that does not need customization.

In the context of RNA viruses, the recognition and activation of virus-specific RNA molecules can lead to the activation of interferon regulator factors (IRFs) and nuclear factor κB (NF-κB). Together, these transcriptional regulators launch broad antiviral programs, including the synthesis and secretion of type-I and type-III interferons and the subsequent upregulation of IFN-stimulated genes (ISGs).

This comprehensive antiviral program can apply a strong selection pressure on viral replication. Viruses have evolved elaborate countermeasures to interfere with interferon signaling. The interplay between the interferon mediated response and the viral countermeasures is heterogeneous and may be an explanation for the heterogeneity in morbidity and mortality seen in humans.

The type-1 interferon response can be suppressed in pathogen infections (e.g., coronavirus or other respiratory pathogens) and the balance between the ISGs and a pro-inflammatory response mediated by NF-κB can subsequently be dysregulated. Consequently, patients with advanced disease upon pathogen infection (e.g., respiratory pathogen infection) may have low interferon signaling but exaggerated tumor necrosis factor (TNF) and interleukin-6 (IL-6) secretion. Indeed, humans with autoantibodies against interferons that neutralize interferon function can be at high risk of developing advanced disease.

Similarly, humans with inborn errors of type I IFN immunity may develop life-threatening COVID-19 at a higher rate than those without such errors. In some embodiments, pretreatment of cell lines with type 1 interferon can inhibit viral replication in otherwise susceptible cells. Without being bound by theory, these considerations may help to explain that a lack of a robust type I IFN response can underlie advanced COVID-19.

The stimulator of interferon genes (STING) pathway is a PRR that senses cyclic DNA dinucleotides and activates IRF3 and NF-κB leading to the synthesis of ISGs. Although primarily thought to be important for sensing bacteria and DNA viruses, the role of STING in RNA virus-mediated type I IFN and cytokine production needs more detailed study. As shown herein, the role of STING in RNA virus-mediated type I IFN and cytokine production may be virus and cell type specific.

The sensing of double stranded RNA derived from viruses like the coronavirus within the cytoplasm of human cells can be accomplished by the RIG-I like receptors, including the retinoic acid inducible gene 1 (RIG-1) and melanoma differentiation gene 5 (MDA5). In some embodiments, the sensing pathways downstream of these receptors lead to the activation of the two IKK-related kinases, TANK-binding kinase 1 (TBK1) and inducible IκB kinase (IKKi). In some embodiments, these kinases are also engaged by the activated STING pathway, suggesting that while the PRRs have evolved to sense different molecules they may converge in their downstream responses.

Utilizing STING agonists as therapeutics can take advantage of this conserved downstream effector responses to promote a balanced activation of type I interferons, even in the context of SARS-CoV2 infection. Enabling the release of type I interferons with STING agonists independent of the RIG-1 like receptors increases the likelihood that this is not subjected to countermeasures evolved by the virus.

The release of interferons may enable a broad antiviral program limiting viral replication and thus mitigate disease severity. On the other hand, RNA viruses like rhinoviruses can hijack the STING pathway to promote viral replication. In this context, it can be useful to inhibit viral replication by utilizing a STING antagonist to dampen inflammation.

Without being bound by theory, the therapeutic compositions of the present disclosure can be used to treat or prevent a disease through various mechanisms of action. For instance, in some embodiments, the therapeutic compositions of the present disclosure can be used to treat a disease in a subject. In some embodiments, the therapeutic compositions of the present disclosure can be used to prevent a disease in a subject. In some embodiments, the therapeutic compositions of the present disclosure can be used to treat and prevent a disease in a subject. In some embodiments, the therapeutic compositions of the present disclosure can be used to treat or prevent a disease in a subject by developing immunity in the subject against the disease.

For instance, in some embodiments, the therapeutic compositions of the present disclosure can elicit an immune response in the subject against the disease. In some embodiments, the therapeutic compositions of the present disclosure can elicit such immunity through at least one of innate immunity, mucosal immunity, systemic immunity, cellular immunity, humoral immunity, T-cell immunity, production of systemic neutralizing antibodies, induction of IgG responses, induction of IgA responses, induction of IgM responses, induction of T-cell responses, induction of mucosal IgA responses in lung and nasal compartments, induction of Th1 T-cell responses, induction of CD8+ T-cell responses, induction of CD4+ T cell responses, induction of NK cell responses, activation or inhibition of the stimulator of interferon genes (STING) pathway, or combinations thereof.

In some embodiments, a composition disclosed herein can elicit a mean dilution titer (e.g., of an IgG or an IgA specific to a pathogen antigen, or portion thereof) in the serum of a subject at least 1:25, greater than 1:25. In some embodiments, a composition disclosed herein can elicit a mean dilution titer (e.g., of an IgG or an IgA specific to a pathogen antigen, or portion thereof) in the bronchoalveolar lavage fluid (BALF) of a subject of from 1:25 to 1:50, from 1:30 to 1:50, from 1:40 to 1:50, from 1:50 to 1:60, or at least 1:25, greater than 1:25, or less than 1:60.

In some embodiments, compositions of the present disclosure can provoke or aid in the development of immunity against a disease in a subject, e.g., by eliciting an innate immune response in the subject against the disease. In some embodiments, the elicited innate immune response can lead to the activation of interferon regulator factors (IRFs), nuclear factor KB (NF-κB), or combinations thereof. In some embodiments, the elicited innate immune response can result in the synthesis and secretion of type-I and type-III interferons (IFNs) and the subsequent upregulation of IFN-stimulated genes (ISGs).

In some embodiments, the therapeutic compositions of the present disclosure can be used to develop immunity in a subject against a disease, e.g., through mucosal immunity and/or systemic immunity. In some embodiments, the therapeutic compositions of the present disclosure can aid in developing immunity in a subject through mucosal immunity, systemic immunity, and cellular immunity. In some embodiments, systemic immunity can be developed through the production of neutralizing antibodies against an antigen. In some embodiments, cellular immunity may be developed in spleen cells and/or lung cells. In some embodiments, mucosal immunity can be developed through production of IgA in the nasal compartment and lung, and IgA secreting cells in the spleen.

The methods and therapeutic compositions of the present disclosure can provide numerous advantages in various embodiments. For instance, in some embodiments, the therapeutic compositions of the present disclosure can be administered to large populations without the need for large clinical facilities.

Moreover, in some embodiments, the therapeutic compositions of the present disclosure can prevent establishment of an initial viral reservoir (e.g., in a nasal compartment) and may help to control viral dissemination between individuals and/or dissemination within an individual.

In some embodiments, the therapeutic compositions of the present disclosure can correct a deficiency in interferon activation, which may be one of the primary mechanisms of escape mediated by respiratory viruses. Additionally, the therapeutic compositions of the present disclosure can provide universal mucosal adjuvants for developing broad-spectrum therapeutic compositions against numerous pathogens, such as coronaviruses or other respiratory pathogens (including other viral respiratory pathogens), e.g., because the therapeutic compositions of the present disclosure can be utilized in some embodiments to elicit one or more of IgM, IgG, IgA and T-cell responses in subjects.

In some embodiments, the methods and therapeutic compositions of the present disclosure can have veterinary applications. In some embodiments, the veterinary applications include the treatment or prevention of diseases in different types of animals and other similar applications.

### Formulation and Administration

Compositions of the present disclosure can be in various forms. In some embodiments, a composition described herein can be delivered as a solubilized liquid. In some embodiments, the therapeutic compositions of the present disclosure are suitable for intranasal and/or inhalational administration to a subject. In some embodiments, intranasal delivery of compositions disclosed herein can be used to target intranasal compartment tissues. In some embodiments, inhalational administration of compositions disclosed herein can be used to target lung compartment tissues.

In some embodiments, a syringe or liquid dropper can be used to administer a composition described herein intranasally to a subject. In some embodiments, administering a composition described herein to a subject can comprise the use of a spray nozzle, a nebulizer, and/or an atomizer. In some embodiments, the therapeutic compositions of the present disclosure can be self-administered.

In some embodiments, a composition of the present disclosure also includes one or more stabilizers. In some embodiments, the stabilizers include, without limitation, anti-oxidants, sequestrants, ultraviolet stabilizers, or combinations thereof.

In some embodiments, a composition of the present disclosure also includes one or more surfactants. In some embodiments, the surfactants include, without limitation, anionic surfactants, sugars, cationic surfactants, zwitterionic surfactants, non-ionic surfactants, or combinations thereof.

In some embodiments, a composition of the present disclosure also includes one or more excipients. In some embodiments, the excipients include, without limitation, lactose, sucrose, starch powder, cellulose esters of alkanoic acids, trehalose, cellulose alkyl esters, talc, stearic acid, magnesium stearate, magnesium oxide, sodium and calcium salts of phosphoric and sulfuric acids, gelatin, acacia gum, trehalose, sodium alginate, polyvinylpyrrolidone, polyvinyl alcohol, or combinations thereof.

In some embodiments, a composition of the present disclosure can be lyophilized (e.g., freeze-dried). Lyophilization of a composition of the present disclosure (or a component thereof) can increase the storage stability (e.g., shelf stability) of a composition described herein. In some embodiments, one or more components of a composition of the present disclosure can be lyophilized. In some embodiments, lyophilization of a composition of the present disclosure (or a component thereof) can allow for easy preparation of the composition, e.g., allowing use (e.g., administration to a subject) in regions without easy access to material preparation facilities. For instance, a modulator, a lipid-based particle, and/or an antigen described herein can be lyophilized and then rehydrated and mixed (e.g., as described herein) to formulate a composition described herein at a site of administration to a subject that is remote from a permanent medical or pharmaceutical facility. In some embodiments, a lyophilized composition described herein (or a portion thereof, such as a lyophilized particle or a lyophilized antigen or a lyophilized modulator) can be stored at 4° C. In some embodiments, a lyophilized composition described herein (or a portion thereof) can be stored at 4° C. and used for up to 1 week, up to 2 weeks, up to 3 weeks, up to 1 month, up to 2 months, up to 3 months, up to 4 months, up to 5 months, up to 6 months, up to 7 months, up to 8 months, up to 9 months, up to 10 months, or up to 12 months.

In some embodiments, the compositions of the present disclosure may be in liquid form. In some embodiments, the compositions of the present disclosure may be in solid form.

A composition of the present disclosure can be administered by various methods. For instance, in some embodiments, the administration occurs by methods that include, without limitation, intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, or combinations thereof.

In some embodiments, a composition of the present disclosure can be administered (e.g., via intranasal administration) in a single dose. In some embodiments, a method of the present disclosure can comprise administering the dose of a composition described herein (e.g., via intranasal administration) to a subject (e.g., an animal subject) at least 1 minute, at least 5 minutes, at least 10 minutes, at least 15 minutes, at least 20 minutes, at least 30 minutes, at least 45 minutes at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after exposure (or suspected exposure) to a pathogen that can cause a disease, such as a respiratory disease described herein. For example, a method for treating a subject having, at risk of having, or suspected of having been exposed to a pathogen that can cause a disease, such as a respiratory disease, can comprise administering one or more doses of a composition described herein (e.g., via intranasal administration) to a subject (e.g., an animal subject) at least 1 minute, at least 5 minutes, at least 10 minutes, at least 15 minutes, at least 20 minutes, at least 30 minutes, at least 45 minutes at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after exposure (or suspected exposure) to the pathogen. In some cases, a method for treating a subject having contracted a disease, such as a respiratory disease, from a pathogen (e.g., selected from the pathogens described herein) can comprise administering one or more doses of a composition described herein (e.g., via intranasal administration) to a subject (e.g., an animal subject) at least 1 minute, at least 5 minutes, at least 10 minutes, at least 15 minutes, at least 20 minutes, at least 30 minutes, at least 45 minutes, at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after exposure (or suspected exposure) to the pathogen. In some cases, a method for treating a subject having contracted a disease, such as a respiratory disease, caused by a pathogen (e.g., selected from the pathogens described herein) can comprise administering one or more doses of a composition described herein (e.g., via intranasal administration) to a subject (e.g., an animal subject) at least 1 minute, at least 5 minutes, at least 10 minutes, at least 15 minutes, at least 20 minutes, at least 30 minutes, at least 45 minutes, at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after clinical diagnosis of the disease in the subject.

In some cases, a method for slowing or stopping the rate of progression of a disease in a subject, such as a respiratory disease, caused by a pathogen (e.g., selected from the pathogens described herein) can comprise administering one or more doses of a composition described herein (e.g., via intranasal administration) to the subject (e.g., an animal subject) at least 1 minute, at least 5 minutes, at least 10 minutes, at least 15 minutes, at least 20 minutes, at least 30 minutes, at least 45 minutes at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after exposure (or suspected exposure) to the pathogen. In some cases, a method for slowing or stopping the rate of progression of a disease in a subject, such as a respiratory disease, caused by a pathogen (e.g., selected from the pathogens described herein) can comprise administering one or more doses of a composition described herein (e.g., via intranasal administration) to the subject (e.g., an animal subject) at least 1 minute, at least 5 minutes, at least 10 minutes, at least 15 minutes, at least 20 minutes, at least 30 minutes, at least 45 minutes at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after clinical diagnosis of the disease (or a stage, severity, or change in stage or severity thereof) in the subject.

In some cases, a method for reducing a risk of or preventing transmission of a pathogen (or disease state caused by the pathogen) from a first subject to a second subject can comprise administering one or more doses of a composition described herein (e.g., via intranasal administration) to the first subject (e.g., an animal subject) at least 1 minute, at least 5 minutes, at least 10 minutes, at least 15 minutes, at least 20 minutes, at least 30 minutes, at least 45 minutes at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after exposure (or suspected exposure) of the first subject to the pathogen. In some cases, a method for reducing a risk of or preventing transmission of a pathogen (or disease state caused by the pathogen) from a first subject to a second subject can comprise administering one or more doses of a composition described herein (e.g., via intranasal administration) to the second subject (e.g., an animal subject) at least 1 minute, at least 5 minutes, at least 10 minutes, at least 15 minutes, at least 20 minutes, at least 30 minutes, at least 45 minutes at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after clinical diagnosis of the disease (or a stage, severity, or change in stage or severity thereof) in the first subject. In some cases, a method for reducing a risk of or preventing transmission of a pathogen (or disease state caused by the pathogen) from a first subject to a second subject can comprise administering one or more doses of a composition described herein (e.g., via intranasal administration) to the second subject (e.g., an animal subject) at least 1 minute, at least 5 minutes, at least 10 minutes, at least 15 minutes, at least 20 minutes, at least 30 minutes, at least 45 minutes at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after exposure (or suspected exposure) of the first subject to the pathogen. In some cases, a method for reducing a risk of or preventing transmission of a pathogen (or disease state caused by the pathogen) from a first subject to a second subject can comprise administering one or more doses of a composition described herein (e.g., via intranasal administration) to the first subject (e.g., an animal subject) at least 1 minute, at least 5 minutes, at least 10 minutes, at least 15 minutes, at least 20 minutes, at least 30 minutes, at least 45 minutes, at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after clinical diagnosis of the disease (or a stage, severity, or change in stage or severity thereof) in the first subject.

In some cases, a method for reducing a risk of or preventing a subject from contracting a pathogen (or disease state caused by the pathogen) can comprise administering one or more doses of a composition described herein (e.g., via intranasal administration) to the first subject (e.g., an animal subject) at least 1 minute, at least 5 minutes, at least 10 minutes, at least 15 minutes, at least 20 minutes, at least 30 minutes, at least 45 minutes, at least 1 hour, at least 2 hours, at least 3 hours, at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days prior to the subject engaging in an activity associated with an increased risk of exposure to the pathogen.

In some embodiments, the therapeutic compositions of the present disclosure can be administered in multiple doses. In some embodiments, a method of treating or preventing a disease in a subject (e.g., an animal subject) can comprise administering 1, 2, 3, 4, 5, 6, 7, 8, 9, 10, 11, 12, 13, 14, 15, 15 to 20, 20 to 25, 25 to 30, 30 to 40, 40 to 50, or more than 50 doses. In some embodiments, a composition of the present disclosure may be administered to a subject (e.g., an animal subject) at a plurality of time points (e.g., at 1, 2, 3, 4, 5, 6, 7, 8, 9, 10, or more than 10 time points). In some embodiments, a method of the present disclosure can comprise administering to a subject a first dose comprising a composition of the present disclosure and a second dose comprising a composition of the present disclosure. In some embodiments, the composition of the first dose is the same as the composition of the second dose. In some embodiments, the composition of the first dose is different than the composition of the second dose. In some embodiments, a method of the present disclosure can comprise administering the first dose and the second dose to a subject (e.g., an animal subject) at an interval of at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days. In some embodiments, a method of the present disclosure can comprise administering the first dose and the second dose to a subject (e.g., an animal subject) at an interval of at most 6 hours, at most 12 hours, at most 24 hours, at most 36 hours, at most 2 days, at most 3 days, at most 4 days, at most 5 days, at most 6 days, at most 7 days, at most 14 days, or at most 28 days. In some embodiments, a method of the present disclosure can comprise administering the first dose and the second dose to a subject (e.g., an animal subject) at an interval of 6 hours, 12 hours, 24 hours, 36 hours, 2 days, 3 days, 4 days, 5 days, 6 days, 7 days, 14 days, or 28 days. In some embodiments, a method of the present disclosure can comprise administering each dose subsequent to a first dose of a plurality of doses (e.g., wherein each dose of the plurality of doses comprises one or more compositions of the present disclosure) to a subject (e.g., an animal subject) at least 4 hours, at least 6 hours, at least 12 hours, at least 24 hours, at least 36 hours, at least 2 days, at least 3 days, at least 4 days, at least 5 days, at least 6 days, at least 7 days, at least 14 days, or at least 28 days after administration of the previous dose. In some embodiments, a method of the present disclosure can comprise administering each dose subsequent to a first dose of a plurality of doses (e.g., wherein each dose of the plurality of doses comprises one or more compositions of the present disclosure) to a subject (e.g., an animal subject) at most 6 hours, at most 12 hours, at most 24 hours, at most 36 hours, at most 2 days, at most 3 days, at most 4 days, at most 5 days, at most 6 days, at most 7 days, at most 14 days, or at most 28 days after administration of the previous dose. In some embodiments, a method of the present disclosure can comprise administering each dose subsequent to a first dose of a plurality of doses (e.g., wherein each dose of the plurality of doses comprises one or more compositions of the present disclosure) to a subject (e.g., an animal subject) 6 hours, 12 hours, 24 hours, 36 hours, 2 days, 3 days, 4 days, 5 days, 6 days, 7 days, 14 days, or 28 days after administration of the previous dose. In some embodiments, the composition is administered to the subject in two, three, four, five, or more than five doses. In some embodiments, the second, third, and fourth doses (e.g., of a four-dose treatment) are administered 12 to 36 hours after the previous dose. In some embodiments, the second, third, and fourth doses (e.g., of a four-dose treatment) are administered 1, 2, 4, 6, 8, 10, 12, 18, 24, 30, or 36 hours after the previous dose.

In some embodiments, a compositions described herein (e.g., “NanoSTING” compositions comprising a lipid-based particle, a modulator, and an antigen) can be formulated and/or administered as a monotherapy (e.g., for treating a disease or condition in a subject, for example, which has resulted from exposure to a pathogen).

A composition or method described herein (e.g., for treating a disease or condition in a subject, for preventing a disease or condition in a subject, or for preventing transmission of a disease or condition from a first subject to a second subject) can comprise a nanoparticle (e.g., a lipid-based nanoparticle) and a modulator (e.g., a pattern recognition receptor agonist protein, such as a STING agonist). In some embodiments, a composition or method comprising a nanoparticle (e.g., a lipid-based nanoparticle) and a modulator may lack an antigen. In some embodiments, a modulator of a composition or method comprising a nanoparticle and a modulator but lacking an antigen can be encapsulated within the nanoparticle. In some embodiments, a composition comprising a nanoparticle and a modulator but lacking an antigen can be administered to a subject (e.g., a human subject or a non-human mammalian subject) in a dose, dosage form, and or a treatment or vaccination time course described herein. For example, a composition comprising a nanoparticle and a modulator but lacking an antigen can be administered to a subject before the subject (e.g., a human subject or a non-human mammalian subject) has been exposed to an infectious agent (e.g., a virus, such as a respiratory virus like a coronavirus, an influenza virus, a parainfluenza virus, or a rhinovirus). In some embodiments, a composition comprising a nanoparticle and a modulator but lacking an antigen can be administered to a subject (e.g., a human subject or a non-human mammalian subject) before the subject has contracted and/or become symptomatic of a respiratory disease or condition (e.g., associated with a respiratory virus) or, in some embodiments, a cancer such as a lung cancer. In some embodiments, a composition comprising a nanoparticle and a modulator but lacking an antigen can be administered to a subject (e.g., a human subject or a non-human mammalian subject) after the subject has been exposed to an infectious agent (e.g., a virus, such as a respiratory virus like a coronavirus, an influenza virus, a parainfluenza virus, or a rhinovirus). In some embodiments, a composition comprising a nanoparticle and a modulator but lacking an antigen can be administered to a subject (e.g., a human subject or a non-human mammalian subject) after the subject has contracted and/or become symptomatic of a respiratory disease or condition (e.g., associated with a respiratory virus) or, in some embodiments, a cancer such as a lung cancer. In some embodiments, a composition comprising a nanoparticle and a modulator but lacking an antigen can be administered to a subject intranasally. In some embodiments, compositions and methods comprising a nanoparticle and a modulator but lacking an antigen can be relatively inexpensive or logistically uncomplicated to manufacture, store, and/or prepare. In some embodiments, a composition comprising a nanoparticle and a modulator but lacking an antigen can be administered to a subject in combination with a composition comprising a nanoparticle, a modulator, and an antigen, for example, at the same time, sequentially, or separately as part of a single treatment or vaccination regimen.

A composition or method described herein (e.g., for treating a disease or condition in a subject, for preventing a disease or condition in a subject, or for preventing transmission of a disease or condition from a first subject to a second subject) can comprise a modulator described herein (e.g., a pattern recognition receptor agonist protein, such as a STING agonist). In some embodiments, a composition or method comprising a modulator may lack an antigen and a nanoparticle (e.g., liposome). In some embodiments, a modulator can be administered to a subject (e.g., a human subject or a non-human mammalian subject) without an antigen or nanoparticle, for example, to treat or prevent a disease or condition in a subject (e.g., a cancer or a disease or condition associated with a respiratory virus or respiratory disease). In some embodiments, a composition comprising a modulator but lacking an antigen or a nanoparticle can be administered to a subject (e.g., a human subject or a non-human mammalian subject) in a dose, dosage form, and or a treatment or vaccination time course described herein. For example, a composition comprising a modulator but lacking an antigen or a nanoparticle can be administered to a subject before the subject (e.g., a human subject or a non-human mammalian subject) has been exposed to an infectious agent (e.g., a virus, such as a respiratory virus like a coronavirus, an influenza virus, a parainfluenza virus, or a rhinovirus). In some embodiments, a composition comprising a modulator but lacking an antigen or a nanoparticle can be administered to a subject (e.g., a human subject or a non-human mammalian subject) before the subject has contracted and/or become symptomatic of a respiratory disease or condition (e.g., associated with a respiratory virus) or, in some embodiments, a cancer such as a lung cancer. In some embodiments, a composition comprising a modulator but lacking an antigen or a nanoparticle can be administered to a subject (e.g., a human subject or a non-human mammalian subject) after the subject has been exposed to an infectious agent (e.g., a virus, such as a respiratory virus like a coronavirus, an influenza virus, a parainfluenza virus, or a rhinovirus). In some embodiments, a composition comprising a modulator but lacking an antigen or nanoparticle can be administered to a subject (e.g., a human subject or a non-human mammalian subject) after the subject has contracted and/or become symptomatic of a respiratory disease or condition (e.g., associated with a respiratory virus) or, in some embodiments, a cancer such as a lung cancer. In some embodiments, a composition comprising a modulator but lacking an antigen or a nanoparticle can be administered to a subject intranasally. In some embodiments, compositions and methods comprising a modulator but lacking an antigen or a nanoparticle can be relatively inexpensive or logistically uncomplicated to manufacture, store, and/or prepare. In some embodiments, a composition comprising a nanoparticle and a modulator but lacking an antigen can be administered to a subject in combination with a composition comprising a nanoparticle, a modulator, and an antigen, for example, at the same time, sequentially, or separately as part of a single treatment or vaccination regimen.

In some embodiments, a composition of the present disclosure (e.g., for treating a subject having a disease or condition, such as a respiratory disease, or preventing the subject from contracting said disease or condition or transmitting said disease or condition to a second subject) can comprise 0.1 micrograms to 200 micrograms of a modulator (e.g., a STING pathway activator (e.g., a STING agonist)). In some embodiments, a composition of the present disclosure can comprise 0.1 micrograms to 1 microgram, 0.1 micrograms to 2 micrograms, 0.1 micrograms to 3 micrograms, 0.1 micrograms to 5 micrograms, 0.1 micrograms to 10 micrograms, 0.1 micrograms to 20 micrograms, 0.1 micrograms to 30 micrograms, 0.1 micrograms to 50 micrograms, 0.1 micrograms to 100 micrograms, 0.1 micrograms to 120 micrograms, 0.1 micrograms to 200 micrograms, 1 microgram to 2 micrograms, 1 microgram to 3 micrograms, 1 microgram to 5 micrograms, 1 microgram to 10 micrograms, 1 microgram to 20 micrograms, 1 microgram to 30 micrograms, 1 microgram to 50 micrograms, 1 microgram to 100 micrograms, 1 microgram to 120 micrograms, 1 microgram to 200 micrograms, 2 micrograms to 3 micrograms, 2 micrograms to 5 micrograms, 2 micrograms to 10 micrograms, 2 micrograms to 20 micrograms, 2 micrograms to 30 micrograms, 2 micrograms to 50 micrograms, 2 micrograms to 100 micrograms, 2 micrograms to 120 micrograms, 2 micrograms to 200 micrograms, 3 micrograms to 5 micrograms, 3 micrograms to 10 micrograms, 3 micrograms to 20 micrograms, 3 micrograms to 30 micrograms, 3 micrograms to 50 micrograms, 3 micrograms to 100 micrograms, 3 micrograms to 120 micrograms, 3 micrograms to 200 micrograms, 5 micrograms to 10 micrograms, 5 micrograms to 20 micrograms, 5 micrograms to 30 micrograms, 5 micrograms to 50 micrograms, 5 micrograms to 100 micrograms, 5 micrograms to 120 micrograms, 5 micrograms to 200 micrograms, 10 micrograms to 20 micrograms, 10 micrograms to 30 micrograms, 10 micrograms to 50 micrograms, 10 micrograms to 100 micrograms, 10 micrograms to 120 micrograms, 10 micrograms to 200 micrograms, 20 micrograms to 30 micrograms, 20 micrograms to 50 micrograms, 20 micrograms to 100 micrograms, 20 micrograms to 120 micrograms, 20 micrograms to 200 micrograms, 30 micrograms to 50 micrograms, 30 micrograms to 100 micrograms, 30 micrograms to 120 micrograms, 30 micrograms to 200 micrograms, 50 micrograms to 100 micrograms, 50 micrograms to 120 micrograms, 50 micrograms to 200 micrograms, 100 micrograms to 120 micrograms, 100 micrograms to 200 micrograms, or 120 micrograms to 200 micrograms of a modulator (e.g., a STING pathway activator (e.g., a STING agonist)). In some embodiments, a composition of the present disclosure can comprise 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist)). In some embodiments, a composition of the present disclosure can comprise at least 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist)). In some embodiments, a composition of the present disclosure can comprise at most 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist)).

In some embodiments, a composition of the present disclosure (e.g., for treating a subject having a disease or condition, such as a respiratory disease, or preventing the subject from contracting said disease or condition or transmitting said disease or condition to a second subject) can comprise 0.1 micrograms to 200 micrograms of a particle (e.g., a lipid-based particle (e.g., liposome), such as a lipid-based nanoparticle). In some embodiments, a composition of the present disclosure can comprise 0.1 micrograms to 1 microgram, 0.1 micrograms to 2 micrograms, 0.1 micrograms to 3 micrograms, 0.1 micrograms to 5 micrograms, 0.1 micrograms to 10 micrograms, 0.1 micrograms to 20 micrograms, 0.1 micrograms to 30 micrograms, 0.1 micrograms to 50 micrograms, 0.1 micrograms to 100 micrograms, 0.1 micrograms to 120 micrograms, 0.1 micrograms to 200 micrograms, 1 microgram to 2 micrograms, 1 microgram to 3 micrograms, 1 microgram to 5 micrograms, 1 microgram to 10 micrograms, 1 microgram to 20 micrograms, 1 microgram to 30 micrograms, 1 microgram to 50 micrograms, 1 microgram to 100 micrograms, 1 microgram to 120 micrograms, 1 microgram to 200 micrograms, 2 micrograms to 3 micrograms, 2 micrograms to 5 micrograms, 2 micrograms to 10 micrograms, 2 micrograms to 20 micrograms, 2 micrograms to 30 micrograms, 2 micrograms to 50 micrograms, 2 micrograms to 100 micrograms, 2 micrograms to 120 micrograms, 2 micrograms to 200 micrograms, 3 micrograms to 5 micrograms, 3 micrograms to 10 micrograms, 3 micrograms to 20 micrograms, 3 micrograms to 30 micrograms, 3 micrograms to 50 micrograms, 3 micrograms to 100 micrograms, 3 micrograms to 120 micrograms, 3 micrograms to 200 micrograms, 5 micrograms to 10 micrograms, 5 micrograms to 20 micrograms, 5 micrograms to 30 micrograms, 5 micrograms to 50 micrograms, 5 micrograms to 100 micrograms, 5 micrograms to 120 micrograms, 5 micrograms to 200 micrograms, 10 micrograms to 20 micrograms, 10 micrograms to 30 micrograms, 10 micrograms to 50 micrograms, 10 micrograms to 100 micrograms, 10 micrograms to 120 micrograms, 10 micrograms to 200 micrograms, 20 micrograms to 30 micrograms, 20 micrograms to 50 micrograms, 20 micrograms to 100 micrograms, 20 micrograms to 120 micrograms, 20 micrograms to 200 micrograms, 30 micrograms to 50 micrograms, 30 micrograms to 100 micrograms, 30 micrograms to 120 micrograms, 30 micrograms to 200 micrograms, 50 micrograms to 100 micrograms, 50 micrograms to 120 micrograms, 50 micrograms to 200 micrograms, 100 micrograms to 120 micrograms, 100 micrograms to 200 micrograms, or 120 micrograms to 200 micrograms of a particle (e.g., a lipid-based particle (e.g., liposome), such as a lipid-based nanoparticle). In some embodiments, a composition of the present disclosure can comprise 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a particle (e.g., a lipid-based particle (e.g., liposome), such as a lipid-based nanoparticle). In some embodiments, a composition of the present disclosure can comprise at least 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a particle (e.g., a lipid-based particle (e.g., liposome), such as a lipid-based nanoparticle). In some embodiments, a composition of the present disclosure can comprise at most 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a particle (e.g., a lipid-based particle (e.g., liposome), such as a lipid-based nanoparticle).

In some embodiments, a composition of the present disclosure (e.g., for treating a subject having a disease or condition, such as a respiratory disease, or preventing the subject from contracting said disease or condition or transmitting said disease or condition to a second subject) comprising 20 micrograms of a particle (e.g., a lipid-based particle (e.g., liposome), such as a lipid-based nanoparticle) can comprise 0.1 micrograms to 200 micrograms of a modulator (e.g., a STING pathway activator (e.g., a STING agonist)). In some embodiments, a composition of the present disclosure comprising 20 micrograms of a particle (e.g., a lipid-based particle (e.g., liposome), such as a lipid-based nanoparticle) can comprise 0.1 micrograms to 1 microgram, 0.1 micrograms to 2 micrograms, 0.1 micrograms to 3 micrograms, 0.1 micrograms to 5 micrograms, 0.1 micrograms to 10 micrograms, 0.1 micrograms to 20 micrograms, 0.1 micrograms to 30 micrograms, 0.1 micrograms to 50 micrograms, 0.1 micrograms to 100 micrograms, 0.1 micrograms to 120 micrograms, 0.1 micrograms to 200 micrograms, 1 microgram to 2 micrograms, 1 microgram to 3 micrograms, 1 microgram to 5 micrograms, 1 microgram to 10 micrograms, 1 microgram to 20 micrograms, 1 microgram to 30 micrograms, 1 microgram to 50 micrograms, 1 microgram to 100 micrograms, 1 microgram to 120 micrograms, 1 microgram to 200 micrograms, 2 micrograms to 3 micrograms, 2 micrograms to 5 micrograms, 2 micrograms to 10 micrograms, 2 micrograms to 20 micrograms, 2 micrograms to 30 micrograms, 2 micrograms to 50 micrograms, 2 micrograms to 100 micrograms, 2 micrograms to 120 micrograms, 2 micrograms to 200 micrograms, 3 micrograms to 5 micrograms, 3 micrograms to 10 micrograms, 3 micrograms to 20 micrograms, 3 micrograms to 30 micrograms, 3 micrograms to 50 micrograms, 3 micrograms to 100 micrograms, 3 micrograms to 120 micrograms, 3 micrograms to 200 micrograms, 5 micrograms to 10 micrograms, 5 micrograms to 20 micrograms, 5 micrograms to 30 micrograms, 5 micrograms to 50 micrograms, 5 micrograms to 100 micrograms, 5 micrograms to 120 micrograms, 5 micrograms to 200 micrograms, 10 micrograms to 20 micrograms, 10 micrograms to 30 micrograms, 10 micrograms to 50 micrograms, 10 micrograms to 100 micrograms, 10 micrograms to 120 micrograms, 10 micrograms to 200 micrograms, 20 micrograms to 30 micrograms, 20 micrograms to 50 micrograms, 20 micrograms to 100 micrograms, 20 micrograms to 120 micrograms, 20 micrograms to 200 micrograms, 30 micrograms to 50 micrograms, 30 micrograms to 100 micrograms, 30 micrograms to 120 micrograms, 30 micrograms to 200 micrograms, 50 micrograms to 100 micrograms, 50 micrograms to 120 micrograms, 50 micrograms to 200 micrograms, 100 micrograms to 120 micrograms, 100 micrograms to 200 micrograms, or 120 micrograms to 200 micrograms of a modulator (e.g., a STING pathway activator (e.g., a STING agonist, such as cGAMP)). In some embodiments, a composition of the present disclosure comprising 20 micrograms of a particle (e.g., a lipid-based particle (e.g., liposome), such as a lipid-based nanoparticle) can comprise 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist, such as cGAMP)). In some embodiments, a composition of the present disclosure comprising 20 micrograms of a particle (e.g., a lipid-based particle (e.g., liposome), such as a lipid-based nanoparticle) can comprise at least 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist, such as cGAMP)). In some embodiments, a composition of the present disclosure comprising 20 micrograms of a particle (e.g., a lipid-based particle (e.g., liposome), such as a lipid-based nanoparticle) can comprise at most 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist, such as cGAMP)).

In some embodiments, a composition of the present disclosure can comprise a plurality of different antigens. For example, a composition of the present disclosure can comprise a nucleocapsid protein (N-protein) and an S-protein (e.g., a coronavirus spike protein). In some embodiments, a composition of the present disclosure can comprise a plurality of types of a single class of antigen. For instance, a composition of the present disclosure can comprise a monomeric antigen (e.g., a monomeric S-protein), a trimeric antigen (e.g., a trimeric S-protein), a chimeric antigen (e.g., a chimeric S-protein), and/or a variant of an antigen (e.g., a variant S-protein).

In some embodiments, a composition of the present disclosure (e.g., for treating a subject having a disease or condition, such as a respiratory disease, or preventing the subject from contracting said disease or condition or transmitting said disease or condition to a second subject) can comprise 0.1 micrograms to 200 micrograms of an antigen (e.g., a nucleocapsid protein (N-protein), a spike protein (S-protein), or an RSV antigen protein). In some embodiments, a composition of the present disclosure can comprise 0.1 micrograms to 1 microgram, 0.1 micrograms to 2 micrograms, 0.1 micrograms to 3 micrograms, 0.1 micrograms to 5 micrograms, 0.1 micrograms to 10 micrograms, 0.1 micrograms to 20 micrograms, 0.1 micrograms to 30 micrograms, 0.1 micrograms to 50 micrograms, 0.1 micrograms to 100 micrograms, 0.1 micrograms to 120 micrograms, 0.1 micrograms to 200 micrograms, 1 microgram to 2 micrograms, 1 microgram to 3 micrograms, 1 microgram to 5 micrograms, 1 microgram to 10 micrograms, 1 microgram to 20 micrograms, 1 microgram to 30 micrograms, 1 microgram to 50 micrograms, 1 microgram to 100 micrograms, 1 microgram to 120 micrograms, 1 microgram to 200 micrograms, 2 micrograms to 3 micrograms, 2 micrograms to 5 micrograms, 2 micrograms to 10 micrograms, 2 micrograms to 20 micrograms, 2 micrograms to 30 micrograms, 2 micrograms to 50 micrograms, 2 micrograms to 100 micrograms, 2 micrograms to 120 micrograms, 2 micrograms to 200 micrograms, 3 micrograms to 5 micrograms, 3 micrograms to 10 micrograms, 3 micrograms to 20 micrograms, 3 micrograms to 30 micrograms, 3 micrograms to 50 micrograms, 3 micrograms to 100 micrograms, 3 micrograms to 120 micrograms, 3 micrograms to 200 micrograms, 5 micrograms to 10 micrograms, 5 micrograms to 20 micrograms, 5 micrograms to 30 micrograms, 5 micrograms to 50 micrograms, 5 micrograms to 100 micrograms, 5 micrograms to 120 micrograms, 5 micrograms to 200 micrograms, 10 micrograms to 20 micrograms, 10 micrograms to 30 micrograms, 10 micrograms to 50 micrograms, 10 micrograms to 100 micrograms, 10 micrograms to 120 micrograms, 10 micrograms to 200 micrograms, 20 micrograms to 30 micrograms, 20 micrograms to 50 micrograms, 20 micrograms to 100 micrograms, 20 micrograms to 120 micrograms, 20 micrograms to 200 micrograms, 30 micrograms to 50 micrograms, 30 micrograms to 100 micrograms, 30 micrograms to 120 micrograms, 30 micrograms to 200 micrograms, 50 micrograms to 100 micrograms, 50 micrograms to 120 micrograms, 50 micrograms to 200 micrograms, 100 micrograms to 120 micrograms, 100 micrograms to 200 micrograms, or 120 micrograms to 200 micrograms of an antigen (e.g., a nucleocapsid protein (N-protein), a spike protein (S-protein), or an RSV antigen protein). In some embodiments, a composition of the present disclosure can comprise 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of an antigen (e.g., a nucleocapsid protein (N-protein), a spike protein (S-protein), or an RSV antigen protein). In some embodiments, a composition of the present disclosure can comprise at least 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of an antigen (e.g., a nucleocapsid protein (N-protein), a spike protein (S-protein), or an RSV antigen protein). In some embodiments, a composition of the present disclosure can comprise at most 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of an antigen (e.g., a nucleocapsid protein (N-protein), a spike protein (S-protein), or an RSV antigen protein). In some embodiments, a composition of the present disclosure (e.g., for treating a subject having a disease or condition, such as a respiratory disease, or preventing the subject from contracting said disease or condition or transmitting said disease or condition to a second subject) comprising 20 micrograms of a particle (e.g., a lipid-based particle (e.g., liposome), such as a lipid-based nanoparticle) can comprise 0.1 micrograms to 200 micrograms of an antigen (e.g., a nucleocapsid protein (N-protein), a spike protein (S-protein), or an RSV antigen protein). In some embodiments, a composition of the present disclosure comprising 20 micrograms of a particle (e.g., a lipid-based particle (e.g., a liposome), such as a lipid-based nanoparticle) can comprise 0.1 micrograms to 1 microgram, 0.1 micrograms to 2 micrograms, 0.1 micrograms to 3 micrograms, 0.1 micrograms to 5 micrograms, 0.1 micrograms to 10 micrograms, 0.1 micrograms to 20 micrograms, 0.1 micrograms to 30 micrograms, 0.1 micrograms to 50 micrograms, 0.1 micrograms to 100 micrograms, 0.1 micrograms to 120 micrograms, 0.1 micrograms to 200 micrograms, 1 microgram to 2 micrograms, 1 microgram to 3 micrograms, 1 microgram to 5 micrograms, 1 microgram to 10 micrograms, 1 microgram to 20 micrograms, 1 microgram to 30 micrograms, 1 microgram to 50 micrograms, 1 microgram to 100 micrograms, 1 microgram to 120 micrograms, 1 microgram to 200 micrograms, 2 micrograms to 3 micrograms, 2 micrograms to 5 micrograms, 2 micrograms to 10 micrograms, 2 micrograms to 20 micrograms, 2 micrograms to 30 micrograms, 2 micrograms to 50 micrograms, 2 micrograms to 100 micrograms, 2 micrograms to 120 micrograms, 2 micrograms to 200 micrograms, 3 micrograms to 5 micrograms, 3 micrograms to 10 micrograms, 3 micrograms to 20 micrograms, 3 micrograms to 30 micrograms, 3 micrograms to 50 micrograms, 3 micrograms to 100 micrograms, 3 micrograms to 120 micrograms, 3 micrograms to 200 micrograms, 5 micrograms to 10 micrograms, 5 micrograms to 20 micrograms, 5 micrograms to 30 micrograms, 5 micrograms to 50 micrograms, 5 micrograms to 100 micrograms, 5 micrograms to 120 micrograms, 5 micrograms to 200 micrograms, 10 micrograms to 20 micrograms, 10 micrograms to 30 micrograms, 10 micrograms to 50 micrograms, 10 micrograms to 100 micrograms, 10 micrograms to 120 micrograms, 10 micrograms to 200 micrograms, 20 micrograms to 30 micrograms, 20 micrograms to 50 micrograms, 20 micrograms to 100 micrograms, 20 micrograms to 120 micrograms, 20 micrograms to 200 micrograms, 30 micrograms to 50 micrograms, 30 micrograms to 100 micrograms, 30 micrograms to 120 micrograms, 30 micrograms to 200 micrograms, 50 micrograms to 100 micrograms, 50 micrograms to 120 micrograms, 50 micrograms to 200 micrograms, 100 micrograms to 120 micrograms, 100 micrograms to 200 micrograms, or 120 micrograms to 200 micrograms of an antigen (e.g., a nucleocapsid protein (N-protein), a spike protein (S-protein), or an RSV antigen protein). In some embodiments, a composition of the present disclosure comprising 20 micrograms of a particle (e.g., a lipid-based particle (e.g., liposome), such as a lipid-based nanoparticle) can comprise 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of an antigen (e.g., a nucleocapsid protein (N-protein), a spike protein (S-protein), or an RSV antigen protein). In some embodiments, a composition of the present disclosure comprising 20 micrograms of a particle (e.g., a lipid-based particle (e.g., liposome), such as a lipid-based nanoparticle) can comprise at least 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of an antigen (e.g., a nucleocapsid protein (N-protein), a spike protein (S-protein), or an RSV antigen protein). In some embodiments, a composition of the present disclosure comprising 20 micrograms of a particle (e.g., a lipid-based particle (e.g., liposome), such as a lipid-based nanoparticle) can comprise at most 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of an antigen (e.g., a nucleocapsid protein (N-protein), a spike protein (S-protein), or an RSV antigen protein).

In some embodiments, a composition of the present disclosure (e.g., for treating a subject having a disease or condition, such as a respiratory disease, or preventing the subject from contracting said disease or condition or transmitting said disease or condition to a second subject) comprising at least 20 micrograms of a particle (e.g., a lipid-based particle (e.g., liposome), such as a lipid-based nanoparticle) and at least 4 micrograms of an antigen can comprise 0.1 micrograms to 200 micrograms of a modulator (e.g., a STING pathway activator (e.g., a STING agonist)). In some embodiments, a composition of the present disclosure comprising at least 20 micrograms of a particle (e.g., a lipid-based particle (e.g., liposome), such as a lipid-based nanoparticle) and at least 4 micrograms of an antigen can comprise 0.1 micrograms to 1 microgram, 0.1 micrograms to 2 micrograms, 0.1 micrograms to 3 micrograms, 0.1 micrograms to 5 micrograms, 0.1 micrograms to 10 micrograms, 0.1 micrograms to 20 micrograms, 0.1 micrograms to 30 micrograms, 0.1 micrograms to 50 micrograms, 0.1 micrograms to 100 micrograms, 0.1 micrograms to 120 micrograms, 0.1 micrograms to 200 micrograms, 1 microgram to 2 micrograms, 1 microgram to 3 micrograms, 1 microgram to 5 micrograms, 1 microgram to 10 micrograms, 1 microgram to 20 micrograms, 1 microgram to 30 micrograms, 1 microgram to 50 micrograms, 1 microgram to 100 micrograms, 1 microgram to 120 micrograms, 1 microgram to 200 micrograms, 2 micrograms to 3 micrograms, 2 micrograms to 5 micrograms, 2 micrograms to 10 micrograms, 2 micrograms to 20 micrograms, 2 micrograms to 30 micrograms, 2 micrograms to 50 micrograms, 2 micrograms to 100 micrograms, 2 micrograms to 120 micrograms, 2 micrograms to 200 micrograms, 3 micrograms to 5 micrograms, 3 micrograms to 10 micrograms, 3 micrograms to 20 micrograms, 3 micrograms to 30 micrograms, 3 micrograms to 50 micrograms, 3 micrograms to 100 micrograms, 3 micrograms to 120 micrograms, 3 micrograms to 200 micrograms, 5 micrograms to 10 micrograms, 5 micrograms to 20 micrograms, 5 micrograms to 30 micrograms, 5 micrograms to 50 micrograms, 5 micrograms to 100 micrograms, 5 micrograms to 120 micrograms, 5 micrograms to 200 micrograms, 10 micrograms to 20 micrograms, 10 micrograms to 30 micrograms, 10 micrograms to 50 micrograms, 10 micrograms to 100 micrograms, 10 micrograms to 120 micrograms, 10 micrograms to 200 micrograms, 20 micrograms to 30 micrograms, 20 micrograms to 50 micrograms, 20 micrograms to 100 micrograms, 20 micrograms to 120 micrograms, 20 micrograms to 200 micrograms, 30 micrograms to 50 micrograms, 30 micrograms to 100 micrograms, 30 micrograms to 120 micrograms, 30 micrograms to 200 micrograms, 50 micrograms to 100 micrograms, 50 micrograms to 120 micrograms, 50 micrograms to 200 micrograms, 100 micrograms to 120 micrograms, 100 micrograms to 200 micrograms, or 120 micrograms to 200 micrograms of a modulator (e.g., a STING pathway activator (e.g., a STING agonist)). In some embodiments, a composition of the present disclosure comprising at least 20 micrograms of a particle (e.g., a lipid-based particle (e.g., liposome), such as a lipid-based nanoparticle) and at least 4 micrograms of an antigen can comprise 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist)). In some embodiments, a composition of the present disclosure comprising at least 20 micrograms of a particle (e.g., a lipid-based particle (e.g., liposome), such as a lipid-based nanoparticle) and at least 4 micrograms of an antigen can comprise at least 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist)). In some embodiments, a composition of the present disclosure comprising at least 20 micrograms of a particle (e.g., a lipid-based particle (e.g., liposome), such as a lipid-based nanoparticle) and at least 4 micrograms of an antigen can comprise at most 0.1 micrograms, 1 microgram, 2 micrograms, 3 micrograms, 5 micrograms, 10 micrograms, 20 micrograms, 30 micrograms, 50 micrograms, 100 micrograms, 120 micrograms, or 200 micrograms of a modulator (e.g., a STING pathway activator (e.g., a STING agonist)).

In some embodiments, a dose administered to a subject according to a composition or method described herein can comprise 0.1 micrograms to 20 micrograms of a composition described herein (e.g., comprising a modulator and a particle or comprising a modulator, a particle, and an antigen). In some embodiments, a dose administered to a subject according to a composition or method described herein can comprise 0.1 micrograms to 0.5 micrograms, 0.1 micrograms to 1 microgram, 0.1 micrograms to 2.5 micrograms, 0.1 micrograms to 5 micrograms, 0.1 micrograms to 7.5 micrograms, 0.1 micrograms to 9.3 micrograms, 0.1 micrograms to 10 micrograms, 0.1 micrograms to 15 micrograms, 0.1 micrograms to 20 micrograms, 0.5 micrograms to 1 microgram, 0.5 micrograms to 2.5 micrograms, 0.5 micrograms to 5 micrograms, 0.5 micrograms to 7.5 micrograms, 0.5 micrograms to 9.3 micrograms, 0.5 micrograms to 10 micrograms, 0.5 micrograms to 15 micrograms, 0.5 micrograms to 20 micrograms, 1 microgram to 2.5 micrograms, 1 microgram to 5 micrograms, 1 microgram to 7.5 micrograms, 1 microgram to 9.3 micrograms, 1 microgram to 10 micrograms, 1 microgram to 15 micrograms, 1 microgram to 20 micrograms, 2.5 micrograms to 5 micrograms, 2.5 micrograms to 7.5 micrograms, 2.5 micrograms to 9.3 micrograms, 2.5 micrograms to 10 micrograms, 2.5 micrograms to 15 micrograms, 2.5 micrograms to 20 micrograms, 5 micrograms to 7.5 micrograms, 5 micrograms to 9.3 micrograms, 5 micrograms to 10 micrograms, 5 micrograms to 15 micrograms, 5 micrograms to 20 micrograms, 7.5 micrograms to 9.3 micrograms, 7.5 micrograms to 10 micrograms, 7.5 micrograms to 15 micrograms, 7.5 micrograms to 20 micrograms, 9.3 micrograms to 10 micrograms, 9.3 micrograms to 15 micrograms, 9.3 micrograms to 20 micrograms, 10 micrograms to 15 micrograms, 10 micrograms to 20 micrograms, or 15 micrograms to 20 micrograms of a composition described herein (e.g., comprising a modulator and a particle or comprising a modulator, a particle, and an antigen). In some embodiments, a dose administered to a subject according to a composition or method described herein can comprise 0.1 micrograms, 0.5 micrograms, 1.0 microgram, 1.2 micrograms, 2.3 micrograms, 2.5 micrograms, 4.7 micrograms, 5.0 micrograms, 7.5 micrograms, 9.3 micrograms, 10 micrograms, 15 micrograms, or 20 micrograms of a composition described herein (e.g., comprising a modulator and a particle or comprising a modulator, a particle, and an antigen). In some embodiments, a dose administered to a subject according to a composition or method described herein can comprise at least 0.1 micrograms, 0.5 micrograms, 1.0 microgram, 1.2 micrograms, 2.3 micrograms, 2.5 micrograms, 4.7 micrograms, 5.0 micrograms, 7.5 micrograms, 9.3 micrograms, 10 micrograms, 15 micrograms, or 20 micrograms of a composition described herein (e.g., comprising a modulator and a particle or comprising a modulator, a particle, and an antigen). In some embodiments, a dose administered to a subject according to a composition or method described herein can comprise at most 0.1 micrograms, 0.5 micrograms, 1.0 microgram, 1.2 micrograms, 2.3 micrograms, 2.5 micrograms, 4.7 micrograms, 5.0 micrograms, 7.5 micrograms, 9.3 micrograms, 10 micrograms, 15 micrograms, or 20 micrograms of a composition described herein (e.g., comprising a modulator and a particle or comprising a modulator, a particle, and an antigen).

In some embodiments, a composition of the present disclosure can be administered in combination with other therapeutic treatments. In some embodiments, the other therapeutic treatments include, without limitation, immunotherapy, virotherapy, targeted inhibition, radiotherapy, chemotherapy, or combinations thereof. In some embodiments, a composition described herein can comprise and/or can be administered in a treatment regimen (e.g., administered concurrently or non-concurrently) with an adjuvant (e.g., one or more antibodies, one or more antiviral treatments, one or more vaccines, one or more small molecules, one or more nucleic acids, and/or one or more peptides or proteins, such as an interleukin (e.g., IL-21)).

### Subjects

The therapeutic compositions of the present disclosure may be administered to subjects in need thereof. For instance, in some embodiments, the subject is a mammal (e.g., a human). In some embodiments, the subject is a domesticated animal. For example, the subject can be a dog or a cat. In some embodiments, a subject can be a cow, a horse, a non-human primate, a mouse, a rat, a rabbit, a guinea pig, a goat, a sheep, a giraffe, a zebra, a lion, a tiger, or a bear. In some embodiments, the subject can be a human being. In some embodiments, the subject can be vulnerable to or suffering from a respiratory infection. In some embodiments, the subject is vulnerable to or suffering from a condition caused by a respiratory virus. For example, a composition or method can be administered to a subject who has been exposed to or who has been infected with a respiratory virus, for example a respiratory RNA virus, such as a coronavirus (e.g., an alpha variant, a delta variant, or an omicron variant), an influenza virus (e.g., influenza A), a respiratory syncytial virus, a metapneumovirus, a parainfluenza virus, or a rhinovirus. In some embodiments, the subject can be vulnerable to or suffering from a cancer. In some embodiments, a subject can be selected for treatment as a result of exhibiting one or more symptoms of infection from a pathogen or contraction of a disease (e.g., infection by a respiratory pathogen or contraction of a respiratory disease). For example, a subject may be selected by for treatment based on having one or more symptoms, including persistent coughing, elevated body temperature (e.g., greater than 100.4° C. by forehead skin measurement), body chills, achy joints, difficulty breathing or catching one's breath, fluid in the lungs, fatigue, headache, loss of taste or smell, or a positive diagnostic test, such as a PCR test. In some embodiments, a subject may be selected for treatment with a composition disclosed herein based on a demographic risk factor, such as obesity, advanced age (e.g., 65 years old or older), immune impairment, or pregnancy. In some embodiments, a subject may be selected for treatment based on a risk of infection by a pathogen or contraction of a disease (e.g., infection by a respiratory pathogen or contraction of a respiratory disease), for instance, if the subject has an occupation involving close interaction with customers, frequent interaction with at-risk populations, handling of biological samples, or close contact with potentially infected individuals.

### Treatment or Prevention of Diseases

Pathogens and cancers have caused significant health and economic concerns. For instance, viral infections caused by severe acute respiratory syndrome-related coronavirus 2 (SARS-CoV-2) have resulted in the COVID-19 pandemic, which is currently the most urgent health and economic crisis in the world. Moreover, cancers such as lung cancer have high fatality rates.

Additionally, very limited therapeutic options exist for effectively treating and preventing cancers, and infections caused by pathogens. For instance, no medicine is currently available for treating or preventing viral infections caused by SARS-CoV-2.

Additionally, many vaccines that aim to prevent infections caused by pathogens are intramuscular vaccines that are designed to elicit systemic immunity without conferring innate immunity, such as mucosal immunity or broad T-cell immunity. Such intramuscular vaccines present limitations because the nasal compartment is the first barrier that needs to be breached by pathogens before dissemination to the lung.

Furthermore, a high level of unpredictability exists in the effectiveness of vaccine formulations. For vaccines that make it to Phase I trials, only ˜⅓ are able to obtain eventual FDA approval. This rate of success implies that, even with existing technologies to prime the immune system, the development of effective immunity to every pathogen requires design and optimization of the correct antigen and the adjuvant.

Even achieving FDA approval does not imply that a vaccine is efficacious. Developing vaccines is a lengthy process and, even in the context of the COVID19 pandemic, there are a number of challenges.

Even though the majority of platforms target the spike protein as the antigen, optimizing the antigen design is desirable to ensure the efficacy of the vaccine. The difficulty in picking the appropriate vaccine design has been highlighted with HIV-1, Hepatitis C and malaria, wherein, despite decades of efforts and multiple candidates, vaccines remain elusive.

Moreover, with respect to respiratory pathogens, the nasal compartment is the first barrier that needs to be breached by the pathogens before dissemination to the lung. However, current intramuscular vaccines are designed to elicit systemic immunity without conferring innate immunity, such as mucosal immunity.

Accordingly, safe and durable therapeutic compositions are urgently needed to treat and prevent various diseases, such as infections caused by pathogens and various types of cancer. For instance, safe and durable therapeutic compositions are urgently needed to treat or prevent pandemics caused by respiratory pathogens (e.g., viral infections caused by coronaviruses, such as SARS-CoV-2). Numerous embodiments of the present disclosure address the aforementioned needs.

The therapeutic compositions and methods of the present disclosure can be utilized to treat or prevent a disease or condition (e.g., a disease or condition associated with a pathogen). For instance, in some embodiments, the disease can be an infection caused by a pathogen. In some embodiments, the disease can be a respiratory disease. In some embodiments, the pathogen can be a respiratory pathogen. In some embodiments, the respiratory pathogen can be a virus. In some embodiments, the virus can be an influenza virus, an influenza A virus (e.g., California/04/2009(H1N1) or Hong Kong/2369/2009(H1N1)), an influenza B virus, a parainfluenza virus, an adenovirus, an enterovirus, a coronavirus, a respiratory syncytial virus, a rhinovirus, a DNA virus, an RNA virus, variants thereof, or a combination thereof.

In some embodiments, the pathogen is a coronavirus. In some embodiments, the coronavirus includes, without limitation, severe acute respiratory syndrome coronavirus (SARS-CoV), severe acute respiratory syndrome-related coronavirus (SARSr-CoV), human coronavirus 229E (HCoV-229E), human coronavirus NL63 (HCoV-NL63), human coronavirus 0C43 (HCoV-0C43), human coronavirus HKU1 (HCoV-HKU1), Middle East respiratory syndrome-related coronavirus (MERS-CoV), severe acute respiratory syndrome-related coronavirus 2 (SARS-CoV-2), variants of SARS-CoV-2 (e.g., 20A.EU1, or spike variant D614G), or combinations thereof. In some embodiments, the coronavirus is a SARS-CoV-2 virus, an alpha variant thereof (e.g., B.1.1.7), a delta variant thereof (e.g., B.1.617.2), an omicron variant thereof (e.g., B.1.1.529), or combinations thereof.

In some embodiments, the pathogen is an influenza virus. In some embodiments, the influenza virus includes an oseltamivir-sensitive strain, a treatment resistant strain, or a combination thereof.

In some embodiments of the compositions and methods, a subject (e.g., a mammalian subject such as a human, mouse, hamster, canine, feline, or non-human primate) can be prophylactically treated to protect against viral infection (e.g., SARS-CoV-2 coronavirus) by intranasal delivery of a composition described herein (e.g., comprising a modulator, such as a STING agonist and a lipid-based nanoparticle) and an antigen (e.g., a spike protein associated with, embedded in, or coupled to an outer surface of the lipid-based nanoparticle), a single-dose or as part of a multi-dose regimen. In some embodiments, treatment with compositions can elicit an immune response in a subject in one or more body compartments, including the blood, spleen, lung, and/or nasal compartment. In some embodiments, compositions and methods described herein can elicit increased anti-antigen IgG (e.g., anti-spike protein IgG) and/or anti-antigen IgA (e.g., anti-spike protein IgA) detectable in lung tissue (e.g., BALF) or blood (e.g., serum) of a subject. In some embodiments, compositions and methods described herein can increase the number of IgA-secreting B cells and/or the number of antigen-specific T cells (e.g., spike protein-specific T cells), for example, in the spleen. In some embodiments, an increase in antigen-specific T cells can be detected in the lung after administration of a composition described herein to a subject. In some embodiments, increased anti-antigen IgA and/or increased presence and/or activity of germinal center (GC) B cells or T follicular helper (Tfh) cells can be detected in the nasal compartment after administration of a composition described herein to a subject.

In some embodiments, the disease to be treated or prevented in a subject is a cancer. In some embodiments, the cancer can be tracheal cancer, lung cancer, bronchial cancer, epithelial cancer, blood cancer, breast cancer, melanoma, ovarian cancer, gynecological cancer, a leukemia, a lymphoma, prostate cancer, bladder cancer, colon cancer, a glioma, a sarcoma, glioblastoma, or a combination thereof. In some embodiments, the cancer can be lung cancer.

### EXAMPLES

### Example 1: Preparation, Characterization, and Stability of NanoSTING

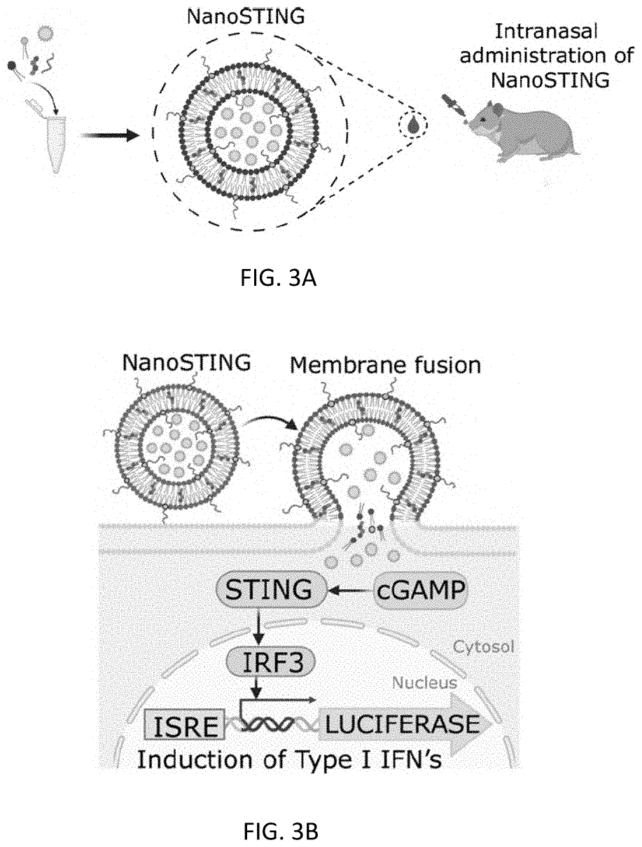

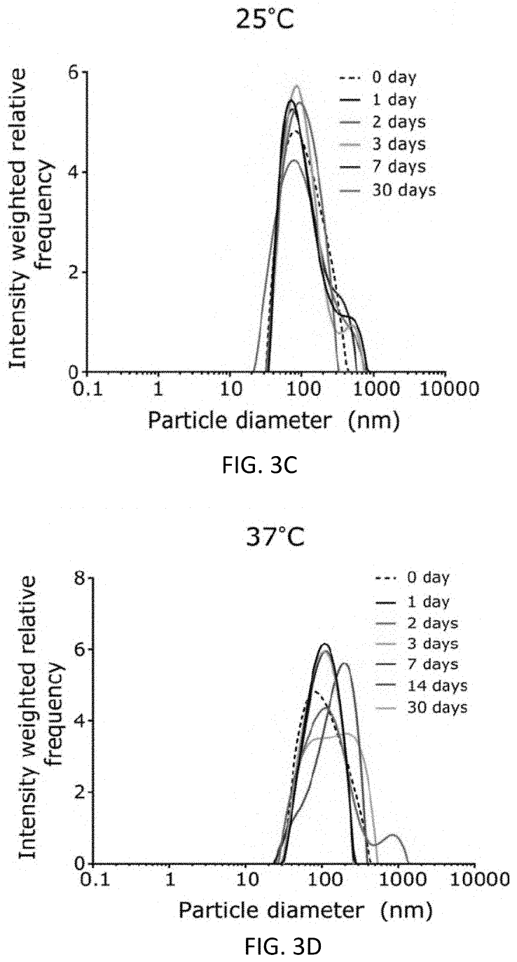

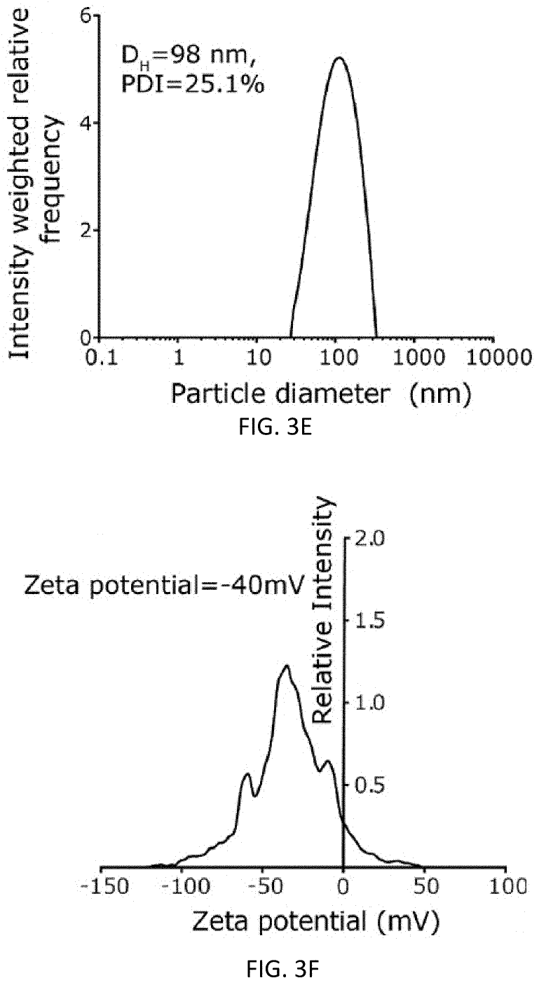

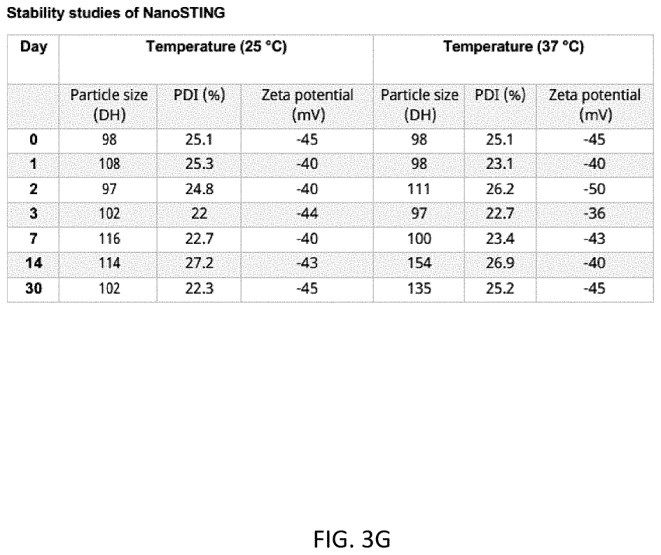

This example describes the preparation, characterization, and stability of NanoSTING, a liposomal formulation containing the natural immunotransmitter, cyclic GMP-AMP, and cGAMP (FIG. 3A).

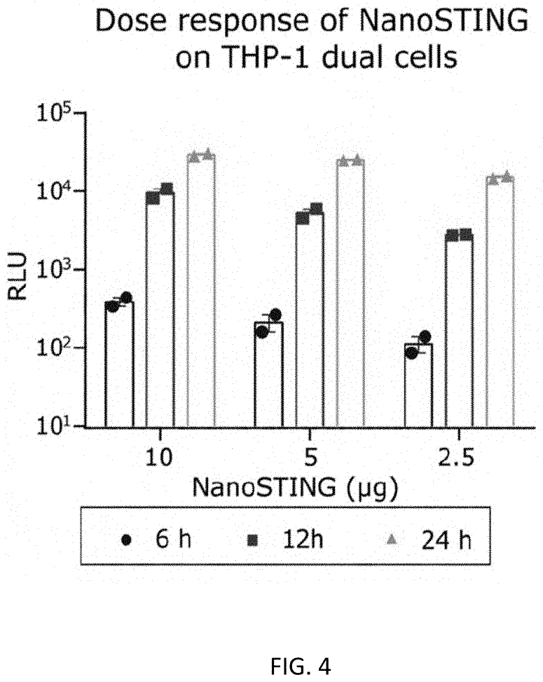

The nanoparticles promoted stability and delivery to alveolar macrophages, facilitating responses in the upper airways and the lung. Dynamic light scattering (DLS) analysis revealed that the mean particle diameter of NanoSTING was 98 nm, with a polydispersity index of 0.25 (FIG. 3E). The zeta potential of NanoSTING was −40 mV (FIG. 3F). Administration of NanoSTING was able to induce interferon responses by using THP-1 monocytic cells modified to conditionally secrete luciferase downstream of an Interferon regulatory factor (IRF) responsive promoter (FIG. 3B). THP-1 cells with NanoSTING were administered at doses ranging from 2.5-10.0 μg and kinetic measurements were performed for 24 h by measuring the luciferase activity in the supernatant. A low level of luciferase activity at 6 h was observed, and secretion was maximal at 24 h with 5, and 10 μg NanoSTING (FIG. 4). The longitudinal nanoparticle stability of NanoSTING was measured at two different temperatures, 25° C. and 37° C. DLS was then used to track instability and zeta potential was used to assess the change in charge of the nanoparticles. While the hydrodynamic diameter of NanoSTING was essentially unchanged at 25° C. over a period of 30 days (FIG. 3C), there was a slight increase in hydrodynamic diameter at 37° C. after 2 weeks (mean: 114 nm at 25° C. and 154 nm at 37° C.) (FIG. 3D). There was no significant change in zeta potential at both temperatures (−45 mV at 25° C. and at 37° C.). These results demonstrated that NanoSTING was immunologically active and nanoparticle remained stable even without refrigeration.

### Example 2: Sustained Interferon-Beta (IFNβ) Secretion in the Nasal Compartment Following NanoSTING Delivery

This example demonstrates the ability of NanoSTING to deliver cGAMP in the nasal compartment of mice and induce secretion of effector cytokines.

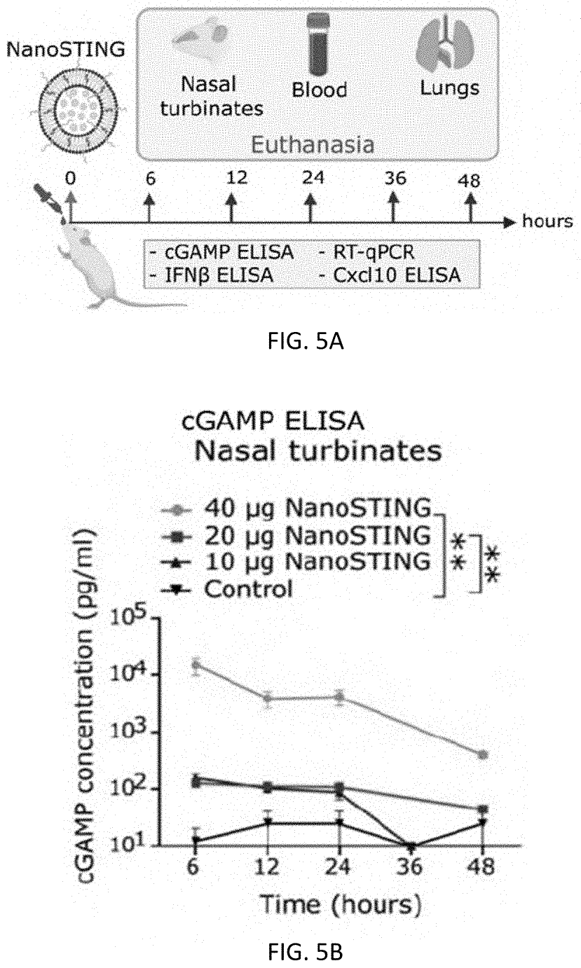

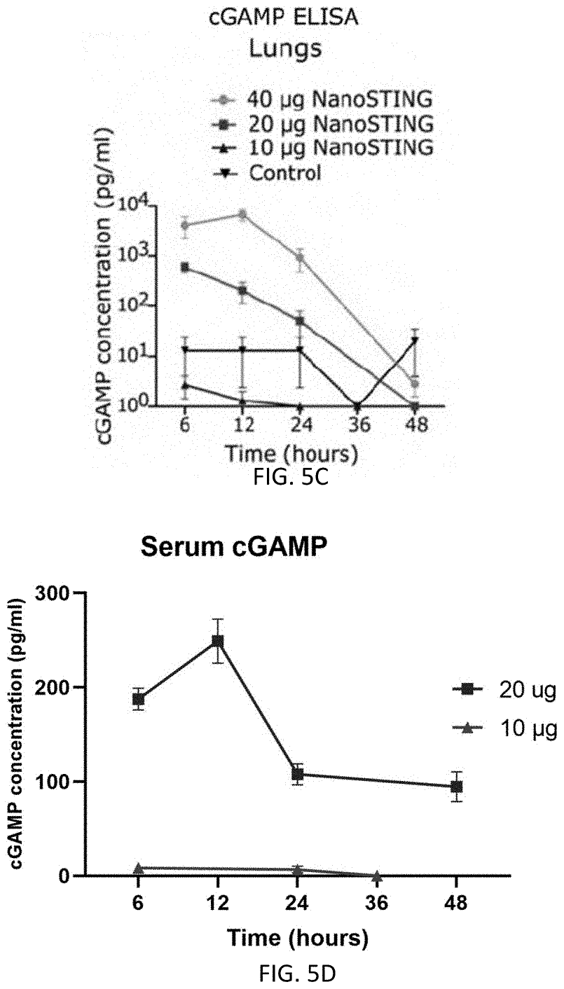

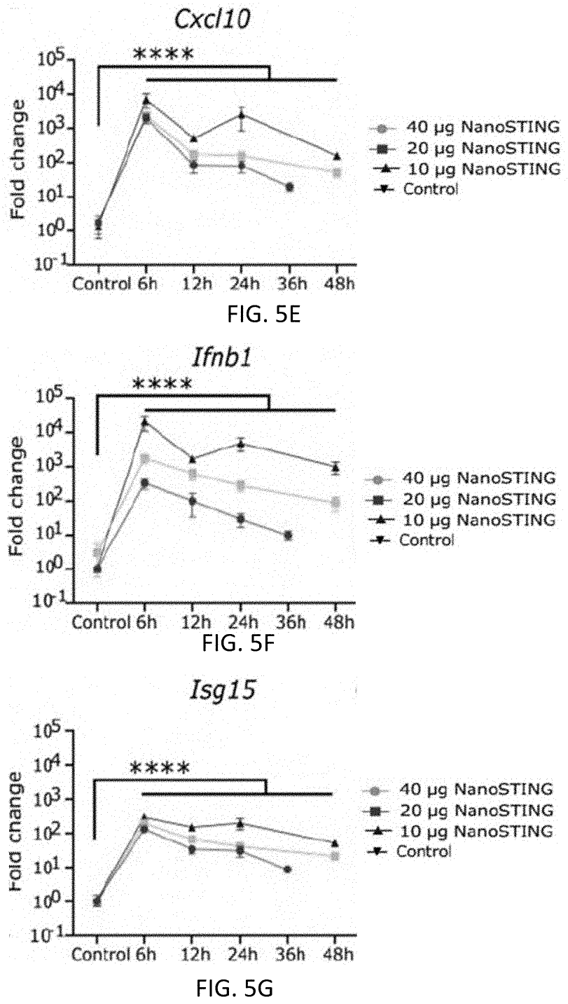

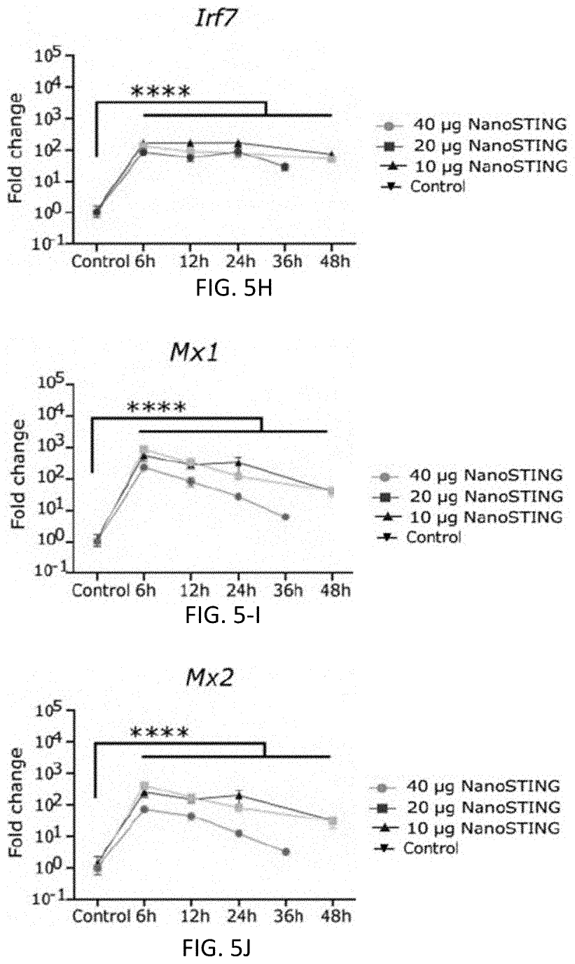

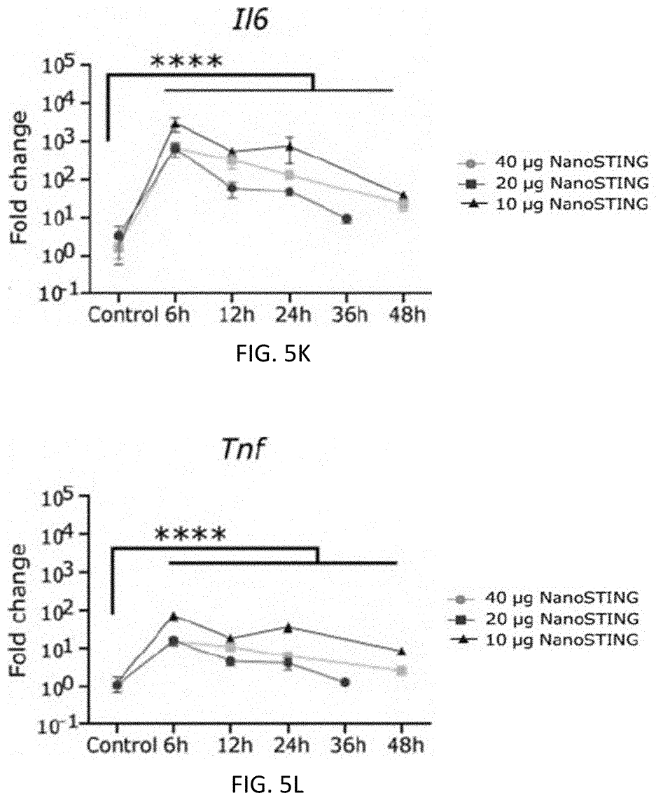

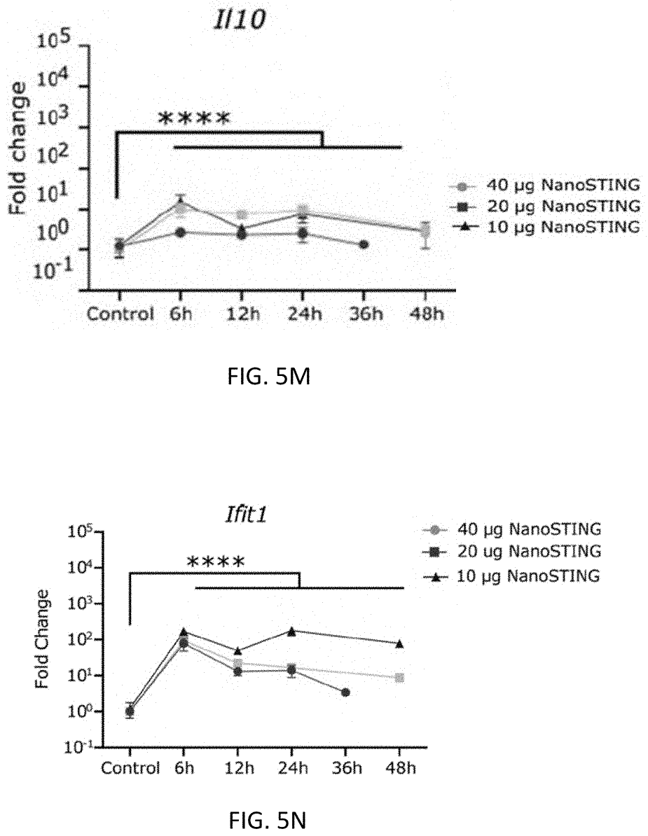

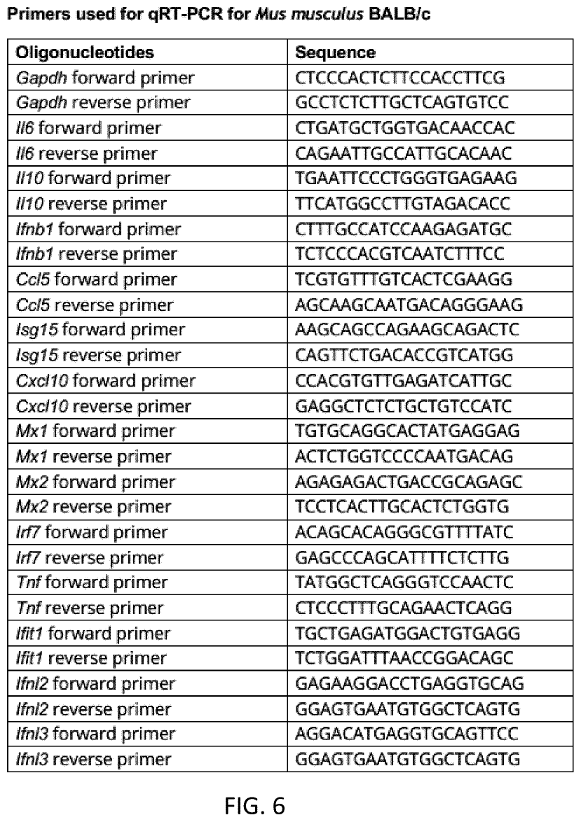

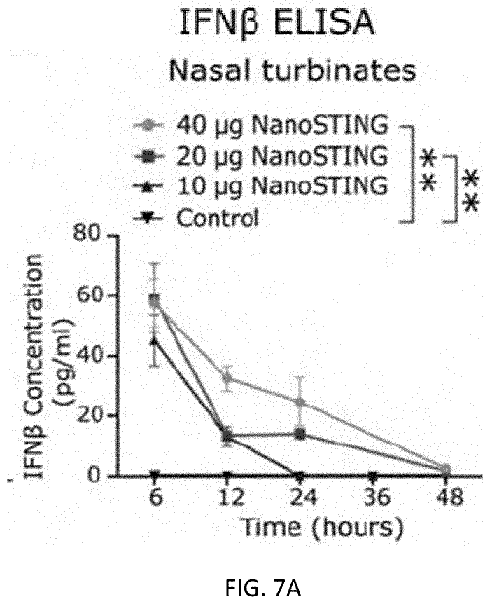

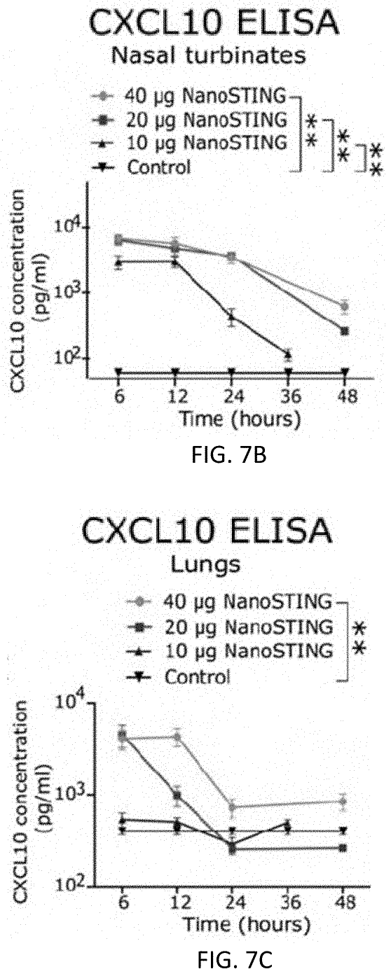

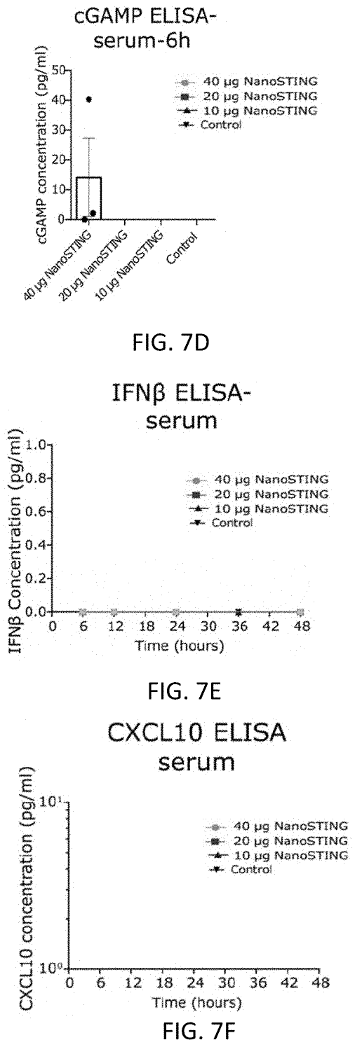

Although cGAMP is a potent natural activator of STING and therefore acts as an immunotransmitter, its clinical utility is hampered by lack of cellular penetration and rapid degradation by plasma ectonucleotide pyrophosphatase phosphodiesterase 1 (ENPP1), leading to an in vivo half-life of only ˜35 min. Varying amounts of NanoSTING (10-40 μg) were delivered intranasally to groups of BALB/c mice. Next, the nasal turbinates and lungs were harvested and cGAMP levels were assayed using quantitative ELISA (FIGS. 5A and 5B). A dose-dependent increase in the concentration of GAMP in the nasal turbinates was observed; at the low dose (10 μg), cGAMP concentrations were quantified up to 12 h with a return to baseline at 24 h, whereas at the higher doses (20-40 μg), cGAMP concentrations were quantified for 24 h with a return to baseline at 48 h (FIG. 5B). In the lungs, cGAMP was only detectable at the higher concentrations (20 and 40 μg) (FIG. 5C). In the same animals, the sera was profiled and cGAMP was not detected at any timepoints in circulation, even at the highest dose (40 μg) (FIGS. 7D, 7E, and 7F). These data confirmed that NanoSTING can transport cGAMP to the cells of the nasal passage in a concentration and time-dependent manner without systemic exposure.

The biological implications of NanoSTING's ability to deliver cGAMP and activate the STING pathway were evaluated using a panel of 10 genes to comprehensively measure the immune response. The panel comprised of the effector cytokines, C—X—C motif chemokine ligand 10 (Cxcl10) and interferon beta (Ifnb); Interferon stimulated genes (ISG) including Isg15, interferon regulatory factor 7 (Irf7), myxovirus resistance proteins 1 & 2 (Mx1 and Mx2), and interferon-induced protein with tetratricopeptide repeats 1 (Ifit1); and non-specific pro-inflammatory cytokines (Il6, Il10, and Tnf). BALB/c mice received varying doses of intranasal NanoSTING, and quantitative qRT-PCR was performed on the nasal turbinates (6-48 h) (FIG. 5A). The effector cytokines Cxcl10 and Ifnb showed maximal induction (7,000 to 20,000-fold induction) that remained elevated at 48 h (FIGS. 5D and 5E). The five ISGs demonstrated strong induction from 6 h (300 to 1,000-fold) to 24 h, followed by decay from 24-48 h (FIGS. 5F, 5G, 5H, 5-I, 5J, and 5N). NanoSTING's inflammatory response was linked to the IFN pathway as the pro-inflammatory cytokine Il6 showed brief induction at 6 h (5,000-fold), decayed significantly by 24 h, and was at baseline at 48 h (FIG. 5K). Furthermore, Tnf and Il10 showed only weak induction (15 to 60-fold) (FIGS. 5L and 5M). These results demonstrated that NanoSTING elicits a rapid and sustained inflammatory response triggering both effector cytokines and ISGs but only minimal activation of non-specific pro-inflammatory cytokines.

Since the qRT-PCR data suggested strong induction of the effector cytokines, Ifnb and Cxcl10, the concentrations of IFN-β and CXCL10 proteins in the nasal turbinates were quantified. Consistent with the transcriptional data, quantitative ELISA confirmed that both IFNβ and CXCL10 could be detected in the nasal turbinates and lungs for up to 24 h (FIGS. 7A, 7B, and 7C). IFN-β and CXL10 were not observed in serum tested from the same animals (FIGS. 7D, 7E, and 7F), demonstrating that stimulation of innate immunity by intranasal NanoSTING was localized and not systemic.

### Example 3: Identification of Robust IFN-I Signature in the Lungs of Hamsters Following NanoSTING Administration

This example describes experimental confirmation of the induction interferon-dependent and interferon-independent pathways following intranasal administration of NanoSTING in hamsters.

The hamster is a well-characterized model for the SARS-CoV-2 challenge and mimics severe disease in humans; animals demonstrate easily quantifiable clinical disease characterized by rapid weight loss, very high viral loads in the lungs, and extensive lung pathology. Additionally, unlike the K18-hACE2 transgenic model, hamsters recover from the disease (like most humans) and hence offer the opportunity to study the impact of treatments both in the lungs (disease) and nasal passage (transmission).

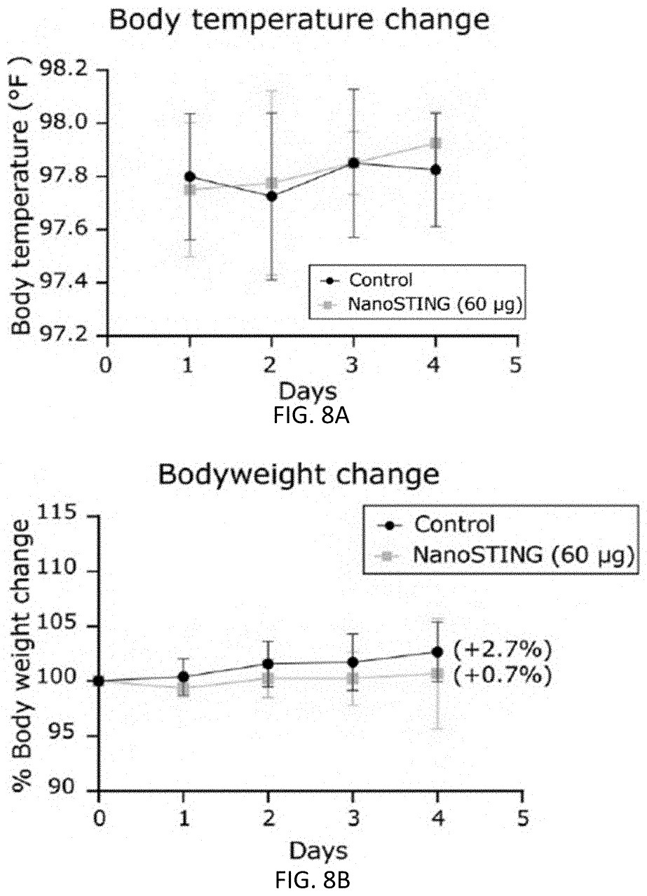

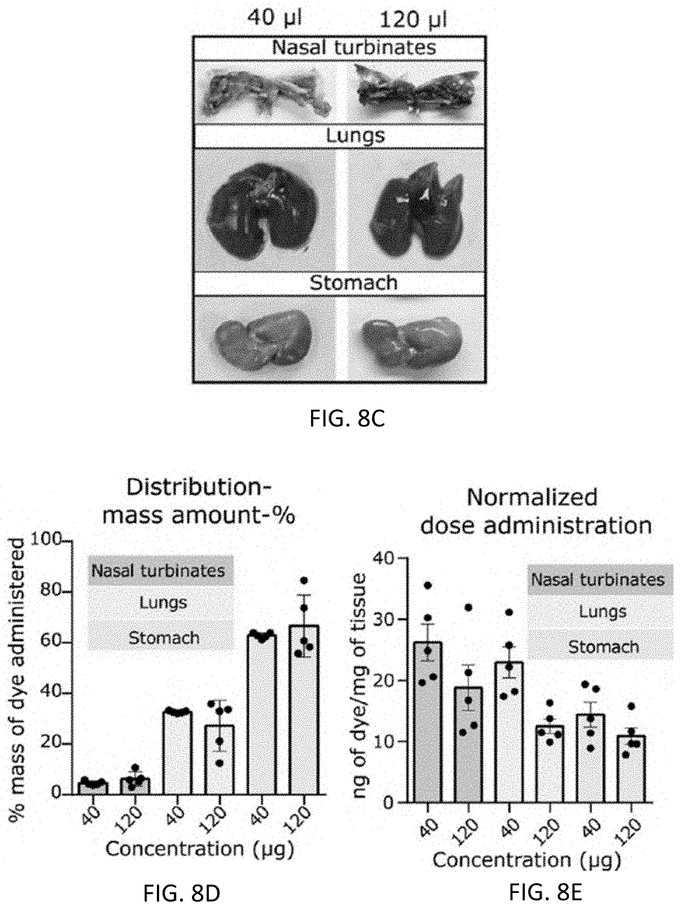

Biodistribution was examined by altering the transport volume of intranasally delivered NanoSTING. Intranasal administration of Evan's blue dye in low and high volumes (40 μl and 120 μl) resulted in staining of the nasal turbinates, lung, and stomach in hamsters (FIGS. 8C, 8D, and 8E). However, at both volumes, there was a significant amount of the dye delivered to the nasal turbinates and lung (intended target organs) (FIG. 8C), and the normalized ratio of distribution to these tissues was independent of the volume of administration (FIGS. 8D and 8E). These results suggested that biodistribution after intranasal delivery of liquid formulations was not impacted by the volume of inoculum in the hamster model.

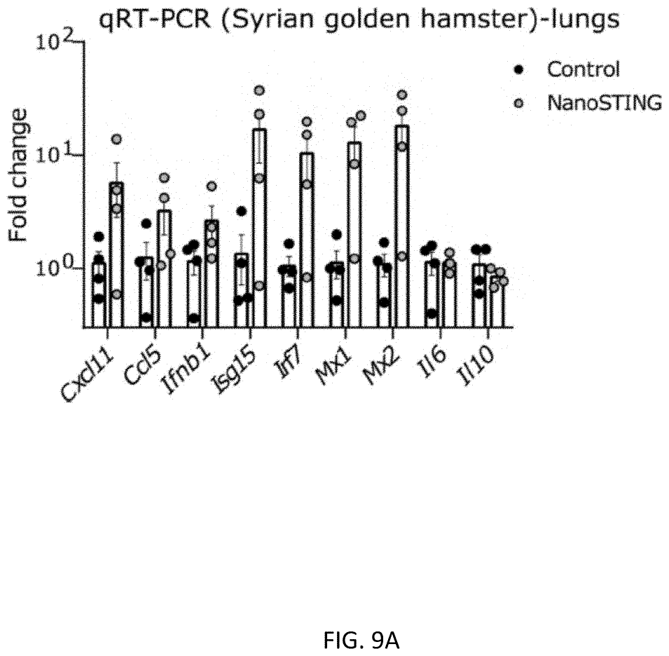

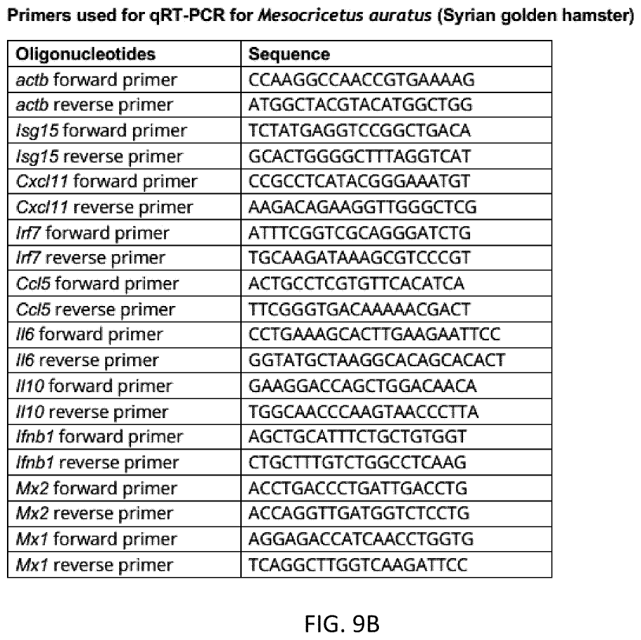

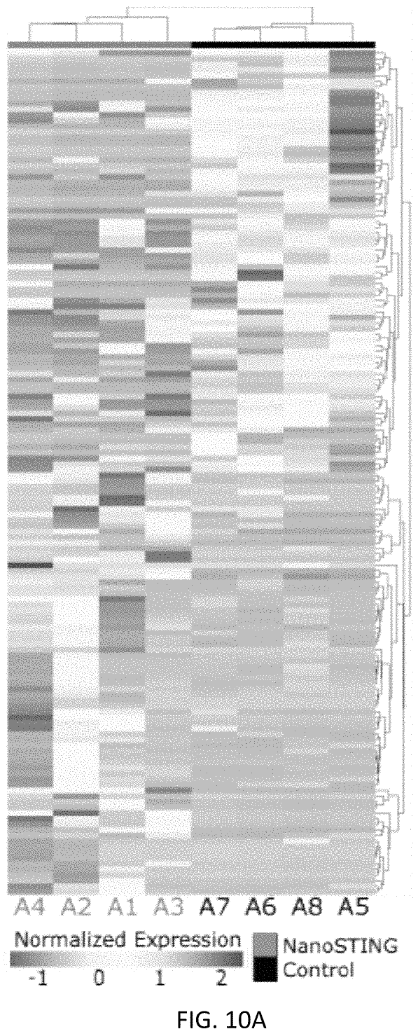

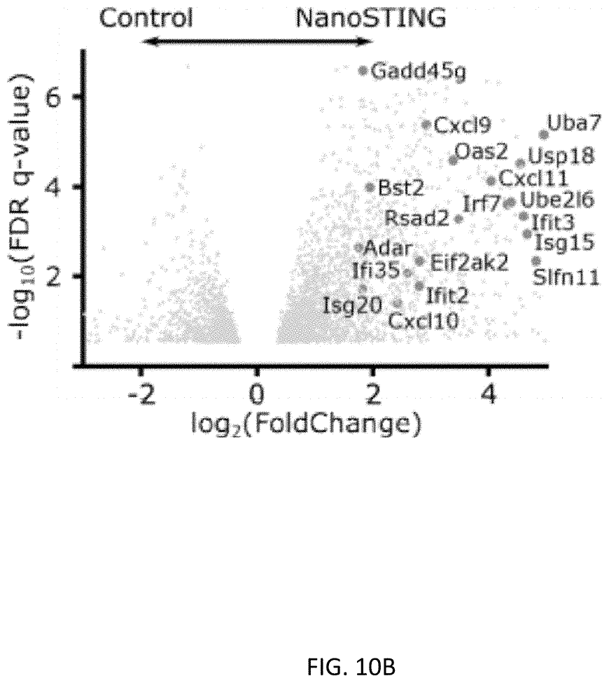

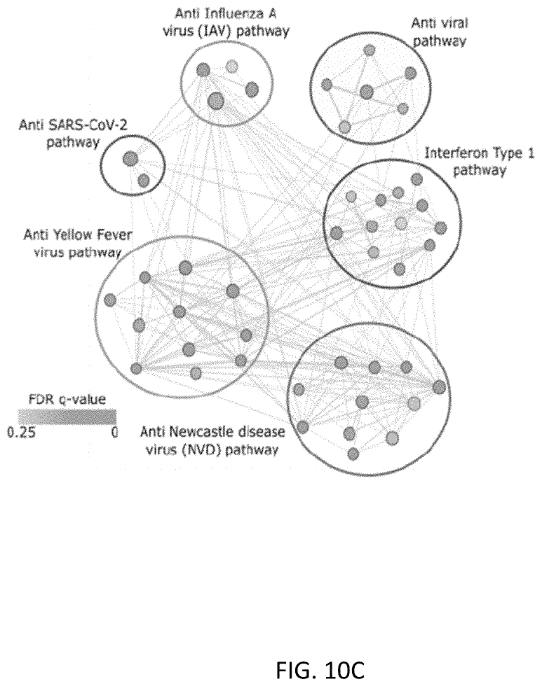

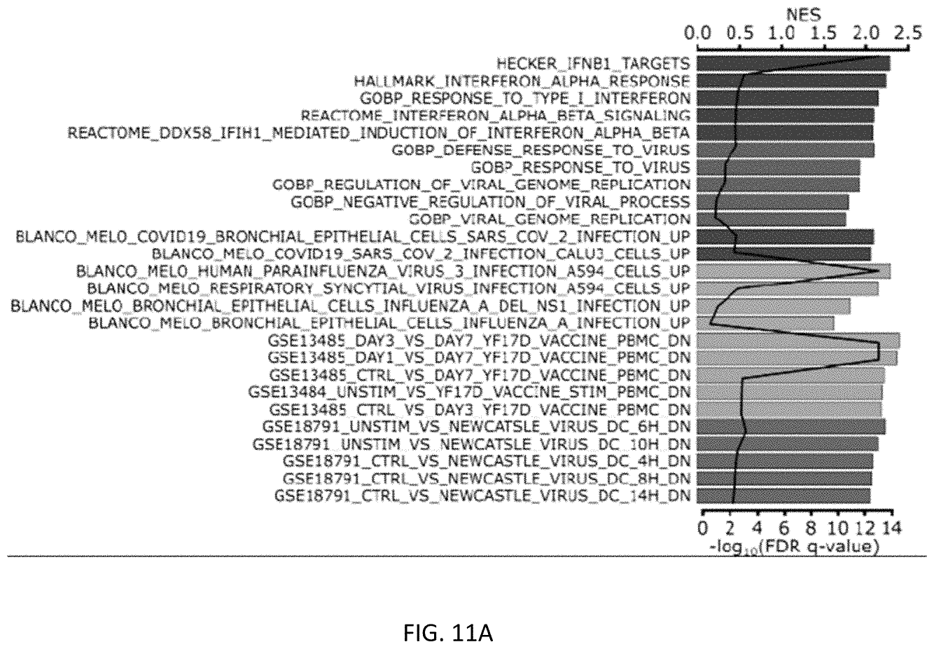

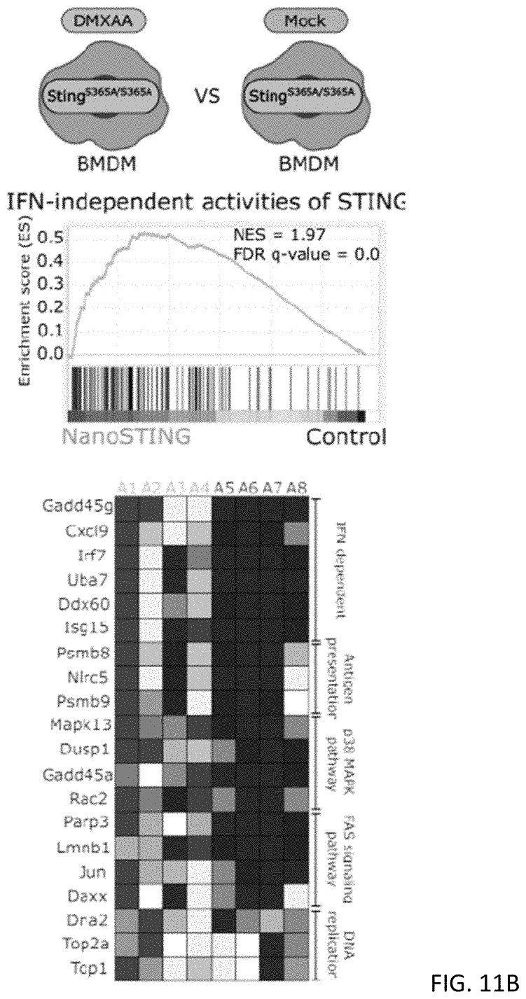

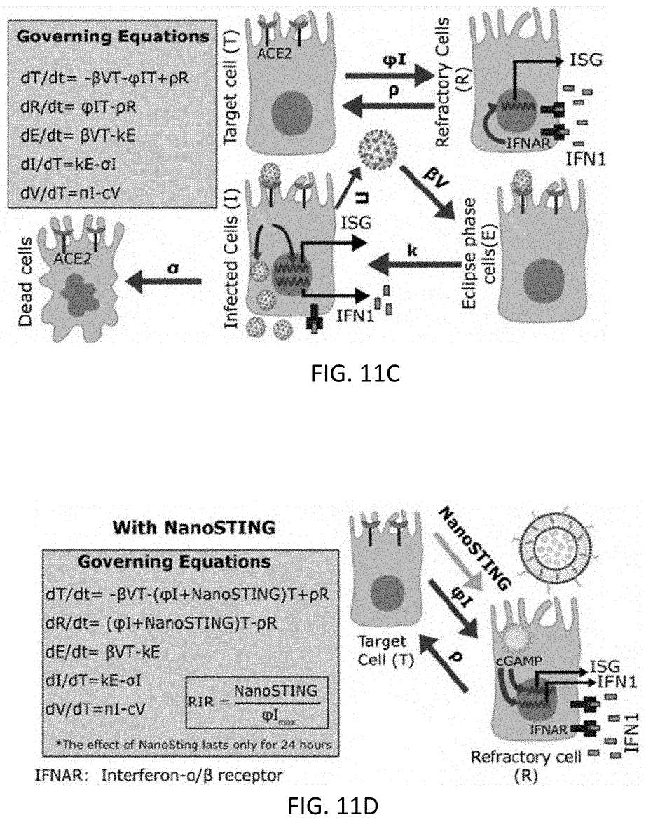

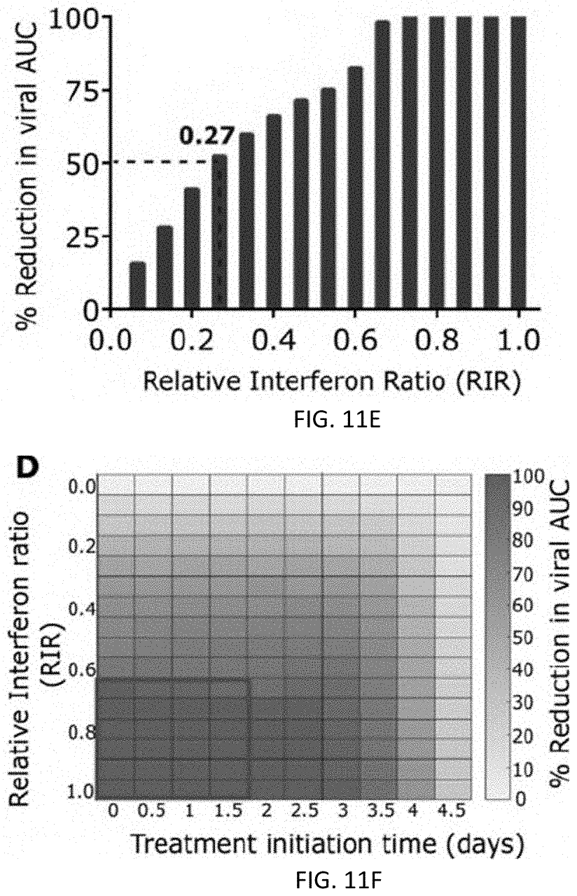

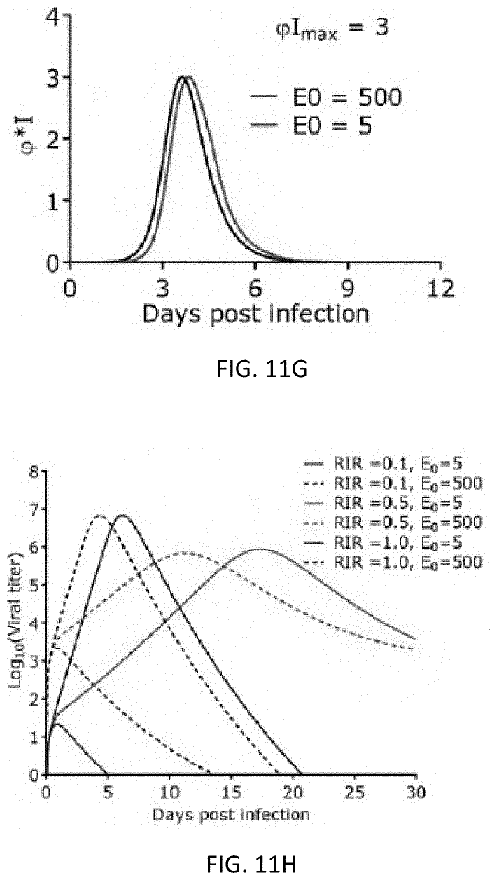

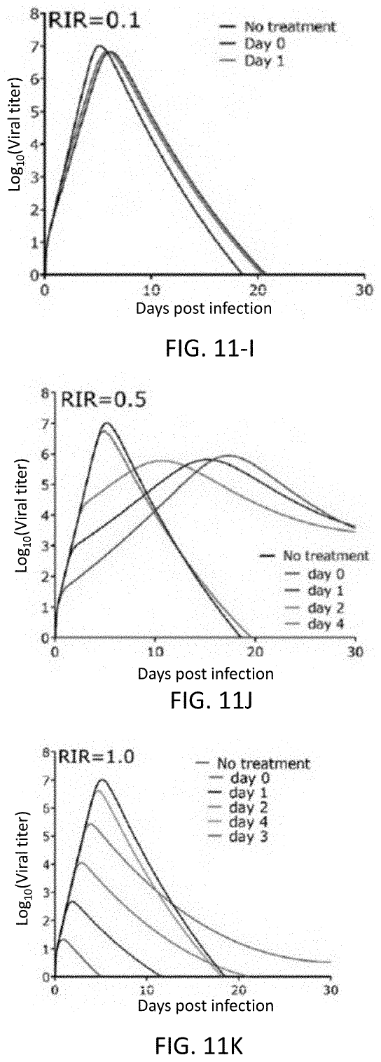

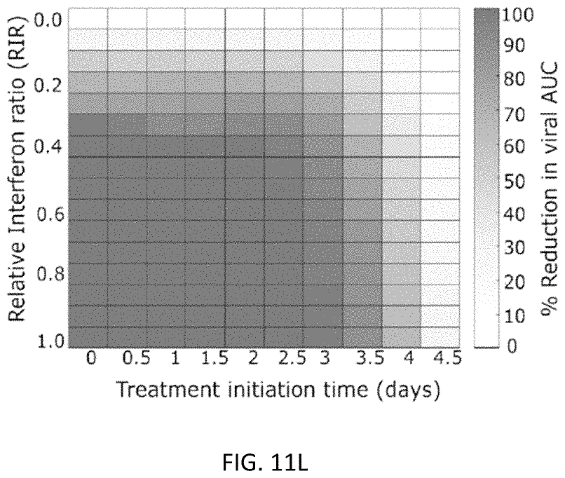

To assess the impact of intranasal NanoSTING on the lungs, groups of hamsters received daily doses of either NanoSTING (60 μg) or PBS (control) for four consecutive days. Both groups of animals showed no differences in clinical signs, such as temperature or bodyweight between the two groups (FIGS. 8A and 8B). On day 5, the lungs from the hamsters were isolated for unbiased whole-transcriptome profiling using RNA-sequencing (RNA-seq). At a false-discovery rate (FDR q-value <0.25), a total of 2,922 differentially expressed genes (DEGs) were identified between the two groups (FIG. 10A). A type I IFN response was induced in NanoSTING treated lungs, comprising canonical ISGs, including Mx1, Isg15, Uba7, Ifit2, Ifit3, Ifit35, Irf7, Adar, and Oas2 (FIG. 10B). The effector cytokines, Cxcl9-11 and Ifnb, were also induced in treated hamsters (FIG. 10C) and showed robust induction of direct antiviral proteins, such as Ddx60 and Gadd45g (FIG. 11A). Next, a gene-set enrichment analysis was performed to compare the differentially induced pathways upon treatment with NanoSTING. Changes in these populations were examined against the Molecular Signatures Database (Hallmark, C2, and C7 curated gene sets). A distinct cluster of pathways related to both type I and type III interferons in the lungs of NanoSTING treated animals was observed. The specificity of the response was confirmed by qRT-PCR analyses by quantifying Mx1-2, Isg15, Irf7, Cxcl11, Ifnb, Il6, and Il10 (FIG. 9A). Since the gene signature of interferon-independent activities of STING is known, geneset enrichment analyses (GSEA) were performed and confirmed that NanoSTING activated interferon-independent pathways and associated proteins (FIGS. 11A and 11B).

Collectively, these results demonstrated that cGAMP mediated activation of STING by NanoSTING efficiently engaged both interferon-dependent and interferon-independent antiviral pathways in the lung.

### Example 4: Prediction of the Effects of Early Treatment NanoSTING on Viral Replication Using Quantitative Modeling

This example describes the results of modeling experiments performed to analyze the level of SARS-CoV-2 replication following administration of NanoSTING.

The in vivo mechanistic experiments demonstrated that NanoSTING induced a broad antiviral program by engaging the innate immune system. A mathematical model approach in combination with human viral load data was used to identify the treatment window and quantify the relative amount of type I IFN (or related pathways) elicited by NanoSTING required for therapeutic benefit. To simplify the framework of the model, tests were performed under the assumption that in vivo cGAMP only works to stimulate interferon responses. With this assumption, the range of relative interferon ratios (RIR, 0-1) needed to elicit via NanoSTING in comparison to the population level peak interferon responses observed upon SARS-CoV-2 infection was modeled (FIGS. 11C and 11D), and the influence on viral elimination was examined. Based on the model, an RIR of just 0.27 (27% of natural infection) was deemed sufficient to achieve a 50% reduction in viral load (based on the area under the curve, AUC), and RIR values of at least 0.67 will achieve 100% reduction in viral loads (FIG. 11E). Next, the window of initiation of treatment was modeled which revealed that intervention was most effective when initiated within 2 days after infection (FIG. 11F). By contrast, if the treatment was initiated after the peak of viral replication, even with an RIR of 1, improvement in outcomes cannot be readily realized (FIGS. 11F, 11G, 11H, 11-I, 11J, and 11K).

Collectively, these results from quantitative modeling predicted that: (a) a single dose of NanoSTING was needed to elicit only a moderate amount of IFN and this was likely within reach since our data supports large induction of IFNβ (FIGS. 5E, 5M, and 11B) and since natural infection with viruses like SARS-CoV-2 and Influenza A is known to suppress interferon production, and (b) the optimal treatment window was both as prophylaxis and soon after infection.

### Example 5: Treatment with NanoSTING Protects Against the SARS-CoV-2 Delta Strain and Induces Protection Against SARS-CoV-2 Reinfection

This example describes the level of protection from the SARS-CoV-2 Delta (B.1.617.2) strain following a single dose of NanoSTING in hamsters.

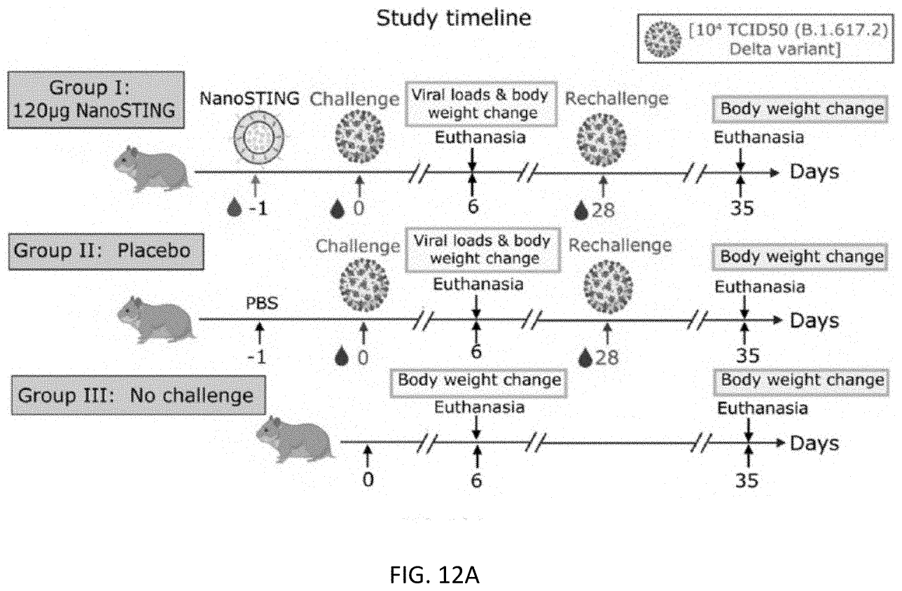

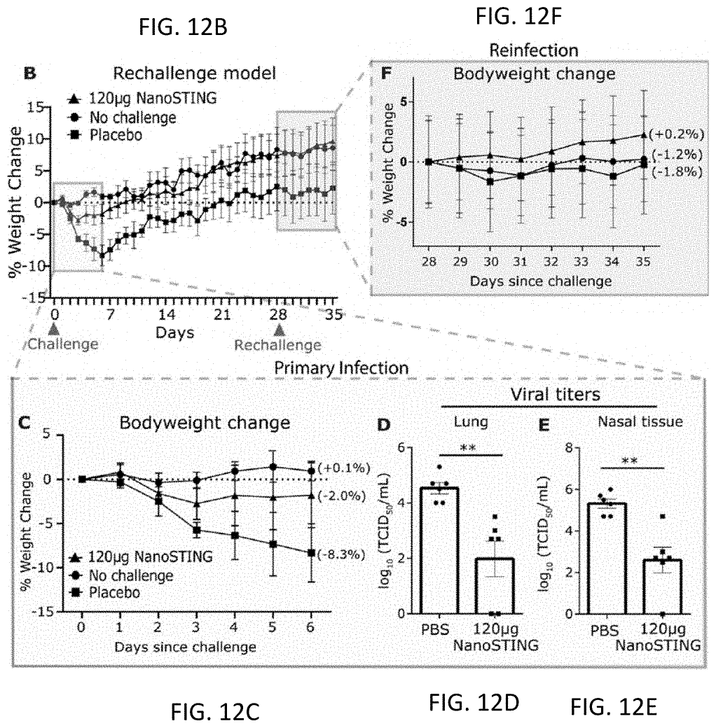

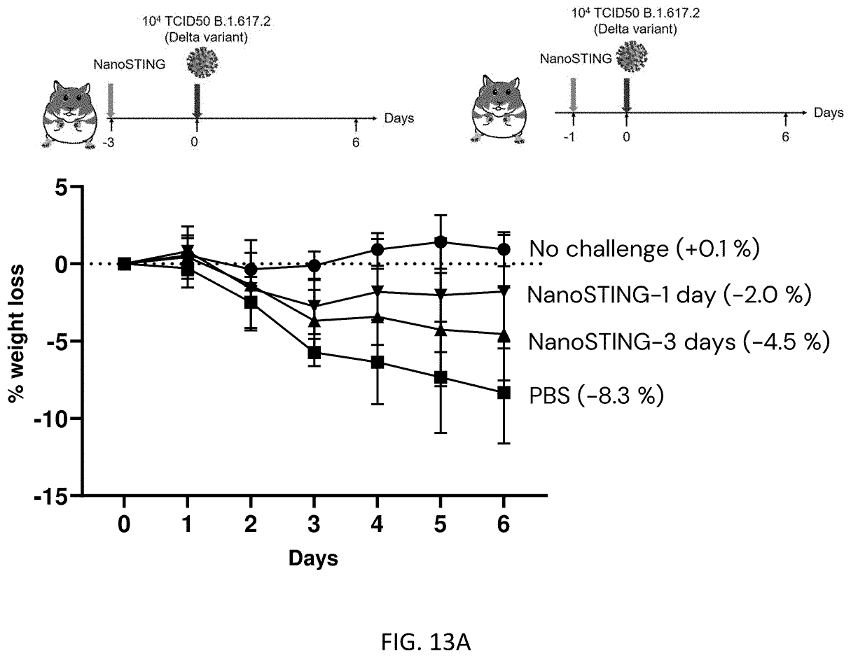

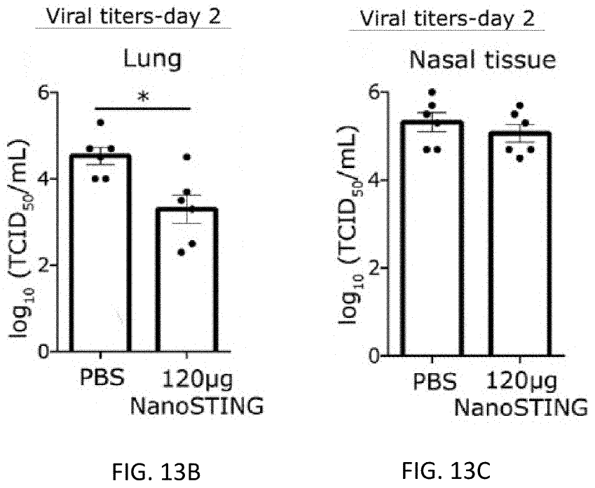

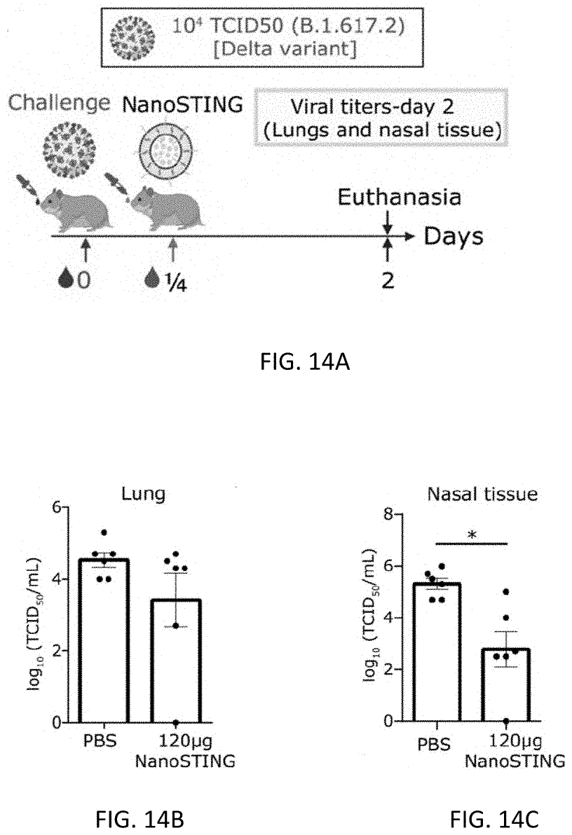

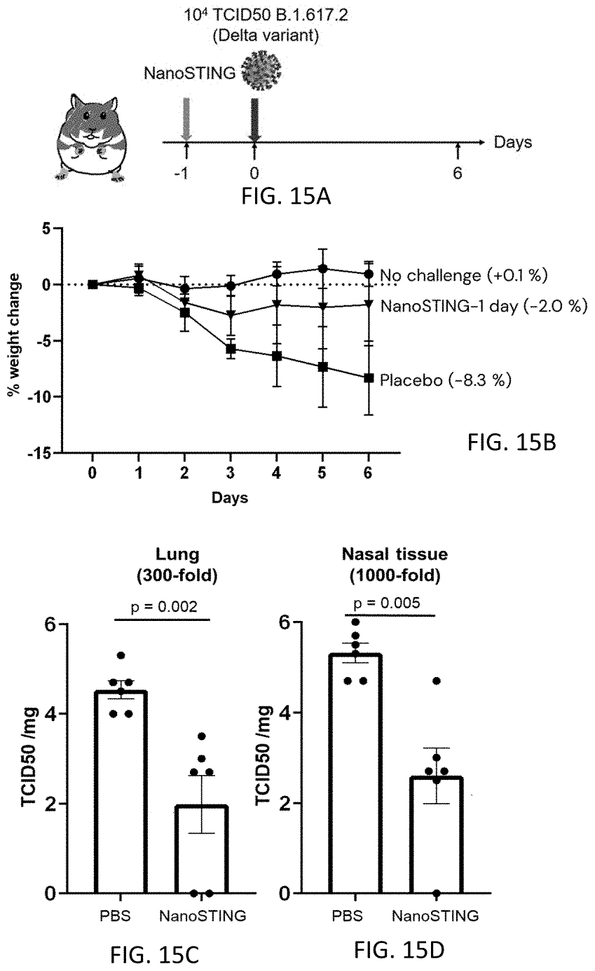

The Delta strain (B.1.617.2) was chosen because it causes both upper and lower tract disease and has increased disease severity compared with prior variants (Wuhan and Beta strains). Groups of 12 animals were treated with a single intranasal dose of 120 μg NanoSTING and then infected 24 h later with 10⁴ 50% tissue culture infectious dose (TCID50) of the Delta variant through the intranasal route (FIG. 12A). Animals in the placebo-treated (PBS) group exhibited severe weight loss, with a mean peak weight loss of 8.3%. By contrast, animals treated with NanoSTING were largely protected from weight loss (mean peak weight loss of only 2.0%) (FIG. 12C). This small amount of loss in weight was similar to the results obtained by adenoviral vectored vaccines challenged with either the Wuhan or Beta strains. Half of the animals were sacrificed at day 2 (peak of viral replication) to quantify the infectious viral loads. Even with the highly infectious Delta strain, NanoSTING reduced infectious viral loads in the lung post 2 days of infection by 300-fold compared to placebo-treated animals (FIG. 12D). This reduction in viral loads in the lung closely tracked with prevention of weight loss in these animals. Quantification of the viral loads in the nasal compartment revealed that NanoSTING treatment reduced infectious viral loads in the nasal compartment post 2 days of infection by 1,000-fold compared to placebo-treated animals (FIG. 12D). The reduction in viral replication in the nasal compartment modeled propensity of human transmission and confirmed that treatment with NanoSTING decreased the likelihood of transmission. In order to map the duration of efficacy of prophylactic NanoSTING treatment, hamsters were next administered a single intranasal dose of NanoSTING and subsequently challenged 72 h later with 10⁴ TCID50 of the Delta strain (FIG. 13A). Even when administered at 72 h before exposure, NanoSTING showed moderate protection from weight loss and a significant reduction in infectious viral loads (FIGS. 13A, 13B, and 13C). To test the effects of NanoSTING following viral, intranasal NanoSTING was delivered 6 h after exposure to the Delta virus (FIG. 14A). There was a 340-fold and 13-fold reduction in infectious virus in the nasal passage and lung, respectively (FIGS. 14B and 14C).

These results demonstrated that a single dose treatment with NanoSTING effectively minimized clinical symptoms, protected the lung, and reduced infectious viruses in the nasal passage.

One of the advantages of engaging the innate immune system to clear viral infection is that this process mimics natural infection while minimizing the danger of clinical symptoms. To test if adaptive immunity is engaged, hamsters were intranasally administered with NanoSTING, and 24 h later, the animals were challenged with 10⁴ TCID50 of the Delta variant (FIG. 12A). Administration of NanoSTING reduced weight loss during the primary challenge. Unlike the placebo-treated animals, the animals treated with NanoSTING regained their weight such that by day 4 their weights were not significantly different from animals that had not been challenged. The mitigation in weight loss was attributed to the reduction in infectious viral loads in lung and nasal tissue (FIGS. 12C, 12D, and 12E). On day 28, animals were rechallenged with the Delta variant. NanoSTING treated animals were completely protected from weight loss during the secondary challenge, and the animals' weight was identical to animals that were not previously challenged (FIG. 12F).

These results established that a single intranasal treatment with NanoSTING activated the antiviral program of innate immunity and defended against clinical disease during primary infection while facilitating adaptive immunity that offered durable protection from reinfection.

### Example 6: Effects of NanoSTING Treatment Against IFN Evasive SARS-CoV-2 Alpha Variant (B.1.1.7)

This example examines the effects of varying doses of NanoSTING and varied doses of treatment against the SARS-CoV-2 Alpha strain.

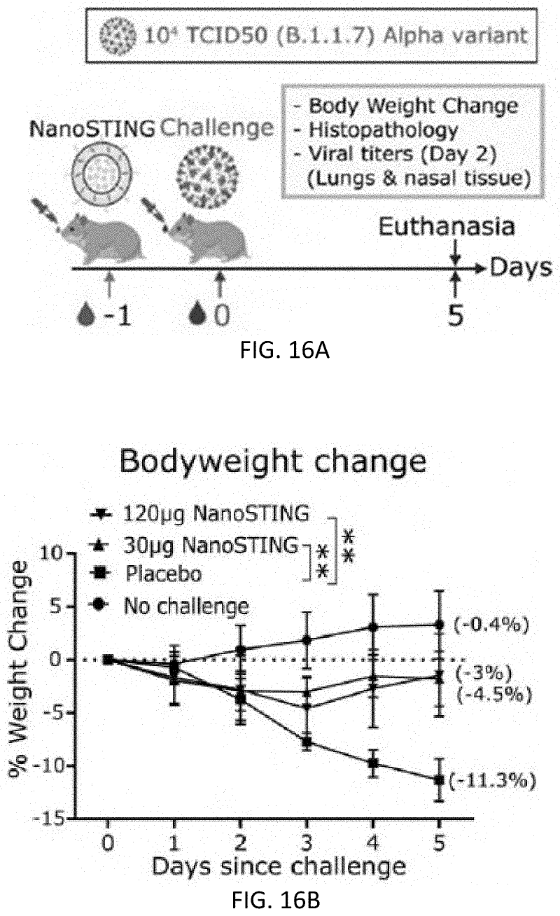

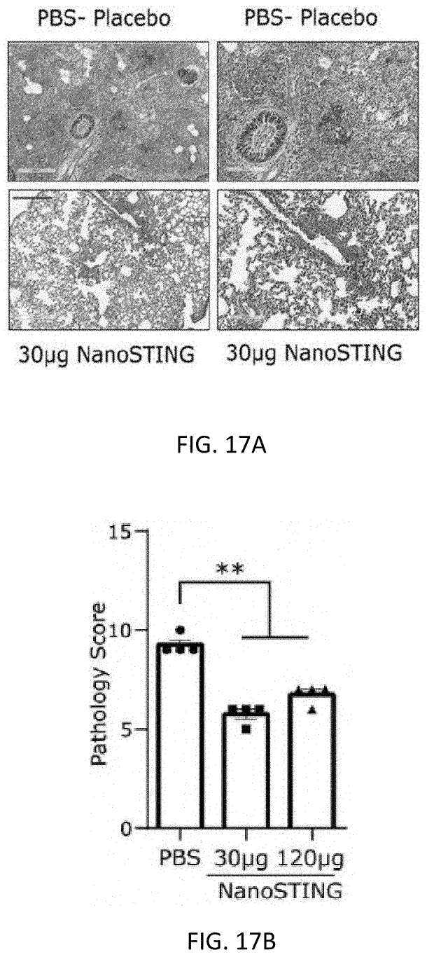

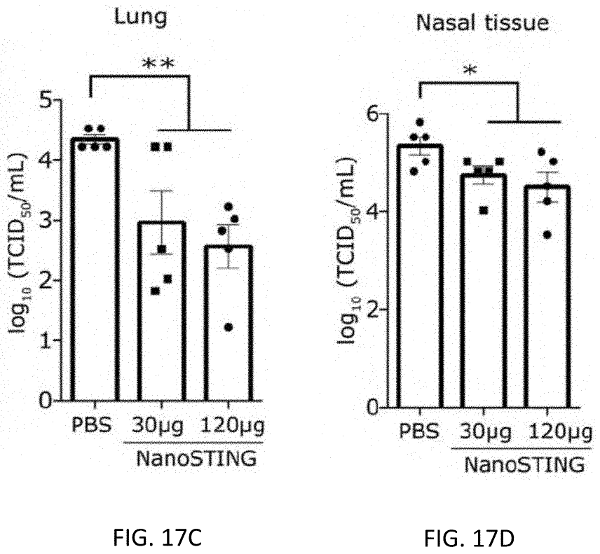

The Alpha (B.1.1.7) variant is known to be resistant to IFN-1 signaling in vitro and thus provides a challenging model to test the efficacy of NanoSTING. Hamsters were pre-treated with two intranasal doses of NanoSTING (30 μg and 120 μg) and 24 h later challenged with 10⁴ TCID50 of the Alpha variant (FIG. 16A). Treatment with either dose of NanoSTING protected the hamsters from severe weight loss (FIG. 16B). An integrated scoring rubric (range from 1-12) was used to account for both pathology and disease to analyze the lung tissue on Day 6 after the viral challenge. NanoSTING treated animals had significant reductions in aggregate pathology score with minimal evidence of invasion by inflammatory cells or alveolar damage (FIGS. 17A and 17B). In addition, the viral loads in the lung and nasal compartments were quantified and a significant reduction of viral loads in both compartments was found even by Day 2 (FIGS. 17C and 17D).

Thus, these results established that treatment with intranasal NanoSTING reduced in vivo replication of SARS-CoV-2 by orders of magnitude and conferred protection against IFN-I evasive strains of SARS-CoV-2.

### Example 7: Evaluation of Transmission of the SARS-CoV-2 Omicron Variant Following NanoSTING Treatment

This examples describes experimental confirmation that treatment with NanoSTING is capable of blocking transmission and preventing infection of the more infectious SARS-CoV-2 Omicron variant.

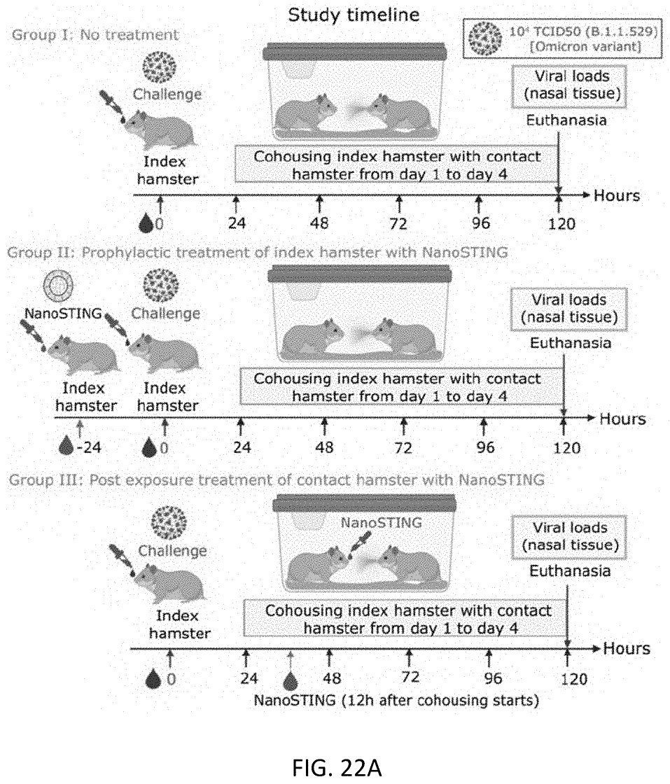

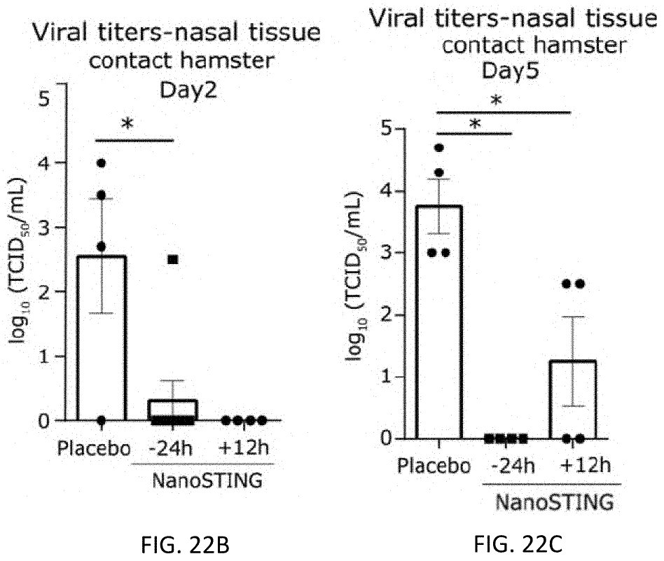

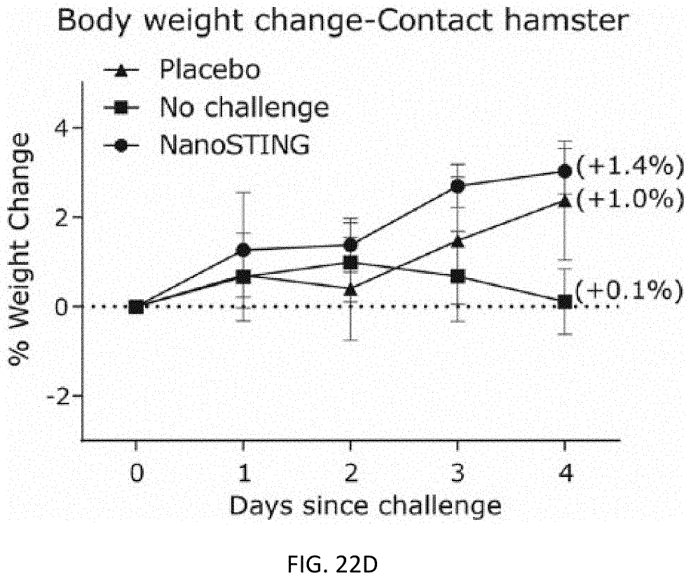

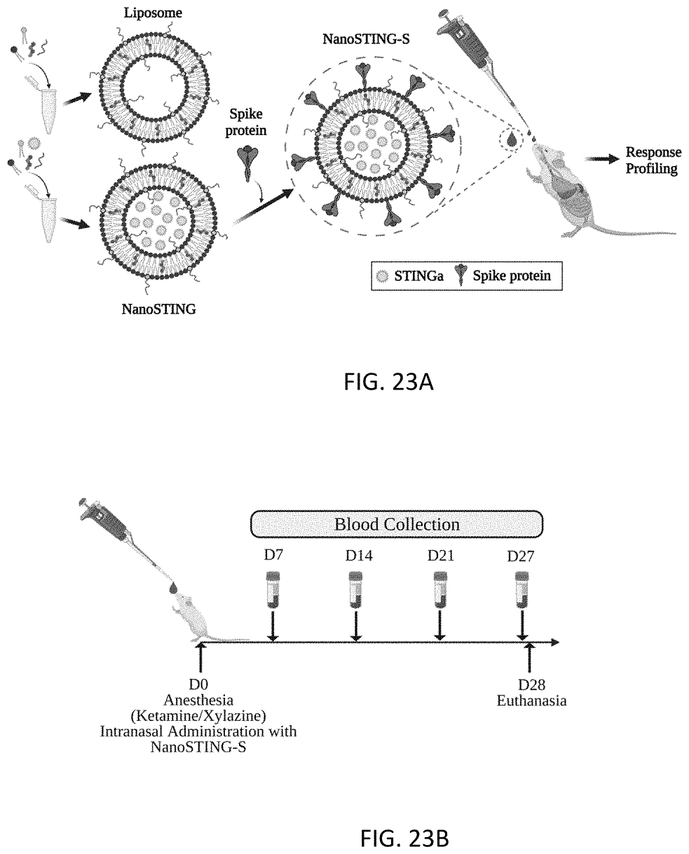

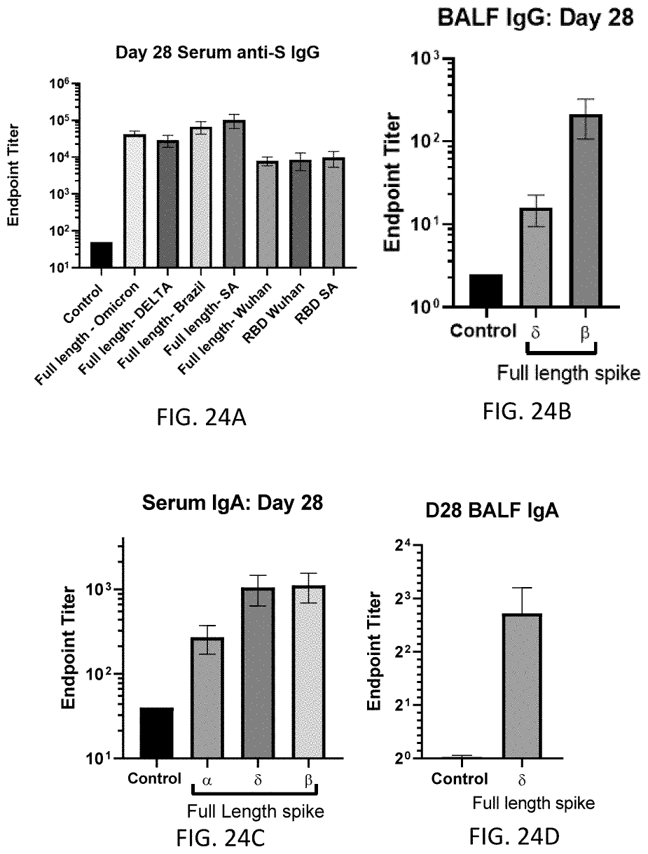

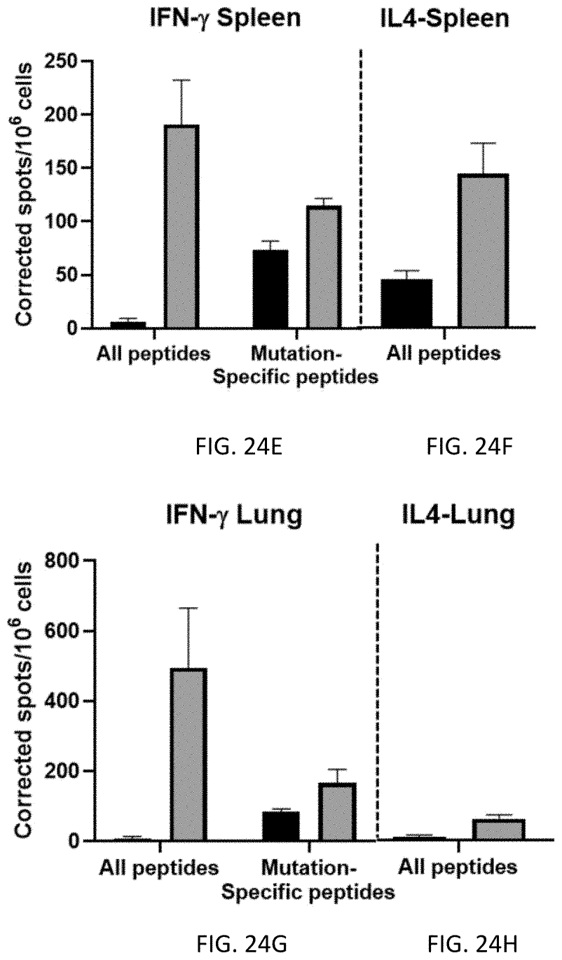

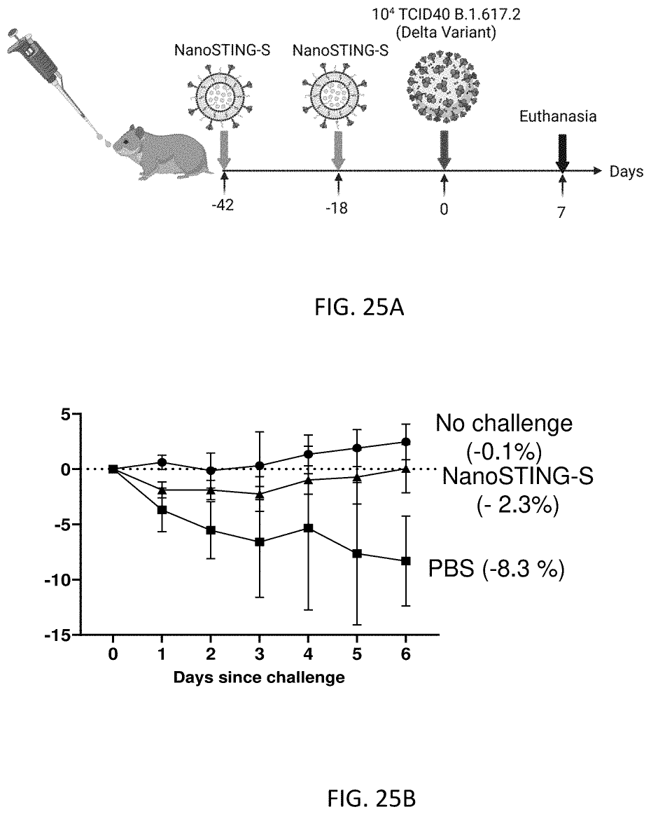

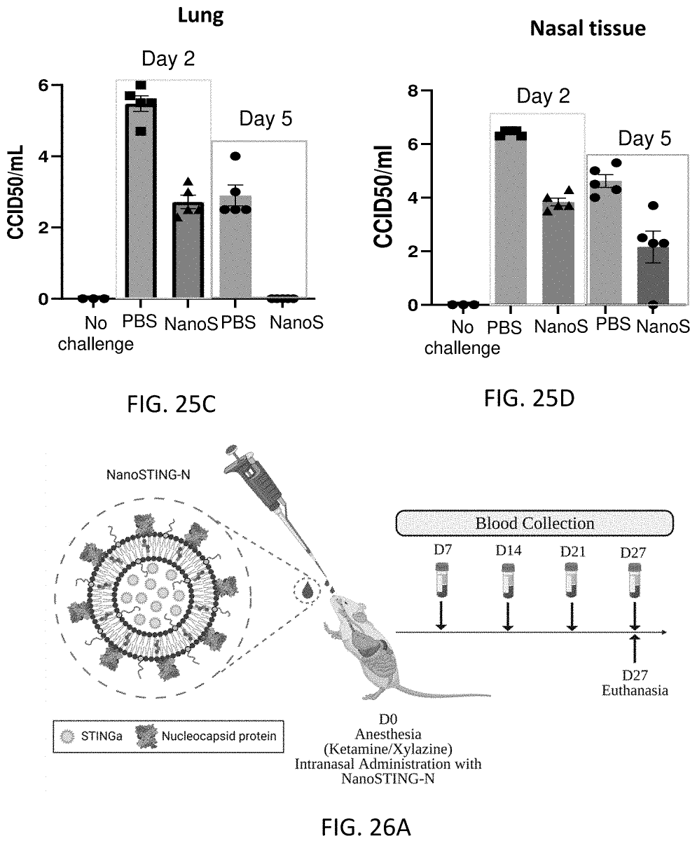

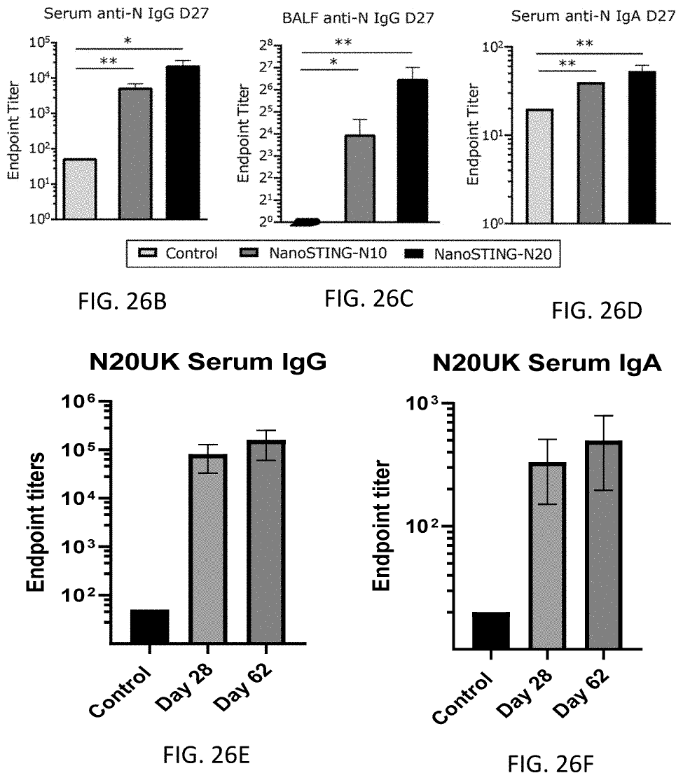

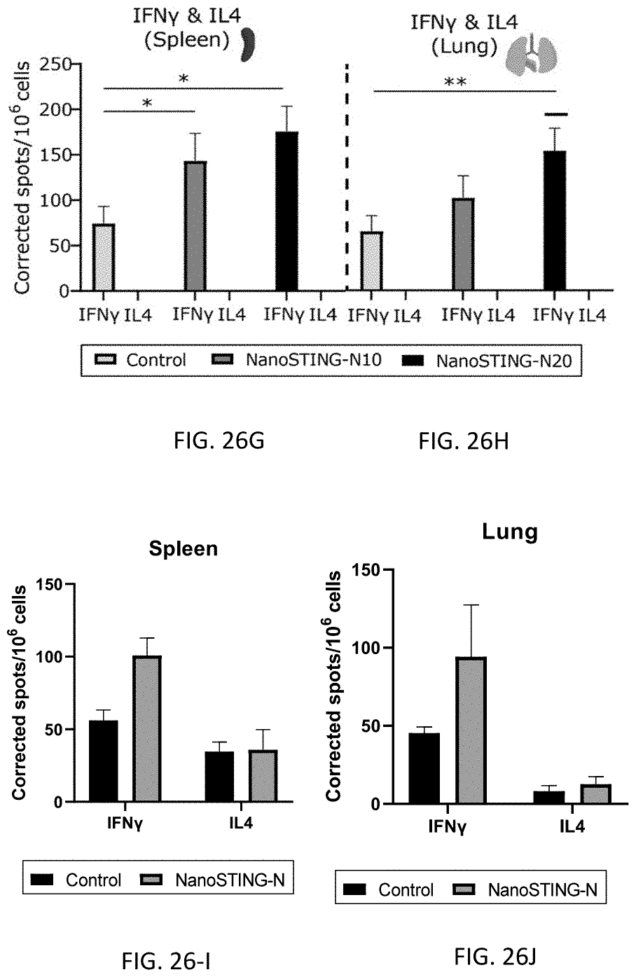

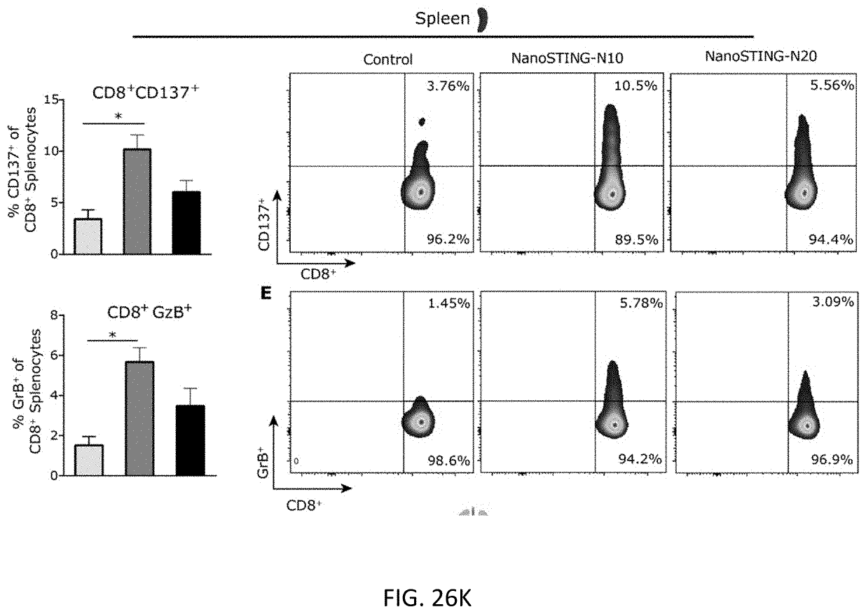

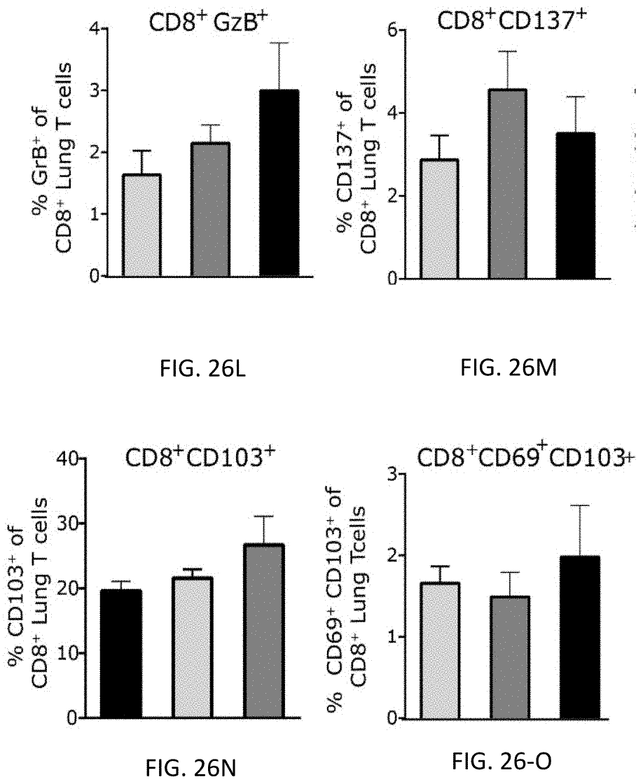

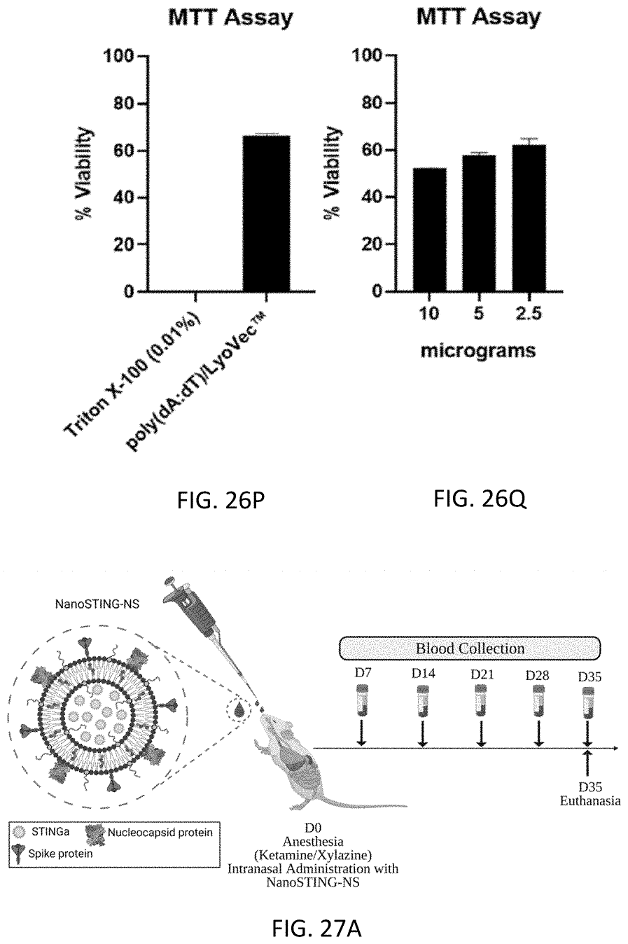

The Omicron variant (B.1.1.529) is the most infectious among the known strains of SARS-CoV-2. Using the Omicron variant sets up the highest bar for NanoSTING to prevent viral spread. To assess whether prophylactic treatment of infected hamsters prevents transmission to contact hamsters, and whether post-exposure treatment of contacted hamsters mitigates viral replication an experiment was conducted with three groups of 16 hamsters. In each group, eight hamsters were intranasally infected with the 104 TCID50 of the SARS-CoV-2 Omicron variant, and one day after infection, each hamster was paired with a cohoused sentinel hamster (FIG. 22A). None of the infected animals showed weight loss (FIG. 22D). Sentinel hamsters were either: (a) cohoused with NanoSTING (120 μg) treated index hamsters (group 2) or (b) treated with NanoSTING after cohousing with infected but untreated hamsters (group 3). As with the other strains of SARS-CoV-2 tested, NanoSTING pre-treatment of the infected hamsters blocked transmission in nearly all animals (7/8 animals treated were virus-free vs 1/8 untreated animals were virus-free). Significantly, post-exposure treatment of the sentinel hamsters was also effective at preventing infection (6/8 animals treated were virus-free), and all animals demonstrated a reduction in viral load (FIGS. 22B and 22C).

These results demonstrated that NanoSTING is highly effective at blocking transmission even with the highly infectious Omicron strain.

### Example 8: Effects of NanoSTING Treatment on Protection from Influenza Similar to Oseltamivir

This example identifies several experiments confirming that administration of NanoSTING is an effective treatment against influenza virus, as measured by weight loss, survival, and viral titer.

The influenza viruses have evolved multiple mechanisms to dampen the host's innate immunity, including the attenuation of interferon responses by the NS1 protein. One of the primary treatment options against Influenza involves post-exposure prophylaxis using Oseltamivir that inhibits the influenza neuraminidase protein. Thus, Oseltamivir (Tamiflu) can serve as a benchmark to evaluate the therapeutic impact of NanoSTING.

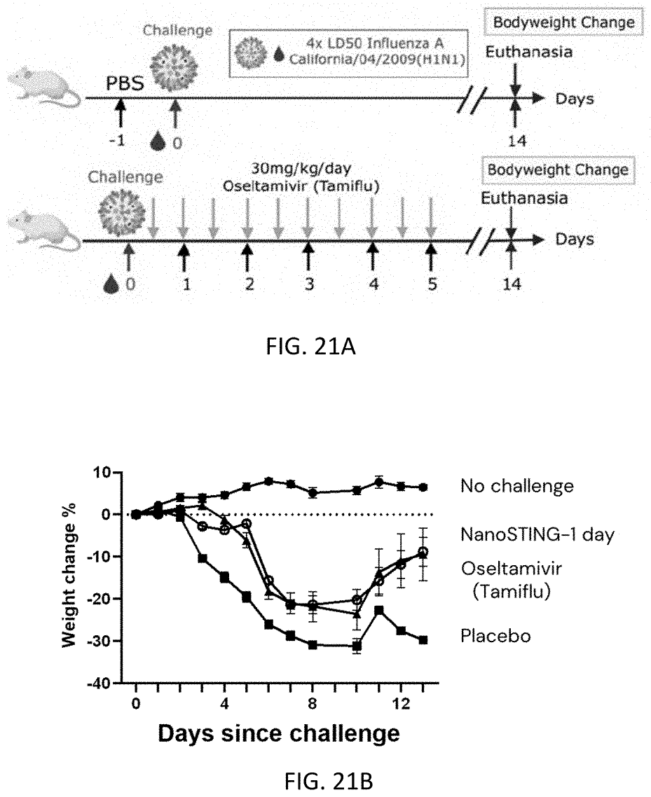

Groups of ten mice were treated with a single intranasal dose of NanoSTING and 24 h later challenged with 4×LD50 (lethal dose 50) sensitive strain of Influenza A/California/04/2009 (H1N1) (FIG. 21A). Animals were evaluated for 14 days and weight loss and survival as were used as the primary endpoints. One group of mice was treated with a clinically relevant dose of Oseltamivir (30 mg/kg/day), twice daily, for five days, as a positive control. The untreated animals started losing significant weight by day 3 (peak of viral infection), and by day 11, only one animal survived with a weight loss of 23% (FIG. 21B). On day 3, NanoSTING treated mice had no significant weight loss, similar to the unchallenged animals, and this was a significant improvement from even the Oseltamivir treated animals (FIG. 21B). The impact of the single dose of NanoSTING (40 μg) diminished after day 3. However, even accounting for this reduction, the mean peak weight loss and weight loss kinetics were not significantly different from Oseltamivir treated animals (FIGS. 21A and 21B). Taken together, these results illustrated that NanoSTING protected animals from influenza equivalent to clinically relevant doses of Oseltamivir.

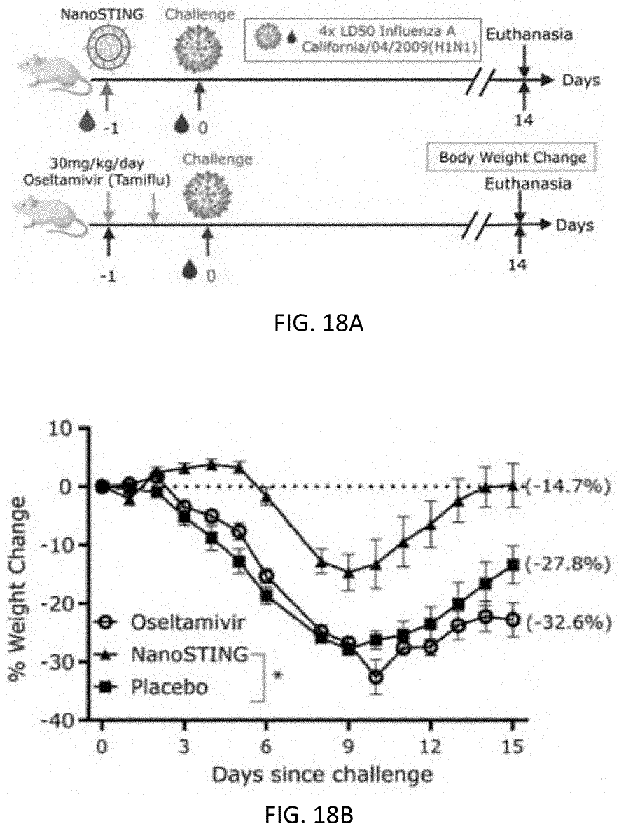

However, even accounting for this reduction, the mean peak weight loss, weight loss kinetics, and survival in treated animals were not significantly different from Oseltamivir treated animals (FIG. 18B). Animals were pre-treated with Osetamivir (30 mg/kg/day) or NanoSTING (40 μg) then challenged with 4×LD50 (lethal dose 50) sensitive strain of Influenza A/California/04/2009(H1N1) (FIG. 18A). NanoSTING protected the animals as evident from mean peak weight loss of −14%, −27% and −32% for NanoSTING, placebo, and oseltamivir treated animals respectively (FIG. 18B)

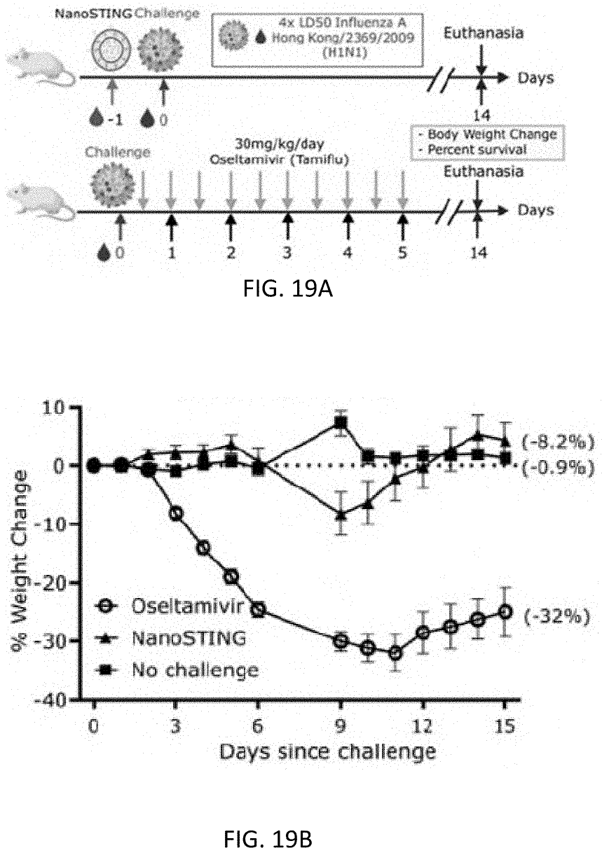

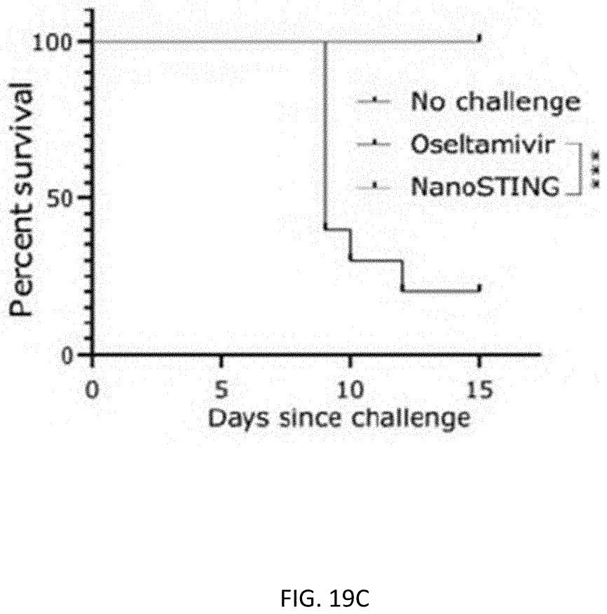

A single amino acid mutation (His275Tyr) with neuraminidase has led to oseltamivir-resistant influenza viruses in humans. Since NanoSTING relies on the host's innate immune response, an experiment was conducted to assess its potency against oseltamivir-resistant influenza A in mice. Groups of ten mice were treated with a single intranasal dose of NanoSTING (40 μg) and 24 h later challenged with 4×LD50 (lethal dose 50) resistant strain of Influenza A (FIG. 19A). The animals were evaluated for 14 days and weight loss and survival were used as the primary endpoints. One group of mice was treated with a clinically relevant dose of Oseltamivir (30 mg/kg/day), twice daily, for five days, as a positive control. Oseltamivir treated mice showed mean peak weight loss of −32% as compared to −8.2% for NanoSTING treated animals (FIG. 19B). The survival data for NanoSTING treated animals was significantly different from Oseltamivir treated animals (FIG. 19C). In aggregate, NanoSTING showed protective efficacy as compared to oseltamivir as evident from reduced weight loss and enhanced survival.

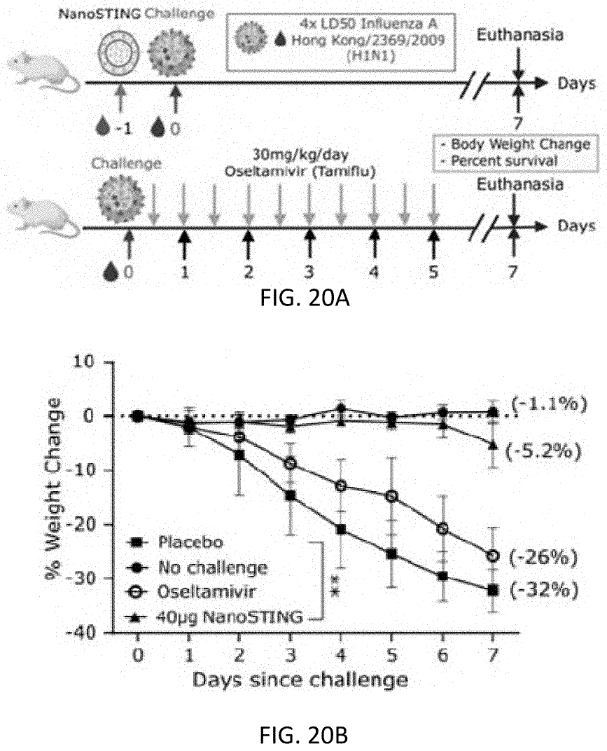

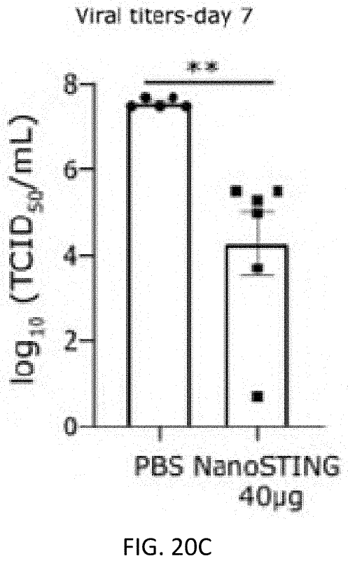

Next, an experiment was designed to test the impact of NanoSTING treatment on viral loads of lung tissue. Groups of ten mice were treated with a single intranasal dose of NanoSTING (40 μg) and 24 h later challenged with 4×LD50 (lethal dose 50) resistant strain of Influenza A (FIG. 20A). Animals were evaluated for 7 days with weight change and viral titers used as the primary endpoints. In this experiment, oseltamivir treatment had no significant effect, and 90% of mice succumbed to the disease. A single-dose treatment with NanoSTING protected animals from weight loss (mean peak weight loss at day 7 of 5.2% vs 32% placebo) (FIG. 20B). Following seven days after viral exposure, infectious particles in the lung were reduced by 500-fold compared to the placebo-treated group accounting for the ability of NanoSTING to help prevent disease and death (FIG. 20C).

Taken together, these results illustrated that NanoSTING treatment was effective against oseltamivir-resistant strains of Influenza A. These experiments confirmed that NanoSTING works as a broad spectrum antiviral against Influenza, including treatment-resistant strains.

### Example 9: Preparation of STING-Loaded Liposomes and Vaccine Formulation

2′-3″cyclic guanosine monophosphate adenosine monophosphate (cGAMP) was purchased from Chemietek (Indianapolis, Ind.). 1,2-dipalmitoyl-sn-glycero-3-phosphocholine (DPPC), 1,2-dipalmitoyl-sn-glycero-3-phospho-(1′-rac-glycerol) (DPPG), and 1,2-dipalmitoyl-sn-glycero-3-phosphoethanolamine-N-[methoxy(polyethylene-glycol)-2000] (DPPEPEG2000) were obtained from Avanti Polar Lipids (Alabaster, Ala.). We obtained the cholesterol from Sigma Aldrich (St. Louis, Mo.).

The liposomes were composed of a molar ratio of 10:1:1:1 of DPPC, DPPG, Cholesterol (Chol), and DPPE-PEG2000. To prepare the liposomes, we mixed the lipids in CHCl3 and CH₃OH, and we used a vacuum rotary evaporator for evaporating the solution for approximately 80 minutes at 45° C. We dried the resulting lipid thin film until all organic solvent evaporated. Next, we hydrated the lipid film by adding a pre-warmed cGAMP solution (0.3 mg/ml in PBS buffer, pH 7.4). We mixed the hydrated lipids at an elevated temperature 65° C. for an additional 30 minutes, then subjected them to freeze-thaw cycles. We then sonicated the mixture for 60 minutes using a Brandson Sonicator (40 kHz). Next, we removed the free un-trapped cGAMP by Amicon Ultrafiltration units (MW cut off 10 kDa). Subsequently, we washed the cGAMP-liposomes three times using PBS buffer. We measured the cGAMP concentration in the filtrates by Take3 Micro-Volume absorbance analyzer of Cytation 5 (BioTek) against a calibration curve of cGAMP at 260 nm. We calculated the final concentration of liposomal encapsulated cGAMP and encapsulation efficiency by subtracting the concentration of free drug in the filtrate. We purchased the SARS-CoV-2 (2019-nCoV) Nucleocapsid recombinant protein from BEI Resources (VA, USA #NR-53797) and tested it at two different doses (10 μg and 20 μg) in combination with the STING-liposomal suspensions. We kept the vaccine formulations at RT to allow the adsorption of the protein onto the liposomes. We stored the formulated vaccine at 4° C. and used it for up to 2 months. The average particle diameter, polydispersity index, and zeta potential were characterized by Litesizer 500 (Anton Paar) at RT.

THP-1 DUAL cell line (Invivogen) were cultured in a humidified incubator at 37° C. and 5% CO2 and grown in RPMI/10% FCS (Corning, N.Y., USA). In addition, we supplemented the THP-1 dual cell line with the respective selection agents (100 μg/ml zeocin+10 μg/ml blasticidin) and the corresponding selection cytostatics from Invivogen.

### Example 10: Preparation of STING-Loaded Liposomes and Vaccine Formulation

Cell Stimulation Experiments with Luciferase Reporter Enzyme Detection

We performed the cell stimulation experiments using the manufacturer's instructions (Invivogen, CA, USA). First, we seeded the cells in 96 well plate at 1×105 cells/well in 180 μl growth media. Next, we made serial dilutions of NanoSTING in growth medium. We then incubated the cells at 37° C. for 24 h. For detection of IRF activity, we collected 10 μl of culture supernatant/well at time points of 6 h, 12 h, and 24 h and transferred to a white (opaque) 96 well plate. Next, we read the plate on Cytation 7 (Cytation 7, Bio-Tek Instruments, Inc.) after adding 50 μl QUANTI-Luc™ (Invivogen) substrate solution per well followed by immediate luminescence measurement, which was given as relative light units (RLU).

### Example 11: Cytotoxicity Testing Formulation

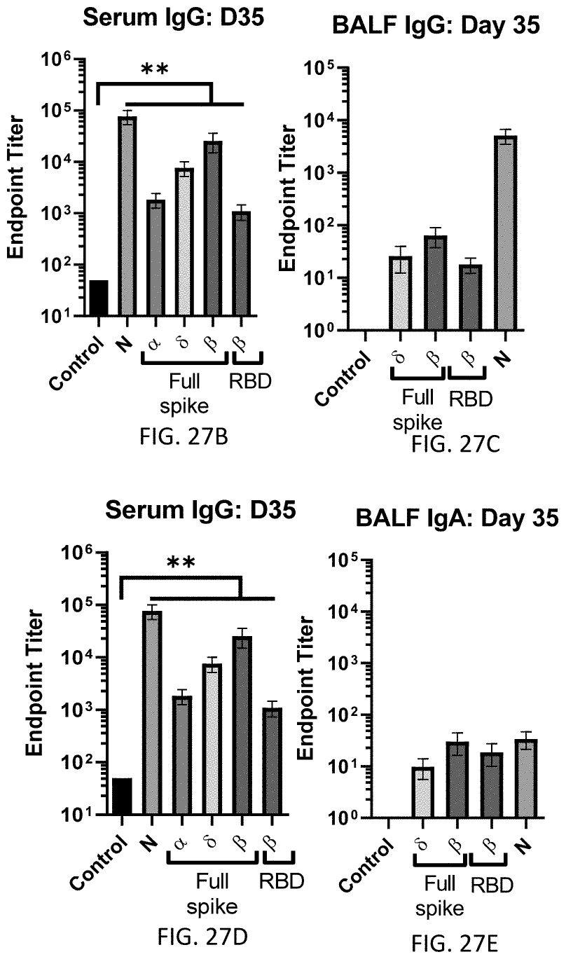

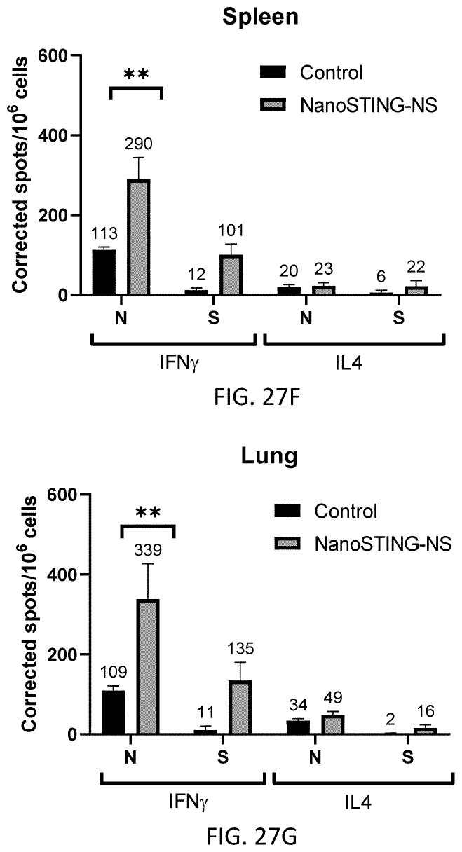

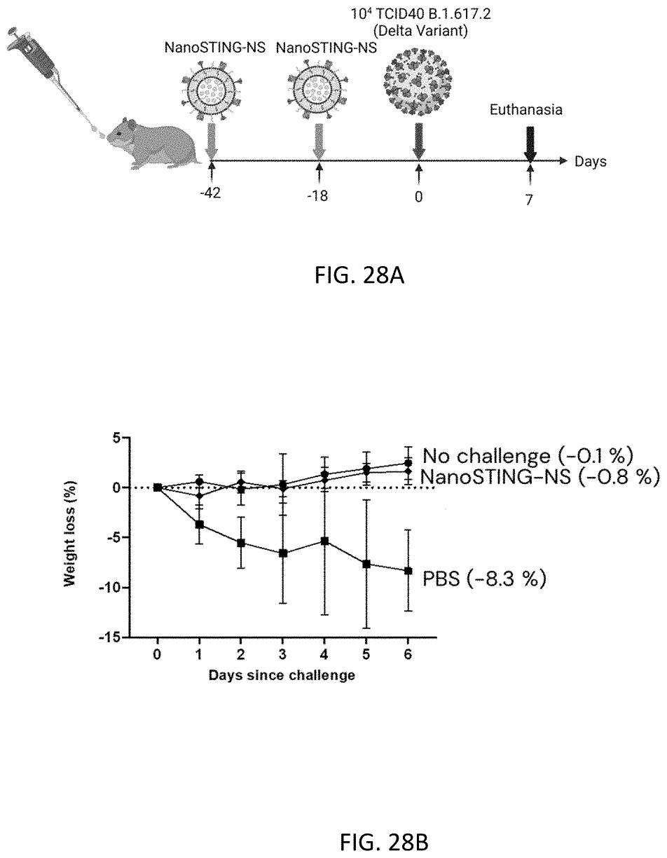

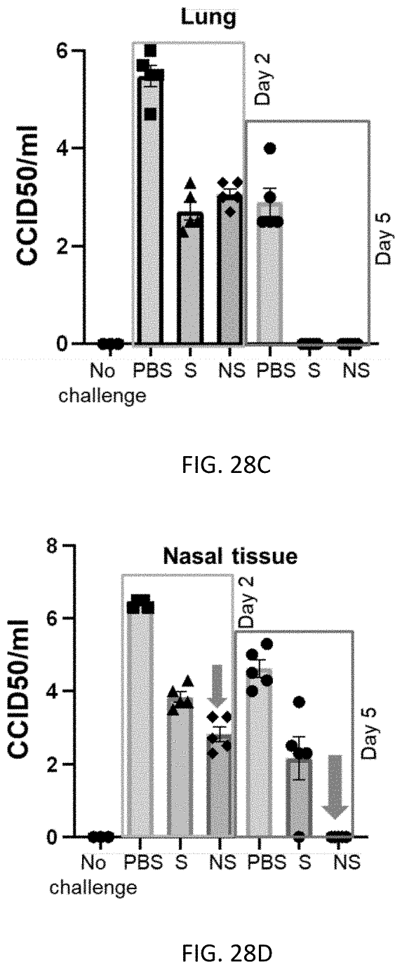

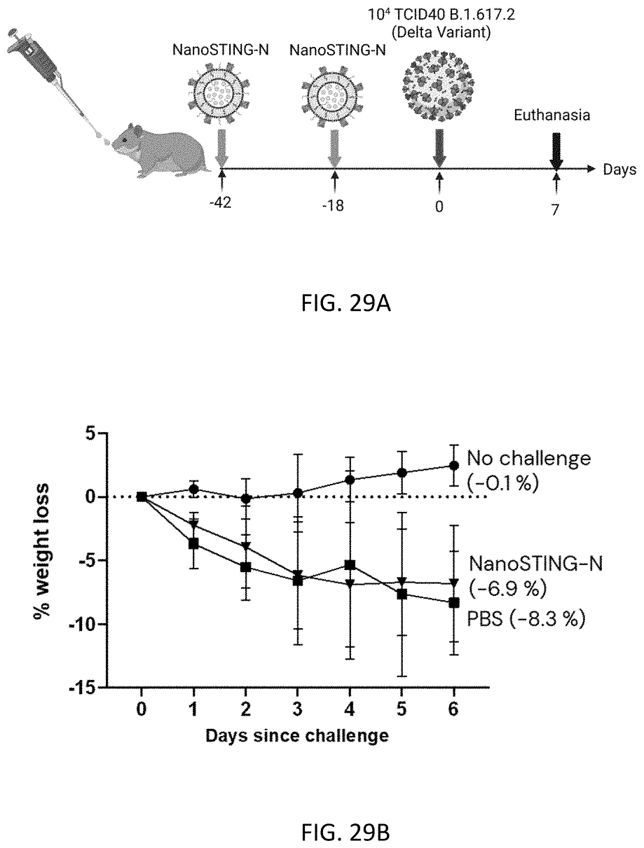

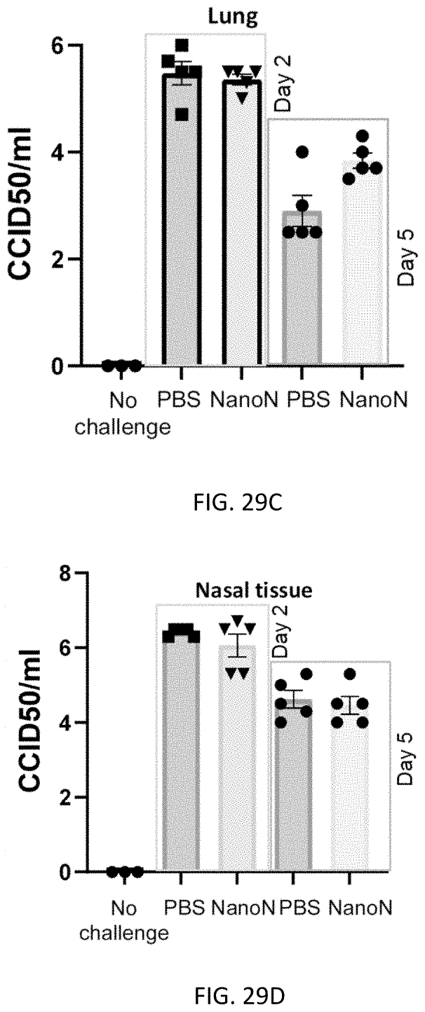

The cytotoxic activity of NanoSTING compositions was assessed by the MTT Assay. First, THP1-DUAL cells were incubated with NanoSTING at concentrations ranging from 46 μg/ml-5.75 μg/ml/24 hour period. After that, the cells were treated with 3-[4,5-dimethylthiazol-2-yl]-2,5-diphenyltetrazolium bromide (MTT) (Sigma Chemical Co., St. Louis, Mo.). Four hours later, all media was aspirated, including MTT solution (5 mg/ml), from the wells. The remaining formazan crystals were dissolved in DMSO, and the absorbance was measured at 570 nm using a 96 well microplate reader (Cytation 7, Bio-Tek Instruments, Inc.). The data in FIG. 27A and FIG. 27B were expressed as a percentage of viability compared with untreated cells (negative control—considered as 100% of viability) and cells treated with Triton X-100 (positive control—considered as 100% of cell death).

### Example 12: DNA Binding Assays

DNA binding studies were performed to check the applicability of the assay for detecting DNA condensation, we used branched-chain PEI as a positive control (Sigma Chemical Co., St. Louis, Mo. #408727). DiYO-1 (AAT Biorequest #17579) and Plasmid (pMB57.6)-DNA complexes were mixed in equal volumes of DNA and DiYO-1 (in 20 mM HEPES, 100 mM NaCl, pH=7.4) to achieve a final concentration of 400 nM and 8 nM, respectively. The solution was left at RT for 5 h before use. Next, PEI was added at different concentrations (R=0, 1, 2, 5 where R is the molar ratio of PEI Nitrogen to DNA phosphate) to DNA-DiYO-1 solution, vortexed for 1 minute and left for 2 h to equilibrate. Fluorescence intensity of the solution was measured at excitation and emission wavelength of 470 nm and 510 nm, respectively. The same procedure was also performed with SARS-CoV2 Nprotein instead of PEI. N protein was added to the DNA-DiYO-1 solution at concentrations of 0.1 and 0.5 μM.

### Example 13: Mice and Immunization

Female, 7-9-week-old BALB/c mice from Charles River Laboratories. Before administration of compounds, the mice were anesthetized by intraperitoneal injection of ketamine and xylazine. Animals were dosed with compositions having one of two different concentrations of the Nucleocapsid protein (10 μg and 20 μg) and 20 μg of the liposome-STING adjuvant.

### Example 14: Bodyweight Monitoring and Sample Collection

The bodyweight of animals was monitored every 7 days over four weeks after administration of NanoSTING composition or control treatment. Sera was collected at 7th, 14th, 21st, and 27th days post-administration for detection of the humoral immune response. Blood was maintained at room temperature (RT) for 10 minutes to facilitate clotting and then centrifuged for 5 minutes at 2000 g. The serum was collected and stored it at −80° C., and subsequently used for ELISA. Brochoalveolar lavage fluid (BALF), lung, and spleen were collected 27 days after the intranasal administration. Sera and other biological fluids (with protease inhibitors) were maintained at −80° C. for long-term storage. After dissociation, the splenocytes and lung lymphocytes were frozen in FBS+10% DMSO and stored in the liquid nitrogen vapor phase until further use.

### Example 15: ELISA Assays

Anti-N protein antibody titers were determined in serum or other biological fluids using ELISA. Briefly, 1 μg/ml N protein (Sino Biological, PA, USA) was coated onto ELISA plates (Corning, N.Y., USA) in PBS overnight at 4° C. for 2 hours at 37° C. The plate was then blocked with PBS+1% BSA (Fisher Scientific, PA, USA)+0.1% Tween20™ (Sigma-Aldrich, MD, USA) for 2 hours at RT. After washing, the samples were added at different dilutions. The captured antibodies were detected by HRP-conjugated anti-mouse IgG (Jackson ImmunoResearch Laboratories, 1 in 5,000; PA, USA), anti-mouse IgA (Bethyl Laboratories, 1:10,000; TX, USA), and detection antibody against mouse IgA (1:250) from the mouse total IgA ELISA kit from Invitrogen (CA, USA). The positive control (anti-N IgG) was obtained from Abeomics (CA, USA).

### Example 16: Processing of Spleen and Lungs for ELISPOT and Flow Cytometry

For isolation of lung lymphocytes, the lung vasculature was perfused with 5 ml of 1 mM EDTA in PBS w/o Ca2+Mg2+ injecting into the right cardiac ventricle. Each lung was cut into 100-300 mm² pieces using a scalpel. The minced tissue was transferred to a tube containing 5 ml of Digestion Buffer containing Collagenase D (2 mg/ml, Roche #11088858001) and DNase (0.125 mg/ml, Sigma #DN25) in 5 ml of RPMI for 1 h and 30 minutes at 37° C. in the water bath by vortexing after every 10 min. The remaining intact tissue was disrupted by passage (6-8 times) through a 21-gauge needle. Samples were incubated at room temperature for 90 min and 500 μl of ice cold stopping Buffer (1×PBS, 0.1M EDTA) was added to stop the reaction. Tissue fragments and dead cells were removed by a 40 μm disposable cell strainer (Falcon), and the cells were collected after centrifugation at 300 g. The red blood cells were lysed by resuspending the cell pellet in 3 ml of ACK Lysing Buffer (Invitrogen) and incubating for 3 minutes at RT, followed by centrifugation at 300 g. The supernatants were discarded, and the cell pellets were resuspended in 5 ml of complete RPMI medium (Corning, N.Y., USA). Next, the spleens in RPMI medium and homogenized through a 40 μm cell strainer using the hard end of a syringe plunger. After that, splenocytes were incubated in 3 ml of ACK lysis buffer for 3 minutes at RT to remove red blood cells (RBCs), then passed through a 40 μm strainer to obtain a single-cell suspension. Lung lymphocytes and splenocytes were counted by the trypan blue exclusion method.

### Example 17: ELISPOT Assays

IFN-γ and IL-4 ELISpot assays were performed using Mouse IFN-γ ELISPOT Basic kit (ALP) and Mouse IL-4 ELISPOT Basic Kit and following the manufacturer's instructions (Mabtech, VA, USA). For cell activation control, cultures were treated with 10 ng/ml phorbol 12-myristate 13-acetate PMA (Sigma, St. Louis, Mich., USA) and 1 μg/ml of ionomycin (Sigma, St. Louis, Mich., USA). The complete medium (RPMI supplemented with 10% FBS) was used as the negative control. Splenocytes and lung lymphocytes (3×10⁵) were stimulated in vitro with a pool of peptides, consisting mainly of 15-mer sequences with 11 amino acids overlap, covering the complete sequence of the nucleocapsid phosphoprotein (“N”) of SARS-Coronavirus 2 at a concentration of 1.5 μg/ml/peptide (Miltenyi Biotec; 130-126-699, Germany) at 37° C. for 16-18 h (hours) in precoated ELISpot plate (MSIPS4W10 from Millipore) coated with AN18 IFN-γ (1 μg/ml, Mabtech #3321-3-250;) and 11B11 IL-4 (1 μg/ml, Mabtech #3311-3-250) coating antibody. The next day, cells were washed and the plates were developed using biotinylated R4-6A2 anti-IFNγ (Mabtech #3321-6-250) and BVD6-24G2 anti-IL-4 (Mabtech #3311-6-250) detection antibody, respectively. The wells were washed and then treated for 1 h at RT with 1:30,000 diluted Extravidin-ALP Antibody (Sigma, St. Louis, Mich., USA). After washing, the spots were developed by adding 70 μL/well of BCIP/NBT-plus substrate (Mabtech #3650-10) to the wells. The plate was incubated for 20-30 minutes for color development and subsequently washed with water. The spots were quantified using Cytation 7 (Bio-Tek Instruments, Inc.). Each spot corresponds to an individual cytokine-secreting cell. Values were analyzed as the background-subtracted average of measured triplicates.

### Example 18: Cell Surface Staining, Intracellular Cytokine Staining for Flow Cytometry

The spleen and lung lymphocytes from immunized and control animals were stimulated for detecting Nucleocapsid protein-specific CD8+ T cell responses post 18 hours stimulation with Nucleocapsid protein-peptide pool at a concentration of 1.5 μg/ml/peptide (Miltenyi Biotec; 130-126-699, Germany) at 37° C. for 16-18 hours followed by the addition of Brefeldin A (5 μg/ml BD Biosciences #BD 555029) for the last 5 hours of the incubation. 10 ng/ml PMA (Sigma, St. Louis, Mich., USA) and 1 μg/ml ionomycin (Sigma, St. Louis, Mich., USA) was used as the positive control. Stimulation without peptides served as background control. Cells were collected and stained with Live/Dead Aqua (Thermo Fisher #L34965) in PBS, followed by Fc-receptor blockade with anti-CD16/CD32 (Thermo Fisher #14-0161-85), and then stained for 30 minutes on ice with the following antibodies in flow cytometry staining buffer (FACS): anti-CD4 AF589 (clone GK1.5; Biolegend #100446), anti-CD8b (clone YTS156.7.7; Biolegend #126609), anti-CD69 (clone H1.2F3; Biolegend #104537), anti-CD137 (clone 1AH2; BD; #740364), anti-CCR7 (clone 4B12; Biolegend #120124), anti-CD45 (clone 30-F11; BD; #564279). The cells were washed twice with the FACS buffer and then fixed them with 100 μl IC fixation buffer (eBioscience) for 30 minutes at RT. The cells were permeabilized for 10 minutes with 200 μl permeabilization buffer (BD Cytofix solution kit). Intracellular staining was performed using the antibodies Alexa Fluor 488 interferon (IFN) gamma (clone XMG1.2; BD; #557735) and Granzyme B (clone GB11; Biolegend; #515407) overnight at 4 C. Next, cells were washed with FACS buffer and analyzed them on LSR-Fortessa flow cytometer (BD Bioscience), using FlowJo™ software version 10.8 (Tree Star Inc, Ashland, Oreg., USA). Results were calculated as the total number of cytokine-positive cells with background subtracted. The amount of the antibodies used were optimized by titration.

### Example 19: Preparation and Characterization and Compositions

This example shows that NanoSTING is a liposomal adjuvant that enables mucosal immunity. The liposomal nanoparticles promote stability and have been shown to promote delivery to alveoloar macrophages, facilitating responses in the lung. Dynamic light scattering (DLS) analysis showed that the mean particle diameter of NanoSTING was 100 nm, with a polydispersity index of 0.21. The zeta potential of NanoSTING was −50 mV. The ability of NanoSTING to induce interferon (IRF) responses was confirmed by using the THP-1 monocytic cells that stably express secreted luciferase downstream of an IRF responsive promoter. THP-1 dual cells were stimulated with NanoSTING at concentrations ranging from 1.2-9.3 μg and performed kinetic measurements for 24 hours by measuring the luciferase activity in the supernatant. Although a low level of luciferase secretion was observed at 6 h, the secretion was maximal at 24 h and at this timepoint all of the concentrations tested showed similar responses. Taken together these results illustrated that optimal activation of the STING pathway by these NanoSTING compositions was observed at 24 h.

As the immunogen, the trimeric S protein based on the SARS-CoV-2 B.1.351 (beta) containing additional proline and alanine substitutions to confer stability was used. A single-step “mix and immunize” approach was utilized to enable the adsorption of the protein on to the liposomes. Using a standard S-protein quantitative ELISA, it was confirmed that 61.5% of the S-protein was adsorbed onto the liposomes. The adsorbed trimeric S-protein (NanoSTING-S) displayed a mean particle diameter of 104 nm and a mean zeta potential of 133 mV, with a polydispersity index of 0.24. In comparison to NanoSTING (81 nm, −35 mV), NanoSTING-S is more negatively charged as expected based on the isoelectric point (pI) of the protein. Unlike the trimeric S protein that is known to aggregate in solution, NanoSTING-S was tested after six months of storage at 4° C. and confirmed no evidence of aggregation or change in zeta potential.

### Example 20: Single-Dose Immunization of Mice with NanoSTING-S Vaccine Yields Cross-Reactive Humoral and Cellular Immunity

Mice were immunized with a single intranasal dose of NanoSTING-S and observed no clinical symptoms including loss of weight during the entire period of observation. 100% seroconversion was observed by day 7, and the response peaked by day 21. ELISA was conducted at day 28 to quantify binding to both variant full-length proteins and the receptor binding domain (RBD) proteins as a surrogate for neutralization. High serum IgG titers were observed not only against Beta but also against Alpha (B.1.1.7), Gamma (P.1) and Delta (B.1.617.2) full-length proteins. High serum IgG titers were also observed against the RBDs of both the Beta and Alpha variants. The SARS-CoV-2-specific antibody responses were assessed in bronchoalveolar lavage fluid (BALF) and confirmed robust IgG titers of against the full-length Beta. IgA-mediated protection is an essential component of mucosal immunity for respiratory pathogens. To confirm the role of intranasal NanoSTING as a mucosal adjuvant, IgA responses were tested in the serum. Strong IgA responses were detected against all three variants tested. Collectively these results established that a single-dose immunization with NanoSTING-S yielded robust IgG and IgA responses that is cross-reactive against the different spike variants including the Delta variant.

To assess the vaccine-induced S-specific T cell responses, splenocytes and lung lymphocytes were harvested 28 days after immunization. The T cells derived from spleen and lung were stimulated with a pool of overlapping 15-mer peptides and quantified antigen-specific T cells using IFN-γ (Th1/Tc1 responses) and IL-4 (Th2 responses) ELISPOT assays. NanoSTING-S immunized mice showed robust and significant lung and splenic T cell responses with a mean of 500 and 176 IFN-γ spots/10⁶ cells, respectively. The lung and spleen cells were also stimulated with a pool of peptides containing mutations in the S protein that are different from the Wuhan S protein. A significant Th1 response against these mutation-specific S peptides was observed confirming a broad T-cell response that targets both the conserved regions and the mutated regions of the S protein. In contrast to the IFN-γ (Th1/Tc1) responses, no measurable IL-4 (Th2) responses were observed upon immunization with NanoSTING-S. Collectively, these results established that intranasal vaccination elicited strong and cross-reactive Th1/Tc1 responses with no evidence of Th2 responses.

### Example 21: NanoSTING-S Elicited Immune Responses Confer Protection Against the Highly Infectious Delta Strain

To test the protective efficacy of NanoSTING-S, the golden hamster challenge model was used. This animal model replicates more severe disease in humans, and animals demonstrate easily quantifiable clinical disease characterized by rapid weight loss, very high viral loads in the lungs and extensive lung pathology. Additionally, unlike the K18-hACE2 model, hamsters recover from the disease (like humans) and hence offer the opportunity to study the impact of treatments both in the lungs (disease) and nasal passage (transmission). The animals were challenged with the Delta strain for two reasons: (1) the Delta strain is highly infectious, causes severe lung damage and has become a dominant strain in humans, and (2) Delta specific S-mutations including L452R and T478K within the RBD are absent in the immunogen, and, hence, challenge with Delta provided an opportunity to assess cross-protection.

Based on the findings with vaccines targeting other respiratory pathogens, two dose intranasal immunization of groups of ten hamsters with NanoSTING-S was used. The immunized hamsters were challenged with 10⁴ TCID50 of the Delta variant through the intranasal route. Animals in the sham-vaccinated group showed severe weight loss, with mean peak weight loss of 8.3%. By contrast animals vaccinated with NanoSTING-S were largely protected from weight loss (mean peak weight loss of 2.3%), similar to the results obtained by adenovirally vectored vaccines challenged with either the Wuhan or Beta strains. Half the animals were sacrificed at day 2 (peak of viral replication) and the other half at day 6 (peak of weight loss in unimmunized animals) to quantify viral loads. Even with the highly infectious Delta strain, NanoSTING-S reduced infectious viral loads in the lung by 300-fold by day 2 compared to sham-vaccinated animals; and by day 5, infectious virus was undetectable in all animals. Viral replication in the lung of the animals models clinical human disease and death, while viral replication in the nasal compartment models human transmission. Immunization with NanoSTING-S reduced infectious viral loads in the nasal compartment by 380-fold by day 2 compared to unimmunized animals. By day 5, vaccinated animals showed a further significant reduction in infectious virus. This reduction in viral loads in the nasal compartment is superior to intramuscularly delivered adenoviral vaccines and suggests an advantage of mucosal vaccination.

The lung tissue collected at day 6 after challenge was analyzed by using an integrated scoring rubric (range from 1-12) to account for both pathology and disease severity. Marked immune cell infiltration and widespread viral pneumonia in the lungs of sham-vaccinated hamsters were observed. It was observed that vaccinated and challenged animals had significant reductions in aggregate pathology score with minimal evidence of invasion by inflammatory cells or alveolar damage. In aggregate, these results from NanoSTING-S immunized hamsters challenged with the Delta strain coronavirus challenge demonstrates that NanoSTING compositions are cross-protective, are able to effectively protect the lung, and minimizes infectious virus in the nasal passage.

### Example 22: Single-Dose Immunization of Mice with NanoSTING-N Vaccine Yields Durable Humoral and Cellular Immunity

Since the N protein is largely conserved across the different SARS-CoV-2 variants, an N protein based vaccine was investigated. Independent studies with K18-hACE2 mice immunized with viral vector based N protein, and challenged the early lineage variants (Wuhan and Alpha) show mixed results with either partial protection or a complete lack of protection.

The SARS-CoV-2 N protein is predicted to be comprised of a nucleic acid (RNA) binding domain, a C-terminal dimerization domain and three intrinsically disordered domains that can promote phase separation with nucleic acids. Insect-cell derived recombinant N protein was used as the immunogen and SDS-PAGE confirmed a dominant band with a size of 47 kD. To confirm the functional capacity of the recombinant N protein, the interaction of N protein with plasmid DNA was studied. The DNA condensation probe DiYO-1, a bis-intercalating fluorophore whose quantum yield increases by several orders of magnitude upon binding to ds-DNA, was used. The N protein was able to quench the fluorescence of the DNA-DiYO-1 complex in a concentration-dependent manner. At a concentration of 0.5 μM the N protein decreased the fluorescence intensity to the same extent as the known synthetic polycation DNA condensation agent, polyethylenimine (PEI) R=5 (96.3%). These results confirmed that the recombinant N protein is potent to binder of dsDNA. To formulate the vaccine, NanoSTING-N, the recombinant N protein was gently mixed with NanoSTING to allow the adsorption of the protein on the liposomes. Although the N protein showed a strong propensity for aggregation upon storage at 4° C., NanoSTING-N was stable with no change in size or zeta potential. These results suggested that NanoSTING-N vaccine exists in a stable nanoparticulate colloidal form.

Two groups of mice were immunized by intranasal administration with liposomes comprising cGAMP and either 10 μg or 20 ug of Wuhan N protein (NanoSTING-N10 and NanoSTING-N20, respectively). Similar to the NanoSTING-S vaccine, NanoSTING-N10 and NanoSTING-N20 vaccinated animals reported no weight loss or gross abnormalities over 28 days. Fourteen days (d14) after immunization, 100% of the mice that received the NanoSTING-N seroconverted and robust anti-N IgG levels with mean dilution titers of 1:640 were detected and the responses increased to mean dilution titers of d27. By day 15 (d15), the serum concentration of the anti-S IgG antibodies increased, and mean dilution titers of 1:4,400 were detected. The IgG responses at both doses was similar at all of the timepoints tested and although the IgG titers elicited by the NanoSTING-N20 were higher than NanoSTING-N10, the difference was not significant. In contrast to vaccination with the trimeric NanoSTING-S (early response at day 7), the kinetics of IgG responses were delayed and responses were only observed at day 14. We assessed the SARS-CoV-2-specific antibody responses in the BALF; NanoSTING-N10 and NanoSTING-N20 showed mean IgG titers of 1:15 and 1:86 respectively. Consistent with the mucosal vaccination, serum IgA with NanoSTING-N10 (titer of 1:40) and Nano-STING-N20 (titers of 1:53) was observed. Collectively these results established that immunization with the N protein yielded robust humoral immunity in the serum and lung.

One of the concerns with N protein based vaccines is that Th2 responses towards the N protein can mediate antibody dependent enhancement (ADE) of viral infection. Accordingly, both Th1 and Th2 responses elicited in the lung and spleen of immunized animals were quantified.

Nano-STING-N10 and NanoSTING-N20 immunized mice showed robust and significant splenic T cell responses with a mean of 143 and 176 IFN-γ spots/106 cells, respectively. Animals immunized with NanoSTING-N10 and NanoSTING-N20 showed elevated T cell responses in the lung with a mean of 102 and 154 IFN-γ spots/106 respectively. In contrast to the IFN-γ (Th1/Tc1) responses, no measurable IL-4 (Th2) responses were observed upon immunization with NanoSTING-N10 and NanoSTING-N20. Collectively, these results established that intranasal vaccination elicited strong Th1 responses with no evidence of Th2 responses.

CD8+ T-cell responses can complement antibody mediated responses and can offer protection independent of antibody responses. The activation and function of N protein specific memory CD8+ T cells in the lung airways, lung parenchyma, and spleen were observed. The effector molecule granzyme B and the activation-induced marker CD137 to define N protein-reactive CD8+ T cells in the spleen and lungs were utilized. Re-stimulation ex vivo with a pool of N peptides resulted in a significant increase in the frequency of activated (CD8+CD137+) and cytotoxic (CD8+GzB+) T cells in the spleen, and to a lesser extent in the lung, of both the NanoSTING-N10 and NanoSTING-N20 vaccinated mice. The overall frequencies of the lung-resident CD103+CD69+CD8+ T cells was no different between the immunized animals and the control group. Taken together, these results established that NanoSTING-N elicits cytotoxic T-cell responses in the lung and spleen.

The immunogenicity of N protein based on the Alpha variant was also evaluated. Two key mutations R203K and G204R within the N protein have been fixed in all subsequent variants. Compositions were formulated based on lyophilized N protein, and mice immunized with NanoSTING-N showed robust serum IgG and IgA titers at day 28. The durability of the responses were also evaluated by measuring the serum IgG titers at day 62 and observed no reduction in either IgG or IgA. Similarly, it was confirmed that the N-reactive Th1 responses are conserved in the lung and spleen at day 62. Collectively, these mice studies confirmed that immunization with NanoSTING-N elicits strong cellular and humoral immune responses.

Based on the immunogenicity data in mice, the protective efficacy of NanoSTING-N in hamsters were evaluated. Hamsters were vaccinated with two doses of NanoSTING-N and challenged the immunized hamsters with 10⁴ TCID50 of the Delta variant through the intranasal route. Animals in both the vaccinated (mean peak weight loss of 6.9%) and sham-vaccinated group showed severe weight loss (mean peak weight loss of 8.3%). Consistent with the lack of protection from weight loss, infectious viral titers were no different in the lung or nasal passage at either day 2 or day 5 in both vaccinated and sham-vaccinated animals. In aggregate, these results from NanoSTING-N hamster experiments demonstrate that N protein as a single antigen is not sufficient to confer protection against the Delta strain.

### Example 23: Quantitative Modeling of the Combined Immune Response Against Both Proteins Predicts Synergistic Protection

The results from the NanoSTING-S experiments demonstrated that the immune responses protect against disease in the lung but are not sufficient to eliminate viral replication in the nasal passage as a surrogate for transmission. On the other hand, results from NanoSTING-N suggested that N protein while highly immunogenic, may not have a dominant role in protecting against SARS-CoV-2 infection in all cases. A mathematical model was used in combination with viral load data to quantify the opportunity for synergistic protection in the nasal compartment by vaccinating against both proteins.

The model was configured to track the viral load in the nasal passage by fitting the parameters to reflect longitudinal viral titers from infected patients. Vaccine induced neutralizing antibody responses against the spike protein serve as de novo blockers of viral entry and also block viral production through effector mechanisms. A range of vaccine efficacies (40 to 100%) were modeled to account for the differences in protection specifically in the nasal compartment and investigated the influence on viral elimination.

Cytotoxic T cell responses directed against the N protein directly kill virally infected cells thus directly reducing the number of cells capable of producing/propagating virus. For the NanoSTING-N vaccines that do not elicit neutralizing antibodies and are incapable of preventing viral entry, our model predicted that T cells would have efficacy. These estimates exceed the known efficacy of T-cell immunity and hence it is not surprising that single-antigen N based vaccines do not confer protection. Combined protection was modeled by including S directed vaccines that offer partial protection in the nasal compartment with the cytotoxic T cell responses against the N protein. When a range of S-protein vaccine efficacies (40 to 100%) was tested, in the nasal compartment, in combination with cytotoxic N responses, the model predicted that 1-3 cells needed to be eliminated with a day, an estimate well within the known range of T-cell killing frequencies. Indeed, studies in humans infected with COVID19 have demonstrated a robust and long-lived cytotoxic T cell response in the nasal compartment and that CD8+ T cells specific for the N protein can directly inhibit viral replication. When the viral reduction data from our hamster challenge models with NanoSTING-S was used, the model predicted that the N-directed cytotoxic response would only need to kill 1-3 cells/day within the nasal compartment. Collectively, these results from quantitative modeling predicted that combination vaccines targeting S and N proteins can mediate synergistic protection in eliminating viral replication in the nasal compartment.

### Example 24: Single-Dose Immunization of Mice with NanoSTING-NS Vaccine Yields Balanced Humoral and Cellular Immunity Targeting Both Proteins and Yields Sterilizing Immunity

To test the prediction from the quantitative model that the immune response against both the S and N proteins can be synergistic, vaccines containing both antigens were formulated. Immunogenicity experiments were initially performed in mice with 10 μg each of the N and S proteins adjuvanted with NanoSTING and it was observed that while 100% of animals seroconverted and showed IgG responses against the S protein, seroconversion against the N protein was variable (40-80%). The mass ratio of Alpha N: Beta S protein (2:1) was modified and adjuvanted with NanoSTING to formulate NanoSTING-NS. Single-dose intranasal vaccination with NanoSTING-NS yielded strong serum IgG titers against the Alpha N; full-length Alpha S, Beta S, and Delta S; and Beta S RBD. Strong antigen-specific, cross-reactive, IgG responses were documented in the BALF; and cross-reactive IgA responses were observed in the serum. Th1 responses were dominant against the N protein but were also significant against the S protein in both the lung and the spleen. Consistent with all other experiments, no evidence of Th2 response was observed in both tissues.

Based on these promising immunogenicity data in mice, the protective efficacy of NanoSTING-NS in hamsters was evaluated. Hamsters were vaccinated with two doses of NanoSTING-NS and the immunized hamsters were challenged with 10⁴ TCID50 of the Delta variant through the intranasal route. Animals immunized with NanoSTING-NS were completely protected from weight loss (mean peak weight loss of 0.8%). Exactly like the NanoSTING-S vaccine, NanoSTING-NS eliminated viral replication in the lung by day 6 post-challenge suggesting that 5-specific immune responses are the dominant factor in providing immunity in the lung. Pathology also confirmed that vaccinated and challenged animals had no minimal evidence of invasion by inflammatory cells or alveolar damage. In the nasal compartment, NanoSTING-NS showed a significant reduction in infectious viral particles by day 2 even in comparison to NanoSTING-S, and by day 6 there was a complete elimination of infectious viral particles in the NanoSTING-NS vaccinated animals. In aggregate, these results illustrate that sterilizing immunity can be accomplished by synergistic protection from immune responses against N in combination with immunity directed against the S protein.

Unless otherwise defined, all technical terms used herein have the same meaning as commonly understood by one of ordinary skill in the art to which this subject matter belongs. It is to be understood that both the foregoing general description and the following detailed description are illustrative and explanatory, and are not restrictive of the subject matter, as claimed.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As used herein, the singular forms “a,” “an,” and “the” are intended to include the plural forms as well, unless the context clearly indicates otherwise. For example, the word “a” or “an” means “at least one”, and the use of “or” means “and/or”, unless specifically stated otherwise. As used herein, the term “and/or” includes any and all combinations of one or more of the associated listed items. It will be further understood that the terms “comprises” and/or “comprising,” or “includes” and/or “including,” when used in this specification, specify the presence of stated features, regions, integers, steps, operations, elements and/or components, but do not preclude the presence of addition of one or more other features, regions, integers, steps, operations, elements, components, and/or groups thereof. Also, terms such as “element” or “component” encompass both elements or components comprising one unit and elements or components that comprise more than one unit unless specifically stated otherwise.

As used herein A and/or B encompasses one or more of A or B, and combinations thereof such as A and B. It will be understood that although the terms “first,” “second,” “third,” etc. may be used herein to describe various elements, components, regions and/or sections, these elements, components, regions and/or sections should not necessarily be limited by these terms. These terms may be used merely to distinguish one element, component, region or section from another element, component, region, or section. Thus, a first element, component, region or section discussed herein could be termed a second element, component, region or section without departing from the teachings of the present disclosure, in some cases.

The term “subject” as used herein includes mammals. Mammals include rats, mice, non-human primates, and primates, including humans.

The term “NanoSTING” is used herein to describe various embodiments of compositions described herein. For example, “NanoSTING” can refer to a composition comprising a modulator (e.g., a pattern recognition receptor agonist, such as a STING agonist) and a particle (e.g., a lipid-based particle, such as a lipid-based nanoparticle). In some cases, “NanoSTING” refers to a composition comprising a modulator and a particle, which does not have an associated antigen molecule. In some cases, the term NanoSTING refers to a composition comprising a modulator, a divalent cation (such as but not restricted to Mn2+, Mg2+, Ca2+, Zn2+) and a particle. In some cases, the term “NanoSTING” is used herein to refer to compositions described herein that include an antigen molecule, for example, when described in context of the inclusion of an antigen molecule and/or wherein the term is modified to reflect embodiments comprising an antigen molecule (e.g., “NanoSTING-Monomer,” “NanoSTING-Trimer,” “NanoSTING-RSV,” “NanoSTING-S,” “NanoSTING-varS,” “NanoSTING-ChimS,”, NanoSTING-N,” “NanoSTING-N10,” or “NanoSTING-N20”).

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

As used in this specification and the claims, unless otherwise stated, the term “about,” and “approximately,” or “substantially” refers to variations of less than or equal to +/−0.1%, +/−1%, +/−2%, +/−3%, +/−4%, +1-5%, +/−6%, +/−7%, +/−8%, +1-9%, +/−10%, +/−11%, +/−12%, +/−14%, +/−15%, or +/−20%, including increments therein, of the numerical value depending on the embodiment. As an example, in some cases, about 100 nanometers (nm) represents a range of 95 nanometers to 105 nanometers (which is +1-5% of 100 nanometers), 90 to 110 nanometers (which is +/−10% of 100 nanometers), or 85 nanometers to 115 nanometers (which is +/−15% of 100 nanometers) depending on the embodiments.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference for all purposes. All publications, patents, and patent applications mentioned in this specification are herein incorporated by reference to the same extent as if each individual publication, patent, or patent application was specifically and individually indicated to be incorporated by reference. The subject matter of Appendices A and B of U.S. Provisional Patent Application no. U.S. 63/301,918 and the subject matter of Appendix A and Appendix B of U.S. Provisional Patent Application no. U.S. 63/329,261, including all embodiments and combinations thereof described therein, are herein incorporated by reference for all purposes. In particular, embodiments and combinations thereof of compositions and/or methods comprising one or more particles, modulators, antigens, subjects, and/or experimental examples disclosed in U.S. Provisional Patent Application No. U.S. 63/301,918 (including Appendix A or Appendix B thereof) and/or in U.S. Provisional Patent Application No. U.S. 63/329,261 (including Appendix A or Appendix B thereof) are herein incorporated by reference for all purposes.

Although certain embodiments and examples are provided in the foregoing description, the inventive subject matter extends beyond the specifically disclosed embodiments to other alternative embodiments and/or uses, and to modifications and equivalents thereof. In any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not necessarily be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

1. A composition comprising:
a lipid-based particle and a modulator,
wherein the modulator is a pattern recognition receptor agonist, an activator of the immune system, or a combination thereof.

2. The composition of claim 1, wherein the modulator is encapsulated within the lipid-based particle.

3. The composition of claim 1, wherein the lipid-based particle comprises an antigen.

4. The composition of claim 1, wherein the lipid-based particle comprises a first antigen and a second antigen.

5. The composition of claim 4, wherein the first antigen is a spike protein molecule or portion thereof.

6. The composition of claim 5, wherein the second antigen is a nucleocapsid protein molecule or portion thereof.

7. The composition of claim 6, wherein the lipid-based particle comprises a greater quantity of nucleocapsid protein molecules than spike protein molecules.

8. The composition of claim 6, wherein the ratio of nucleocapsid protein molecules to spike protein molecules is at least 1:1.

9. The composition of claim 1, wherein the composition is formulated for intranasal delivery.

10. The composition of claim 1, wherein the modulator is an agonist of the STING pathway.

11. The composition of claim 1, wherein the lipid-based particle comprises DPPC, DPPG, cholesterol, and DPPE-PEG2000 in a 10:1:1:1 ratio.

12. The composition of claim 3, wherein the antigen is associated with an outer surface of the lipid-based particle.

13. The composition of claim 1, wherein the composition is lyophilized.

14. The composition of claim 1, wherein the composition is in liquid form.

15. The composition of claim 1, wherein the modulator is selected from the group consisting of bis-(3′,5′)-cyclic dimeric guanosine monophosphate (c-di-GMP), cyclic guanosine monophosphate-adenosine monophosphate (cGAMP), amidobenzimidazole, derivatives of amidobenzimidazole, nucleotide modulators, plasmid DNA modulators, divalent cations or combinations thereof.

16. The composition of claim 1, wherein the modulator comprises cyclic guanosine monophosphate-adenosine monophosphate (cGAMP).

17. A kit comprising a composition of claim 1, including instructions for use.

18. A method comprising:
administering a composition to a subject, wherein the composition comprises:
a lipid-based particle and a modulator,
wherein the modulator is a pattern recognition receptor agonist, an activator of the immune system, or a combination thereof.

19. The method of claim 18, wherein the subject does not exhibit symptoms of a respiratory disease.

20. The method of claim 18, wherein a sample obtained from the subject has a detectable level of a pathogen associated with the respiratory disease.

21. The method of claim 18, wherein a sample obtained from the subject does not have a detectable level of a pathogen associated with the respiratory disease.

22. The method of claim 18, wherein the subject exhibits symptoms of the respiratory disease.

23. The method of claim 18, wherein the composition is administered in at least one dose.

24. The method of claim 18, wherein the composition is administered in one dose.

25. The method of claim 18, wherein the composition is administered in two doses.

26. The method of claim 18, wherein the composition is administered to the subject before the subject is exposed to a pathogen associated with the respiratory disease.

27. The method of claim 26, wherein the composition is administered to the subject at least one day before the subject is exposed to a pathogen associated with the respiratory disease.

28. The method of claim 26, wherein the composition is administered to the subject at least three days before the subject is exposed to the pathogen associated with the respiratory disease.

29. The method of claim 18, wherein the composition is administered through intranasal administration.

30. The method of claim 18, wherein the composition is administered through inhalational administration

31. The method of claim 18, wherein the method is used to prevent the establishment of the disease in the subject, to prevent progression of the disease in the subject, to prevent the transmission of disease to a second subject, or combinations thereof.

32. The method of claim 18, wherein the method initiates an innate immune response that leads to associated adaptive immunity.

33. The method of claim 19, wherein the respiratory disease comprises an infection caused by a pathogen.

34. The method of claim 33, wherein the pathogen is a respiratory pathogen.

35. The method of claim 33, wherein the respiratory pathogen is a virus.

36. The method of claim 35, wherein the virus is selected from an influenza virus, a parainfluenza virus, an adenovirus, an enterovirus, a coronavirus, a respiratory syncytial virus, a rhinovirus, a DNA virus, an RNA virus, a variant thereof, or a combination thereof.

37. The method of claim 35, wherein the virus is an influenza virus.

38. The method of claim 37, wherein the influenza virus comprises an oseltamivir-sensitive strain, a treatment resistant strain, or a combination thereof.

39. The method of claim 35, wherein the virus is a coronavirus.

40. The method of claim 39, wherein the coronavirus is a SARS-CoV-2 virus, an alpha variant thereof, a delta variant thereof, an omicron variant thereof, or combinations thereof.


SEQUENCE LISTING (52 sequences)

SEQ ID NO: 1 - protein, 417 aa, synthetic construct
MSLNGPQNQMAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSMSSRNSTPGSSKRTSPARMAGNGGDAALALLLLDRLNQLESKMFGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - DNA, 1257 bp, synthetic construct
  source 1..1257: mol_type = other DNA
atgagcctgaacggcccgcagaaccagcgcaacgcgccgcgcattacctttggcggcccgagcgatagcaccggcagcaaccagaacggcgaacgcagcggcgcgcgcagcaaacagcgccgcccgcagggcctgccgaacaacaccgcgagctggtttaccgcgctgacccagcatggcaaagaagatctgaaatttccgcgcggccagggcgtgccgattaacaccaacagcagcccggatgatcagattggctattatcgccgcgcgacccgccgcattcgcggcggcgatggcaaaatgaaagatctgagcccgcgctggtatttttattatctgggcaccggcccggaagcgggcctgccgtatggcgcgaacaaagatggcattatttgggtggcgaccgaaggcgcgctgaacaccccgaaagatcatattggcacccgcaacccggcgaacaacgcggcgattgtgctgcagctgccgcagggcaccaccctgccgaaaggcttttatgcggaaggcagccgcggcggcagccaggcgagcagccgcagcagcagccgcagccgcaacagcagccgcaacagcaccccgggcagcagcaaacgcaccagcccggcgcgcatggcgggcaacggcggcgatgcggcgctggcgctgctgctgctggatcgcctgaaccagctggaaagcaaaatgtttggcaaaggccagcagcagcagggccagaccgtgaccaaaaaaagcgcggcggaagcgagcaaaaaaccgcgccagaaacgcaccgcgaccaaagcgtataacgtgacccaggcgtttggccgccgcggcccggaacagacccagggcaactttggcgatcaggaactgattcgccagggcaccgattataaacattggccgcagattgcgcagtttgcgccgagcgcgagcgcgttttttggcatgagccgcattggcatggaagtgaccccgagcggcacctggctgacctataccggcgcgattaaactggatgataaagatccgaactttaaagatcaggtgattctgctgaacaaacatattgatgcgtataaaacctttccgccgaccgaaccgaaaaaagataaaaaaaaaaaagcggatgaaacccaggcgctgccgcagcgccagaaaaaacagcagaccgtgaccctgctgccggcggcggatctggatgattttagcaaacagctgcagcagagcatgagcagcgcggatagcacccaggcg

SEQ ID NO: 3 - protein, 1194 aa, synthetic construct
VNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRAAASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQMFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 4 - DNA, 3585 bp, synthetic construct
  source 1..3585: mol_type = other DNA
gtgaactttaccacccgcacccagctgccgccggcgtataccaacagctttacccgcggcgtgtattatccggataaagtgtttcgcagcagcgtgctgcatagcacccaggatctgtttctgccgttttttagcaacgtgacctggtttcatgcgattcatgtgagcggcaccaacggcaccaaacgctttgcgaacccggtgctgccgtttaacgatggcgtgtattttgcgagcaccgaaaaaagcaacattattcgcggctggatttttggcaccaccctggatagcaaaacccagagcctgctgattgtgaacaacgcgaccaacgtggtgattaaagtgtgcgaatttcagttttgcaacgatccgtttctgggcgtgtattatcataaaaacaacaaaagctggatggaaagcgaatttcgcgtgtatagcagcgcgaacaactgcacctttgaatatgtgagccagccgtttctgatggatctggaaggcaaacagggcaactttaaaaacctgcgcgaatttgtgtttaaaaacattgatggctattttaaaatttatagcaaacataccccgattaacctggtgcgcggcctgccgcagggctttagcgcgctggaaccgctggtggatctgccgattggcattaacattacccgctttcagaccctgcatattagctatctgaccccgggcgatagcagcagcggctggaccgcgggcgcggcggcgtattatgtgggctatctgcagccgcgcacctttctgctgaaatataacgaaaacggcaccattaccgatgcggtggattgcgcgctggatccgctgagcgaaaccaaatgcaccctgaaaagctttaccgtggaaaaaggcatttatcagaccagcaactttcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaacattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtgaaaggctttaactgctattttccgctgcagagctatggctttcagccgacctatggcgtgggctatcagccgtatcgcgtggtggtgctgagctttgaactgctgcatgcgccggcgaccgtgtgcggcccgaaaaaaagcaccaacctggtgaaaaacaaatgcgtgaactttaactttaacggcctgaccggcaccggcgtgctgaccgaaagcaacaaaaaatttctgccgtttcagcagtttggccgcgatattgcggataccaccgatgcggtgcgcgatccgcagaccctggaaattctggatattaccccgtgcagctttggcggcgtgagcgtgattaccccgggcaccaacaccagcaaccaggtggcggtgctgtatcagggcgtgaactgcaccgaagtgccggtggcgattcatgcggatcagctgaccccgacctggcgcgtgtatagcaccggcagcaacgtgtttcagacccgcgcgggctgcctgattggcgcggaacatgtgaacaacagctatgaatgcgatattccgattggcgcgggcatttgcgcgagctatcagacccagaccaacagcccgcgcgcggcggcgagcgtggcgagccagagcattattgcgtataccatgagcctgggcgtggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagcccgattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcccggcgctgcagattccgtttccgatgcagatggcgtatcgctttaacggcattggcgtgacccagaacgtgctgtatgaaaaccagaaactgattgcgaaccagtttaacagcgcgattggcaaaattcaggatagcctgagcagcaccccgagcgcgctgggcaaactgcaggatgtggtgaaccagaacgcgcaggcgctgaacaccctggtgaaacagctgagcagcaactttggcgcgattagcagcgtgctgaacgatattctgagccgcctggatccgccggaagcggaagtgcagattgatcgcctgattaccggccgcctgcagagcctgcagacctatgtgacccagcagctgattcgcgcggcggaaattcgcgcgagcgcgaacctggcggcgaccaaaatgagcgaatgcgtgctgggccagagcaaacgcgtggatttttgcggcaaaggctatcatctgatgagctttccgcagagcgcgccgcatggcgtggtgtttctgcatgtgacctatgtgccggcgcaggaaaaaaactttaccaccgcgccggcgatttgccatgatggcaaagcgcattttccgcgcgaaggcgtgtttgtgagcaacggcacccattggtttgtgacccagcgcaacttttatgaaccgcagattattaccaccgataacacctttgtgagcggcaactgcgatgtggtgattggcattgtgaacaacaccgtgtatgatccgctgcagccggaactggatagctttaaagaagaactggataaatattttaaaaaccataccagcccggatgtggatctgggcgatattagcggcattaacgcgagcgtggtgaacattcagaaagaaattgatcgcctgaacgaagtggcgaaaaacctgaacgaaagcctgattgatctgcaggaactgggcaaatatgaacagtatattaaatggccg

SEQ ID NO: 5 - DNA, 20 bp, synthetic construct
  source 1..20: mol_type = other DNA
ctcccactcttccaccttcg

SEQ ID NO: 6 - DNA, 20 bp, synthetic construct
  source 1..20: mol_type = other DNA
gcctctcttgctcagtgtcc

SEQ ID NO: 7 - DNA, 20 bp, synthetic construct
  source 1..20: mol_type = other DNA
ctgatgctggtgacaaccac

SEQ ID NO: 8 - DNA, 20 bp, synthetic construct
  source 1..20: mol_type = other DNA
cagaattgccattgcacaac

SEQ ID NO: 9 - DNA, 20 bp, synthetic construct
  source 1..20: mol_type = other DNA
tgaattccctgggtgagaag

SEQ ID NO: 10 - DNA, 20 bp, synthetic construct
  source 1..20: mol_type = other DNA
ttcatggccttgtagacacc

SEQ ID NO: 11 - DNA, 20 bp, synthetic construct
  source 1..20: mol_type = other DNA
ctttgccatccaagagatgc

SEQ ID NO: 12 - DNA, 20 bp, synthetic construct
  source 1..20: mol_type = other DNA
tctcccacgtcaatctttcc

SEQ ID NO: 13 - DNA, 20 bp, synthetic construct
  source 1..20: mol_type = other DNA
tcgtgtttgtcactcgaagg

SEQ ID NO: 14 - DNA, 20 bp, synthetic construct
  source 1..20: mol_type = other DNA
agcaagcaatgacagggaag

SEQ ID NO: 15 - DNA, 20 bp, synthetic construct
  source 1..20: mol_type = other DNA
aagcagccagaagcagactc

SEQ ID NO: 16 - DNA, 20 bp, synthetic construct
  source 1..20: mol_type = other DNA
cagttctgacaccgtcatgg

SEQ ID NO: 17 - DNA, 20 bp, synthetic construct
  source 1..20: mol_type = other DNA
ccacgtgttgagatcattgc

SEQ ID NO: 18 - DNA, 20 bp, synthetic construct
  source 1..20: mol_type = other DNA
gaggctctctgctgtccatc

SEQ ID NO: 19 - DNA, 20 bp, synthetic construct
  source 1..20: mol_type = other DNA
tgtgcaggcactatgaggag

SEQ ID NO: 20 - DNA, 20 bp, synthetic construct
  source 1..20: mol_type = other DNA
actctggtccccaatgacag

SEQ ID NO: 21 - DNA, 20 bp, synthetic construct
  source 1..20: mol_type = other DNA
agagagactgaccgcagagc

SEQ ID NO: 22 - DNA, 20 bp, synthetic construct
  source 1..20: mol_type = other DNA
tcctcacttgcactctggtg

SEQ ID NO: 23 - DNA, 20 bp, synthetic construct
  source 1..20: mol_type = other DNA
acagcacagggcgttttatc

SEQ ID NO: 24 - DNA, 20 bp, synthetic construct
  source 1..20: mol_type = other DNA
gagcccagcattttctcttg

SEQ ID NO: 25 - DNA, 20 bp, synthetic construct
  source 1..20: mol_type = other DNA
tatggctcagggtccaactc

SEQ ID NO: 26 - DNA, 20 bp, synthetic construct
  source 1..20: mol_type = other DNA
ctccctttgcagaactcagg

SEQ ID NO: 27 - DNA, 20 bp, synthetic construct
  source 1..20: mol_type = other DNA
tgctgagatggactgtgagg

SEQ ID NO: 28 - DNA, 20 bp, synthetic construct
  source 1..20: mol_type = other DNA
tctggatttaaccggacagc

SEQ ID NO: 29 - DNA, 20 bp, synthetic construct
  source 1..20: mol_type = other DNA
gagaaggacctgaggtgcag

SEQ ID NO: 30 - DNA, 20 bp, synthetic construct
  source 1..20: mol_type = other DNA
ggagtgaatgtggctcagtg

SEQ ID NO: 31 - DNA, 20 bp, synthetic construct
  source 1..20: mol_type = other DNA
aggacatgaggtgcagttcc

SEQ ID NO: 32 - DNA, 20 bp, synthetic construct
  source 1..20: mol_type = other DNA
ggagtgaatgtggctcagtg

SEQ ID NO: 33 - DNA, 20 bp, synthetic construct
  source 1..20: mol_type = other DNA
ccaaggccaaccgtgaaaag

SEQ ID NO: 34 - DNA, 20 bp, synthetic construct
  source 1..20: mol_type = other DNA
atggctacgtacatggctgg

SEQ ID NO: 35 - DNA, 20 bp, synthetic construct
  source 1..20: mol_type = other DNA
tctatgaggtccggctgaca

SEQ ID NO: 36 - DNA, 20 bp, synthetic construct
  source 1..20: mol_type = other DNA
gcactggggctttaggtcat

SEQ ID NO: 37 - DNA, 20 bp, synthetic construct
  source 1..20: mol_type = other DNA
ccgcctcatacgggaaatgt

SEQ ID NO: 38 - DNA, 20 bp, synthetic construct
  source 1..20: mol_type = other DNA
aagacagaaggttgggctcg

SEQ ID NO: 39 - DNA, 20 bp, synthetic construct
  source 1..20: mol_type = other DNA
atttcggtcgcagggatctg

SEQ ID NO: 40 - DNA, 20 bp, synthetic construct
  source 1..20: mol_type = other DNA
tgcaagataaagcgtcccgt

SEQ ID NO: 41 - DNA, 20 bp, synthetic construct
  source 1..20: mol_type = other DNA
actgcctcgtgttcacatca

SEQ ID NO: 42 - DNA, 20 bp, synthetic construct
  source 1..20: mol_type = other DNA
ttcgggtgacaaaaacgact

SEQ ID NO: 43 - DNA, 23 bp, synthetic construct
  source 1..23: mol_type = other DNA
cctgaaagcacttgaagaattcc

SEQ ID NO: 44 - DNA, 23 bp, synthetic construct
  source 1..23: mol_type = other DNA
ggtatgctaaggcacagcacact

SEQ ID NO: 45 - DNA, 20 bp, synthetic construct
  source 1..20: mol_type = other DNA
gaaggaccagctggacaaca

SEQ ID NO: 46 - DNA, 21 bp, synthetic construct
  source 1..21: mol_type = other DNA
tggcaacccaagtaaccctta

SEQ ID NO: 47 - DNA, 20 bp, synthetic construct
  source 1..20: mol_type = other DNA
agctgcatttctgctgtggt

SEQ ID NO: 48 - DNA, 20 bp, synthetic construct
  source 1..20: mol_type = other DNA
ctgctttgtctggcctcaag

SEQ ID NO: 49 - DNA, 20 bp, synthetic construct
  source 1..20: mol_type = other DNA
acctgaccctgattgacctg

SEQ ID NO: 50 - DNA, 20 bp, synthetic construct
  source 1..20: mol_type = other DNA
accaggttgatggtctcctg

SEQ ID NO: 51 - DNA, 20 bp, synthetic construct
  source 1..20: mol_type = other DNA
aggagaccatcaacctggtg

SEQ ID NO: 52 - DNA, 20 bp, synthetic construct
  source 1..20: mol_type = other DNA
tcaggcttggtcaagattcc